(19) United States
(12) Patent Application Publication
(10) Pub. No.: US 2010/0278819 A1
(43) Pub. Date: Nov. 4, 2010

(54) STREPTOCOCCUS PNEUMONIAE VACCINES

(75) Inventors: Xavier Bossuyt, Leuven (BE); Leen Moens, Zoersel (BE)

Correspondence Address: DINSMORE & SHOHL LLP, FIFTH THIRD CENTER, ONE SOUTH MAIN STREET, SUITE 1300, DAYTON, OH, 45402-2023, US

(73) Assignee: KATHOLIEKE UNIVERSITEIT LEUVEN, Leuven (BE)

(21) Appl. No.: 12/677,829

(22) PCT Filed: Sep. 15, 2008
(86) PCT No.: PCT/EP08/07682

§ 371 Date: Apr. 14, 2010

Related U.S. Application Data
(60) Provisional application No. 60/974,394, filed on Sep. 21, 2007.

(30) Foreign Application Priority Data
Sep. 14, 2007 (GB) 0718222.3
Sep. 14, 2007 (GB) 0718225.6
Sep. 14, 2007 (GB) 0718226.4
Nov. 29, 2007 (GB) 0723335.6
Nov. 29, 2007 (GB) 0723336.4

(51) Int. Cl. A61K 39/40; C07K 14/315; A61K 39/09; C07K 16/12; C07H 21/04; A61P 31/04; A61P 37/04

(52) U.S. Cl. 424/133.1; 530/350; 424/244.1; 530/389.5; 424/165.1; 530/388.4; 536/23.7

(57) ABSTRACT

Streptococcus pneumoniae is a major cause of pneumoniae, meningitis, and major cause of morbidity and mortality throughout the world by bacterial otitis media, pneumoniae, meningitis, and bacteraemia. It is an important agent of disease in man especially among infants, the elderly and immunocompromised persons. The present invention provides a solution to this problem by providing a substantially pure or isolated disease related antigen selected from the group consisting of the isolated, recombinant or synthetic S. pneumoniae human immunogenic antigens of SP_0562, SP_0965, SP_0082 (in particular the fragments SP4 and SP 17 of said SP_0082), and a Periplasmic Binding Protein (PBP) (in particular SP_1683 or SP_1386), or a fragments thereof or substantially identical antigen for use in a treatment to induce a immunological memory in a human against S. pneumoniae cells for use in a vaccination treatment of S. pneumoniae disorder in human or against S. pneumoniae in a human. In a particular embodiment, the present invention provides an isolated, recombinant or synthetic S. pneumoniae Periplasmic Binding Protein (PBP) (in particular SP_1683 or SP_1386) as a disease related antigen for use in a treatment to induce a immunological memory in a human against S. pneumoniae cells for use in a vaccination treatment of S. pneumoniae disorder in human or for use in the treatment of an S. pnewnoniae infection in a human. It further provides antibodies that specifically bind to the S. pneumoniae disease related antigens identified herein for use in the treatment of a an S. pneumoniae infection in a human, such as for example in a treatment to induce immunological memory in a human against S. pneumoniae, i.e. in a vaccination treatment of S. pneumoniae. It is also an aspect of the present invention to provide the use of any one of the S. pneumoniae disease related antigens as identified herein, or of the antibodies specific for said antigens in methods to diagnose for a S. pneumoniae disorder in a human.

### BACKGROUND OF THE INVENTION

A. Field of the Invention

The present invention relates generally to protein based pneumococcal vaccines, to nucleic acids encoding such proteins and to the passive vaccines with antibodies against such proteins. Furthermore it relates to peptide, polypeptide or protein based pneumococcal vaccines and to isolated peptide, polypeptide or protein antigens that are immunogenic in human as components for use in prophylaxis, diagnostic and/or therapy of Streptococcus pneumoniae (S. pneumoniae) infection, or to induce a immunological memory in human subjects against such antigens. The invention relates furthermore to a vaccine against S. pneumoniae that comprises such isolated peptide, polypeptide or protein antigenic factors, which are immunogenic into humans, and to the use of such vaccine in a method of treatment of S. pneumoniae induced disorders and such as pneumoniae meningitis and bacteraemia. In a particular embodiment the invention relates human or humanized antibodies or against such isolated peptide, polypeptide or protein antigenic factors, which are immunogenic into humans, for use in a passive vaccine against S. pneumoniae and S. pneumoniae induced disorders in humans such as pneumoniae meningitis and bacteraemia and in particular to protect children or weak, elderly, critically ill or immunocompromised patients. Several documents are cited throughout the text of this specification. Each of the documents herein (including any manufacturer's specifications, instructions etc.) are hereby incorporated by reference; however, there is no admission that any document cited is indeed prior art of the present invention. The polynucleotides encoding these human immunogenic polypeptides. A further understanding of the nature and advantages of the present invention may be realized by reference to the remaining portions of the specification and claims. All publications, figures, GenBank Accession references (sequences), ATCC Deposits, patents and patent applications cited herein are hereby expressly incorporated by reference for all purposes to the same extent as if each was so individually denoted.

B. Description of the Related Art

S. pneumoniae is a major cause of pneumoniae, meningitis, and major cause of morbidity and mortality throughout the world by bacterial otitis media, pneumoniae, meningitis, and bacteraemia. It is an important agent of disease in man especially among infants, the elderly and immunocompromised persons. It is a bacterium frequently isolated from patients with invasive diseases such as bacteraemia/septicaemia, pneumoniae, meningitis with high morbidity and mortality throughout the world. Even with appropriate antibiotic therapy, pneumococcal infections still result in many deaths. Although the advent of antimicrobial drugs has reduced the overall mortality from pneumococcal disease, the presence of resistant pneumococcal organisms has become a major problem in the world today. Effective pneumococcal vaccines can have a major impact on the morbidity and mortality associated with S. pneumoniae disease. Such vaccines will particularly be useful to prevent otitis media in infants and young children. Passive vaccines based on human antibodies against S. pneumoniae proteins can be particularly suitable to protect immunocompromised patients or when a fast treatment is required. This is for instance the case instance critically ill patients who are affected by S. pneumoniae. S. pneumoniae remains the most common cause of death among critically ill patients in the

Intensive Care Units (ICU) of hospitals, who attained severe pneumoniae (Larry M. Baddour, et al. (2004) American Journal of Respiratory and Critical Care Medicine Vol 170. pp. 440-444). Ventilator-associated pneumoniae (VAP), a pneumoniae that arises more than 48-72 h after endotracheal intubation and is an important cause of morbidity and mortality of critically ill patients admitted to the intensive care unit (ICU). VAP occurs in 9-27% of all intubated patients (Chaste J and Fagon J Y. (2002) Am J Respir Crit Care Med 165:867-903 and Rello J, Ollendorf D A, Oster G, et al. (2002) Chest 122:2115-21). Early diagnosis for S. pneumoniae for correct timing of eventually treatment against S. pneumoniae of these patients is crucial. S. pneumoniae has been found to be responsible for early onset VAP (defined as occurring within the first 4 days of hospitalization. There is thus a clear need in the art for an efficient diagnosis and therapy in the Intensive Care Units.

Efforts to develop a pneumococcal vaccine have generally concentrated on generating immune responses to the pneumococcal capsular polysaccharide. Vaccination with pneumococcal polysaccharides is an effective means to prevent pneumococcal infections. The currently available pneumococcal vaccines have significant shortcomings related primarily to the poor immunogenicity of capsular polysaccharides, the inability of capsular polysaccharides to generate immunological memory, the fact that the capacity to produce antibodies against capsular polysaccharides age dependent is, the diversity of the serotypes and the differences in the distribution of serotypes in geographic areas. The use of an antigenically conserved immunogenic pneumococcal protein antigen, either by itself or in combination with additional components, offers the possibility of a protein-based pneumococcal vaccine.

PCT WO 98/18930 published May 7, 1998 entitled “Streptococcus Pneumoniae antigens and vaccines” describes certain polypeptides which are claimed to be antigenic and PCT WO 00/39299 describes polypeptides and polynucleotides encoding these polypeptides. PCT WO 00/39299 demonstrates that polypeptides designated as BVH-3 and BVH-11 provide protection against fatal experimental infection with pneumococci. However, the protein-based pneumococcal vaccine formulation of the current art needs further optimization

Thus, there is a need in the art for pneumococcal antigens with clear immunogenicty in human that may be used as components for the prophylaxis, diagnostic and/or therapy of pneumococcal infection and in particular for a (poly)peptide or protein-based vaccine as an alternative to a polysaccharide based vaccine.

Present invention demonstrated that the S. pneumoniae proteins of the group consisting of zmpB, GroEL, and ABC transporter (spermidine) and that proteins of the group of zmpC, ABC transporters (glutamine, maltose/maltodextrin, SP_{—}1683), PTS system IIA component (mannose), pyruvate kinase and proteins SP_{—}1290 and SP_{—}0562 are S. pneumoniae components that are immunogenic in a human immune system. Moreover the present invention demonstrated that such isolated polypeptide or protein components that are immunogenic in human or combinations there of can be used in prophylaxis, diagnostic and/or therapy of Streptococcus, in particular S. pneumoniae infection

### SUMMARY OF THE INVENTION

The present invention solves the problems of the related art of S. pneumoniae infections a major cause of morbidity and mortality, the presence of pneumococcal organisms that are resistant to antibiotics and the shortcomings of current pneumococcal vaccines related primarily to the poor immunogenicity of capsular polysaccharides, the inability of capsular polysaccharides to generate immunological memory; the fact that the capacity to produce antibodies against capsular polysaccharides age dependent is, the diversity of the serotypes, the differences in the distribution of serotypes in geographic areas and the inability to create immunological memory by the use of an antigenically conserved and human immunogenic pneumococcal protein antigens, either by itself or in combination with additional components.

In accordance with the purpose of the invention, as embodied and broadly described herein, the invention is broadly drawn to protein based vaccines or the use of their encoding nucleic acids or antibodies against such protein antigens.

In one aspect of the invention, a substantially pure or isolated disease related antigen selected from the group consisting of the isolated, recombinant or synthetic S. pneumoniae human immunogenic antigens of Zinc metalloprotease C (zmpC), ABC transporters (glutamine, maltose/maltodextrin, SP_{—}1683), PTS system IIA component (mannose), pyruvate kinase and antigens SP_{—}1290 and SP_{—}0562, or a fragment thereof or substantially identical antigen is used in a treatment to induce a immunological memory in a human against S. pneumoniae for use in a vaccination treatment of S. pneumoniae disorder in human or against S. pneumoniae in a human. In a further embodiment the invention provides a substantially pure or isolated antigen selected from the group consisting of SP_{—}0562, SP_{—}0965, SP_{—}0082 (in particular the fragments SP4 and SP17 of said SP_{—}0082), and a Periplasmic Binding Protein (PBP) (in particular SP_{—}1683 or SP_{—}1386), or a fragments thereof or prevention of an S. pneumoniae infection in a subject in need thereof, such as for example in a treatment to induce a immunological memory in a human against S. pneumoniae, i.e. in a vaccination treatment of S. pneumoniae. In an even further embodiment the invention provides a substantially pure or isolated antigen selected from the group consisting of SP_{—}0562, SP_{—}1683 and SP_{—}1386 or a fragments thereof for use in or prevention of an S. pneumoniae infection in a subject in need thereof, such as for example in a treatment to induce a immunological memory in a human against S. pneumoniae, i.e. in a vaccination treatment of S. pneumoniae.

This substantially pure or isolated disease related antigen can be characterized in that consists of any one of the aforementioned isolated, recombinant or synthetic S. pneumoniae human immunogenic antigens in isolation, for use in a treatment to induce a immunological memory in a human against S. pneumoniae for use in a vaccination treatment of S. pneumoniae disorder in human or against S. pneumoniae in a human. It accordingly provides a substantially pure or isolated antigen wherein said antigen is Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment of any one of these antigens, for use in a treatment to induce a immunological memory in a human against S. pneumoniae for use in a vaccination treatment of S. pneumoniae disorder in human or against S. pneumoniae in a human. The substantially pure or isolated disease related antigen can be also characterized in that it is the human immunogenic antigen, SP_{—}0562, or a fragment thereof or substantially identical antigen for use in a treatment to induce a immunological memory in a human against S. pneumoniae for use in a vaccination treatment of S. pneumoniae disorder in human or against S. pneumoniae in a human. Present invention also concerns a the substantially pure or isolated disease related antigen that is characterized in that it is the human immunogenic antigen, SP_{—}0965 or a fragment thereof or substantially identical antigen for use in a treatment to induce a immunological memory in a human against S. pneumoniae for use in a vaccination treatment of S. pneumoniae disorder in human or against S. pneumoniae in a human. Furthermore substantially pure or isolated disease related antigen of present invention can be characterized in that it is a Periplasmic Binding Protein (PBP) (in particular SP_{—}1863 or SP_{—}1386) or a fragment thereof or a substantially identical antigen for use in a treatment to induce a immunological memory in a human against S. pneumoniae for use in a vaccination treatment of S. pneumoniae disorder in human or against S. pneumoniae in a human or it can be characterized in that it is the human immunogenic antigen, SP_{—}1683, or a fragment thereof or a substantially identical antigen for use in a treatment to induce a immunological memory in a human against S. pneumoniae for use in a vaccination treatment of S. pneumoniae disorder in human or against S. pneumoniae in a human. The substantially pure or isolated disease related antigen can be also characterized in that it is the human immunogenic antigen, SP_{—}1386, or a fragment thereof or substantially identical antigen for use in a treatment to induce a immunological memory in a human against S. pneumoniae for use in a vaccination treatment of S. pneumoniae disorder in human or against S. pneumoniae in a human.

Furthermore, the aforementioned substantially pure or isolated S. pneumoniae immunogenic antigens of the present invention, can be combined in the treatment of S. pneumoniae disorders in a human. It is thus an objective of the present invention to provide the use of two or more, in particular 3, 4, 5, 6, or 7 of the disease related antigen selected from the group consisting of the isolated, recombinant or synthetic S. pneumoniae human immunogenic antigens of SP_{—}0562, SP_{—}0965, SP_{—}0082 (in particular the fragments SP4 and SP17 of said SP 0082), SP 1683 and SP_{—}1386, or a fragments thereof, in the treatment or prevention of a S. pneumoniae infection in a subject in need thereof.

These substantially pure or isolated disease related antigens of the present invention, taken in isolation or in combination (supra), can be (further) combined with at least one human immunogenic antigen of the group consisting of Zinc metalloprotease B (zmpB), GroEL, and ABC transporter for spermidine/putrescie (potD) or a fragment thereof or a substantially identical antigen for use in a treatment to induce a immunological memory in a human against S. pneumoniae for use in a vaccination treatment of S. pneumoniae disorder in human or against S. pneumoniae in a human.

Furthermore the substantially pure or isolated disease related antigens of the present invention, taken in isolation or in combination (supra), can be (further) combined with at least one human immunogenic antigen of the group consisting of Pneumococcal histidine antigen A (PhpA, BHV-11), Pneumococcal surface antigen. A,. Pneumococcal surface antigen C, Pneumococcal surface adhesin A, ORF SP_{—}0082, Fructose biphosphate aldolase, Endo-β-N-acetylglucosaiminidase, IgA1 protease, Serine protease PrtA, α-enolase, Glyceraldehyde-3-phosphate dehydrogenase, DnaK, Pyruvate oxidase, ClpP protease and Phosphoglycerate kinase, or a fragment thereof or a substantially identical antigen for use in a treatment to induce a immunological memory in a human against S. pneumoniae for use in a vaccination treatment of S. pneumoniae disorder in human or against S. pneumoniae in a human. In one embodiment the group consists of any combination of the foregoing S. pneumoniae immunogenic antigens comprising at least one, two or three of the S. pneumoniae immunogenic antigens selected from SP_{—}0562, SP_{—}1683 and SP_{—}1386.

The substantially pure or isolated disease related antigen of present invention for use in a treatment to induce an immunological memory in a human against S. pneumoniae or for use in a vaccination treatment of S. pneumoniae disorder in human or against S. pneumoniae bacteria in a human can be characterised in that the antigen is a peptide, protein or polypeptide or it can be characterised in that the antigen is a nucleotide, for instance a nucleotide that operable linked to a vector. These substantially pure or isolated disease related antigens of present invention can be combined with an immunostimulant for use as vaccination agent in a vaccination treatment of S. pneumoniae disorder in human or against S. pneumoniae in a human.

In another aspect of the invention a substantially pure or isolated antibody that specifically binds to a disease related antigen, i.e. to one disease related antigen selected from the group consisting of the isolated, recombinant or synthetic S. pneumoniae human immunogenic antigens of Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof or a substantially identical antigen is used in a vaccination treatment of S. pneumoniae disorder in human or against S. pneumoniae in a human. In a further objective the antibodies specifically bind to a substantially pure or isolated antigen selected from the group consisting of SP_{—}0562, SP_{—}0965, SP_{—}0082 (in particular the fragments SP4 and SP17 of said SP_{—}0082), and a Periplasmic Binding Protein (PBP) (in particular SP_{—}1683 or SP_{—}1386), or a fragment thereof, for use in a vaccination treatment of S. pneumoniae disorder in a human or against S. pneumoniae in a human. Again, in a particumar embodiment the antibodies specifically bind to a substantially pure or isolated antigen selected from the group consisting of SP_{—}0562, SP_{—}1683 and SP_{—}1386.

As for the antigens hereinbefore the antigen specific antibodies of the present invention can be taken in isolation for use in the treatment of a S. pneumoniae infection in a subject in need thereof. As such, the invention provides an antibody that specifically bind to a substantially pure or isolated antigen, wherein said antigen consists of SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment of any of said antigens, for use in a vaccination treatment of S. pneumoniae disorder in a human or against S. pneumoniae in a human. In a particular embodiment the antibody selectively binds to a Periplasmic Binding Protein (PBP) (in particular SP_{—}1683 or SP_{—}1386), or a fragment thereof, for use in the treatment of a S. pneumoniae infection in a subject in need thereof, i.e. for use in a vaccination treatment of S. pneumoniae disorder in a human or against S. pneumoniae in a human. More in particular the antibody for use in a vaccination treatment of S. pneumoniae disorder in a human or against S. pneumoniae in a human, is specific for SP_{—}1683 or SP_{—}1386. Even more in particular the antibody for use in a vaccination treatment of S. pneumoniae disorder in a human or against S. pneumoniae in a human, is specific for SP_{—}1683. Alternatively, the antibody for use in a vaccination treatment of S. pneumoniae disorder in a human or against S. pneumoniae in a human, is specific for SP_{—}1386.

Such substantially pure or isolated antibodies of present invention can be combined with a substantially pure or isolated antibody that specifically binds to at least one human immunogenic antigen of the group consisting of Zinc metalloprotease B (zmpB), GroEL, and ABC transporter for spermidine/putrescie (potD) or a fragment thereof or a substantially identical antigen for use in a vaccination treatment of S. pneumoniae disorder in human or against S. pneumoniae in a human. Furthermore, the aforementioned antibodies that specifically bind the S. pneumoniae human immunogenic antigens of the present invention, can be combined in the treatment of S. pneumoniae disorders in a human. It is thus an objective of the present invention to provide the use of substantially pure or isolated antibodies that specifically binds to two or more, in particular 3, 4, 5, 6, or 7 of the disease related antigen selected from the group consisting of SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof, in the treatment of a S. pneumoniae infection in a subject in need thereof. Furthermore the substantially pure or isolated antibodies of the present invention, taken in isolation or in combination (supra), can be (further) combined with at least one substantially pure or isolated antibody that specifically binds to an antigen of the group consisting of human immunogenic antigen of the group consisting of Pneumococcal histidine antigen A (PhpA, BHV-11), Pneumococcal surface antigen A, Pneumococcal surface antigen C, Pneumococcal surface adhesin A, ORF SP_{—}0082, Fructose biphosphate aldolase, Endo-β-N-acetylglucosaminidase, IgA1 protease, Serine protease PrtA, α-enolase, Glyceraldehyde-3-phosphate dehydrogenase, DnaK, Pyruvate oxidase, ClpP protease and, Phosphoglycerate kinase, or a fragment thereof or a substantially identical antigen for use in a vaccination treatment of S. pneumoniae disorder in human or against S. pneumoniae in a human. As for the antigens for use in the treatment of an S. pneumoniae infection in a subject in need thereof, in a particular embodiment the group of antibodies as defined hereinbefore, comprise at least antibodies specific for at least one, two or three of the substantially pure or isolated antigen selected from the group consisting of SP_{—}0562, SP_{—}1683 and SP_{—}1386.

Such antibody can be a (recombinant) human antibody. It can be a diclonal antibody, an oligoclonal antibody, a polycloncal antibody, a rearranged antibody or a heterohybrid antibody.

In still another aspect of the invention, it provides the use of the substantially pure or isolated disease related antigen of the present invention or of the antibodies that selectively bind to said antigens, for use in a diagnostic treatment to diagnose for a S. pneumoniae disorder in a human. It thus provides in one instance, the substantially pure or isolated disease related antigen selected from the group consisting of the isolated, recombinant or synthetic S. pneumoniae human immunogenic antigens of Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof or substantially identical antigen for use in a diagnostic treatment to diagnose for a S. pneumoniae disorder in a human. In a further instance it provides the substantially pure or isolated disease related antigen selected from the group consisting of the isolated, recombinant or synthetic S. pneumoniae human immunogenic antigens of Zinc metalloprotease C (zmpC),

ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof or substantially identical antigen for use in a diagnostic treatment to diagnose for a S. pneumoniae disorder in a human.

In one embodiment the substantially pure or isolated antigens for use in a method to diagnose for a S. pneumoniae disorder in a human, is selected from the group consisting of SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof. Even more in particular selected from the group consisting of SP_{—}0562, SP_{—}1683 and SP_{—}1386. In said diagnostic methods the aforementioned S. pneumoniae disease related antigens can be used in isolation or; two or more, in particular 3, 4, 5, 6, or 7 of the disease related antigen selected from the group consisting of the isolated, recombinant or synthetic S. pneumoniae human immunogenic antigens of Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragments thereof, are used in a method to diagnose for a Streptococcus pneumoniae disorder in a human. In a particular embodiment two or more, in particular 3, 4, 5, 6, or 7 of the disease related antigen selected from the group consisting of SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof are used in a method to diagnose for a Streptococcus pneumoniae disorder in a human. In a particular embodiment the substantially pure or isolated antigens for use in a method to diagnose for a S. pneumoniae disorder in a human, is a Periplasmic Binding Protein (PBP) (in particular SP 1683 or SP_{—}1386), or a fragment thereof. More in particular the substantially pure or isolated antigens for use in a method to diagnose for a S. pneumoniae disorder in a human, is SP_{—}1683 or SP_{—}1386. Even more in particular the substantially pure or isolated antigens for use in a method to diagnose for a S. pneumoniae disorder in a human, is SP1683. Alternatively, the substantially pure or isolated antigens for use in a method to diagnose for a S. pneumoniae disorder in a human, is SP1386.

The substantially pure or isolated disease related antigen of present invention, taken in isolation or in combination (supra), can be (further) combined with at least one human immunogenic antigen of the group consisting of Zinc metalloprotease B (zmpB), GroEL, and ABC transporter for spermidine/putrescie (potD) or a fragment thereof or a substantially identical antigen for use in a diagnostic treatment to diagnose for a S. pneumoniae disorder in a human or it can be (further) combined with at least one human immunogenic antigen of the group consisting of Pneumococcal histidine antigen A (PhpA, BHV-11), Pneumococcal surface antigen A, Pneumococcal surface antigen C, Pneumococcal surface adhesin A, ORF SP_{—}0082, Fructose biphosphate aldolase, Endo-β-N-acetylglucosaminidase, IgA1 protease, Serine protease PrtA, α-enolase, Glyceraldehyde-3-phosphate dehydrogenase, DnaK, Pyruvate oxidase, ClpP protease, and Phosphoglycerate kinase, or a fragment thereof or a substantially identical antigen for use in a diagnostic treatment to diagnose for a S. pneumoniae disorder in a human. Again, in the group of antigens to diagnose, comprisese at least one, two or three of the antigens selected from the group consisting of SP_{—}0562, SP_{—}1683 and SP_{—}1386. In a particular embodiment of present invention the substantially pure or isolated disease related antigen of present invention is used in a treatment to induce an immunological memory in a human against S. pneumoniae or in a diagnostic treatment to diagnose for a S. pneumoniae disorder in a human and it is characterised in that the antigen is a peptide, protein or polypeptide.

It is also an object of the present invention to provide a substantially pure or isolated antibody that specifically binds to an antigen selected from the group consisting of the isolated, recombinant or synthetic S. pneumoniae human immunogenic antigens SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof for use in a diagnostic treatment to diagnose for a S. pneumoniae disorder in a human. In said diagnostic methods the aforementioned S. pneumoniae disease related antibodies can be used in isolation or; two or more, in particular 3, 4, 5, 6, or 7 of the disease related antibodies specific for an antigen selected the group consisting of Zinc metalloprotease C (zmpC), ABC transporters (glutamine, maltose/maltodextrin, SP_{—}1683), PTS system IIA component (mannose), pyruvate kinase, SP1290, S_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), and fragments thereof, are used in a method to diagnose for a Streptococcus pneumoniae disorder in a human. In a particular embodiment two or more, in particular 3, 4, 5, 6, or 7 of the disease related related antibodies specific for an antigen selected the group consisting of SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), and fragments thereof are used in a method to diagnose for a Streptococcus pneumoniae disorder in a human. In a particular embodiment the antibodies as used in the diagnostic method(s) are specific for a substantially pure or isolated antigen selected from the group consisting of SP_{—}0562, SP_{—}1683 and SP_{—}1386.

In a particular embodiment the antibody selectively binds to a Periplasmic Binding Protein (PBP) (in particular SP_{—}1683 or SP_{—}1386), or a fragment thereof, for use in a diagnostic treatment to diagnose for a S. pneumoniae disorder in a human. More in particular the antibody for use in a diagnostic treatment to diagnose for a S. pneumoniae disorder in a human, is specific for SP_{—}1683 or SP_{—}1386. Even more in particular the antibody for use in ₐ diagnostic treatment to diagnose for a S. pneumoniae disorder in a human, is specific for SP_{—}1683. Alternatively, the antibody for use in a diagnostic treatment to diagnose for a S. pneumoniae disorder in a human, is specific for SP_{—}1386.

The substantially pure or isolated disease related antibodies of the present invention, taken in isolation or in combination (supra), can be (further) combined with at least one antibody specific for a human immunogenic antigen of the group consisting of Zinc metalloprotease B (zmpB), GroEL, and ABC transporter for spermidine/putrescie (potD) or a fragment thereof or a substantially identical antigen for use in a diagnostic treatment to diagnose for a S. pneumoniae disorder in a human or it can be (further) combined with at least one antibody specific for a human immunogenic antigen of the group consisting of Pneumococcal histidine antigen A (PhpA, BHV-11), Pneumococcal surface antigen A, Pneumococcal surface antigen C, Pneumococcal surface adhesin A, ORF SP_{—}0082, Fructose biphosphate aldolase, Endo-β-N-acetylglucosaminidase, IgA1 protease, Serine protease PrtA, α-enolase, Glyceraldehyde-3-phosphate dehydrogenase, DnaK, Pyruvate oxidase, ClpP protease, and Phosphoglycerate kinase, or a fragment thereof or a substantially identical antigen for use in a diagnostic treatment to diagnose for a S. pneumoniae disorder in a human. In one embodiment the group of antibodies comprises antibodies selective for at least one, two or three of substantially pure or isolated antigen selected from the group consisting of SP_{—}0562, SP_{—}1683 and SP_{—}1386.

Yet another aspect of present invention is a method to screen for microbial antigens which are immunogenic in human, induce an immune response or induce an immunological memory if the microbial organism invades the human body, characterised in that 1) non-viable microbial organism delivered to severe combined immunodeficient (SCID/SCID) mice that received a natural killer depleting TMβ1 treatment (a rat monoclonal antibody (Ab) directed against the murine IL2 receptor beta chain is used for in vivo depletion of mouse natural killer cell activity) and that has been treated with human PBMC and 2) the immune response is evaluated by detecting antibodies against known human immunogens for that microbial organism and 3) immunogenic pneumococcal proteins are identified.

In a particular embodiment of the aforementioned screening method, the non-viable microbial organism is intraperotoneally (i.p.) delivered. In a further embodiment also the human PBMC is i.p. delivered. In these screening methods said human PBMC can be delivered prior to the administration of the non-viable microbial organism or it can be applied at the same time. As provided in more detail in the examples hereinafter, in one embodiment of the present invention, the immunogenic pneumococcal proteins are identified by immunoproteomic/Maldi-tof-tof analysis.

The immune response of the immunodeficient (SCID/SCID) mice is determined by detecting antibodies against the microbial organisms. Human monoclonal antibodies, having the desired specificity and the characteristics, are for instance produced by transformation of B lymphocytes obtained from peripheral blood of the SCID/SCID mice which have been injected with the killed or non viable microbial cells according to the method of present invention. B cells collected from the SCID/SCID mouse are transformed by infection with the Epstein-Barr virus and by surface antigen activation, thanks to techniques well known to those skilled in the art (Madec et al. (1996) J Immunol 156:3541-3549). The cell supernatants containing the desired antibody are identified by antigens in specific tests. Thus, the antibodies directed to the specific surface (poly)peptides or protein antigens are for example identified by reacting the supernatant with polystyrene plates coated with that antigen or with a the organism. The binding of specific antibodies is detected by addition of an anti-human IgG coupled with an enzyme. The addition of an enzyme substrate converted in a colored compound in presence of an enzyme allows to detect specific antibodies. Such methods referred to as ELISA (Enzyme-Linked Immuno-Sorbent Assays), are well known to those skilled in the art. A detailed description is available in (Current Protocols in Immunology, Chapter 2, John Wiley & Sons, Inc, 1994). The B cells producing the antibodies directed against the specific antigen are later expanded and cloned by limit dilutions. Methods of cloning are described, for example, in (Current Protocols in Immunology, Chapter 2, John Wiley & Sons, Inc. 1994).

The antibodies can also be generated and selected using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library. Phage expressing an antigen binding domain that binds the antigen of interest can be selected or identified with the selected microbial surface (poly)peptide or protein antigens, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

It is also an objective of the present invention to provide a method for in vitro diagnosis of a S. pneumoniae infection, characterised in that the presence of antibodies that specifically binds to a disease related antigen of S. pneumoniae selected from the group consisting of Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof or substantially identical antigen in a body fluid of a patient is identified by allowing said antibodies to bind with a disease related antigen of S. pneumoniae selected from the group consisting of Zinc metalloprotease C (zmpC), ABC transporters (glutamine, maltose/maltodextrin, SP_{—}1683), PTS system IIA component (mannose), pyruvate kinase, SP_{—}1290, SP_{—}0562, SP_{—}0965, SP_{—}0082 (in particular the fragments SP4 and SP17 of said SP_{—}0082), a Periplasmic Binding Protein (PBP) (in particular SP_{—}1683 or SP_{—}1386), or a fragment thereof or substantially identical antigen, and indicating said binding for instance by a label. In one embodiment of this method for in vitro diagnosis of a S. pneumoniae infection, the group of S. pneumoniae disease related antigens consists of SP_{—}0562, SP_{—}0965, SP_{—}0082 (in particular the fragments SP4 and SP17 of said SP_{—}0082), and a Periplasmic Binding Protein (PBP) (in particular SP_{—}1683 or SP_{—}1386); or a fragment of any one of said S. pneumoniae disease related antigens. More in particular the S. pneumoniae disease related antigens used in the aforementioned in vitro diagnosis method consists of a Periplasmic Binding Protein (PBP) (in particular SP_{—}1683 or SP 1386).

Further scope of applicability of the present invention will become apparent from the detailed description given herein after. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### BRIEF DESCRIPTION OF THE FIGURES

FIG. 1 Represents the % survival of Balb/c mice (n=6/group) that were immunized i.p. with 25 μg recombinant pneumococcal protein in PBS containing 1 mg/mL Alum adjuvants on day 0 and boosted on day 14. On day 28-32 these mice were challenged with S. pneumoniae serotype 3 (10⁴ CFU). Survival up to 7 days after infection (challenge) is provided.

FIG. 2 Represents the % survival of humanized SCID/SCID mice that were immunized i.p. with 25 μg rSP_{—}1386 in PBS containing 1 mg/mL Alum adjuvants (n=5), with the adjuvants (n=3), or with intact heat inactivated S. pneumoniae (serotype 3, n=3) on day 0 and boosted on day 14. On day 28 these mice were challenged with S. pneumoniae serotype 3 (10⁴ CFU). Survival up to 7 days after infection (challenge) is provided.

### DETAILED DESCRIPTION OF EMBODIMENTS OF THE INVENTION

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the F(ab′)2dimer into an Fab′ monomer. The Fab′ monomer is essentially an Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv or scFv) antibodies in which a variable heavy and a variable light chain are joined together (directly or through a peptide linker) to form a continuous polypeptide.

The “antigens” or “immunogen” refers to any substance (e.g. acterial, food, or pollen protein, or some complex carbohydrates), usually a protein, that elicits the formation of antibodies that react with it when introduced parenterally into an individual or species to which it is foreign. A protein immunogen (any substance capable of inducing an immune response) is usually composed of a large number of “antigenic determinants” or “epitopes”. Thus, immunizing an animal with a protein results in the formation of a number of antibody molecules with different specificities. The antigenicity of a protein is determined by its sequence of amino acids as well as by its conformation. Antigens may be introduced into an animal by ingestion, inhalation, sometimes by contact with skin, or more regularly by injection into the bloodstream, skin, peritoneum, or other body part.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term antibody includes antigen-binding portions of an intact antibody that retain capacity to bind S. pneumoniae protein antigen. Examples of binding include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv) ; See, e.g., Bird et al. (1988) Science 242:423-426; and Huston et al. (1988) Proc. Natl. Acad. Sci. USA 85:5879-5883). Such single chain antibodies are included by reference to the term “antibody” Fragments can be prepared by recombinant techniques or enzymatic or chemical cleavage of intact antibodies.

A bispecific antibody has two different binding specificities, see. e.g., U.S. Pat. Nos. 5,922,845 and 5,837,243; Zeilder (1999) J. Immunol. 163:1246-1252; Somasundaram (1999) Hum. Antibodies 9:47-54; Keler (1997) Cancer Res. 57:4008-4014. For example, the invention provides bispecific antibodies having one binding site for a cell surface antigen, such as human S. pneumoniae protein antigen, and a second binding site for an Fc receptor on the surface of an effector cell. The invention also provides multispecific antibodies, which have at least three binding sites. The term “bispecific antibodies” further includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (See, e.g., Holliger, P., et al. (1993) Proc. Natl. Acad. Sci. USA 90:6444-6448; Poljak, R. J., et al. (1994) Structure 2:1121-1123).

The term “human antibody” includes antibodies having variable and constant regions (if present) derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which CDR sequences derived from the germline of another mammalian species, such as a mouse, have been grafted onto human framework sequences (i.e., humanized antibodies).

The terms “monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “diclonal antibody” refers to a preparation of at least two antibodies to human S. pneumoniae protein antigen. Typically, the different antibodies bind different epitopes.

The term “oligoclonal antibody” refers to a preparation of 3 to 100 different antibodies to human S. pneumoniae protein antigen. Typically, the antibodies in such a preparation bind to a range of different epitopes.

The term “polyclonal antibody” refers to a preparation of more than 1 (two or more) different antibodies to human S. pneumoniae protein antigen. Such a preparation includes antibodies binding to a range of different epitopes.

Other antibody preparations, sometimes referred to as multivalent preparations, bind to human S. pneumoniae protein antigen in such a manner as to crosslink multiple S. pneumoniae protein antigen on the S. pneumoniae.

Cross-linking can also be accomplished by combining soluble divalent antibodies having different epitope specificities. These polyclonal antibody preparations comprise at least two pairs of heavy and light chains binding to different epitopes on S. pneumoniae.

The term “recombinant human antibody” includes all human antibodies of the invention that are prepared, expressed, created or isolated by recombinant means, such as antibodies isolated from an animal (e.g., a mouse) that is transgenic for human immunoglobulin genes (described further in Section I, below); antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterohybrid antibody” refers to an antibody having a light and heavy chains of different organismal origins. For example, an antibody having a human heavy chain associated with a murine light chain is a heterohybrid antibody. Examples of heterohybrid antibodies include chimeric and humanized antibodies, discussed supra.

The term “substantially pure” or “isolated” means an object species (e.g., an antibody or an dease related antigen of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. An isolated disease related antigen of S. pneumoniae of present invention can be substantially free of other such antigens and/or other cellular materials or chemicals.

An isolated antibody to S. pneumoniae can be substantially free of other antibodies that lack binding to S. pneumoniae and bind to a different antigen. An isolated antibody that specifically binds to an epitope, isoform or variant of S. pneumoniae may, however, have cross-reactivity to other related antigens, e.g., from other species (e.g., S. pneumoniae species homologs). Moreover, an isolated antibody of the invention be substantially free of other cellular material (e.g., non-immunoglobulin associated proteins) and/or chemicals.

“Specific binding” refers to antibody binding to a predetermined antigen. The phrase “specifically (or selectively) binds” to an antibody refers to a binding reaction that is determinative of the presence of the protein in a heterogeneous population of proteins and other biologics. Typically, the antibody binds with an association constant (Ka) of at least about 1×106 M-1 or 107 M-1, or about 108 M-1 to 109 M-1, or about 1010 M-1 to 1011 M-1 or higher, and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”.

The phrase “specifically bind(s)” or.“bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions may require a binding moiety that is selected for its specificity for a particular target antigen. A variety of assay formats may be used to select ligands that are specifically reactive with a particular protein. For example, solid-phase ELISA immunoassays, immunoprecipitation, Biacore and Western blot are used to identify peptides that specifically react with S. pneumoniae. Typically a specific or selective reaction will be at least twice background signal or noise and more typically more than 10 times background

The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin gene locus can be identified by comparison to germline DNA; a rearranged locus has at least one recombined heptamer/nonamer homology element.

The term “unrearranged” or “germline configuration” in reference to a V segment refers to the configuration wherein the V segment is not recombined so as to be immediately adjacent to a D or J segment.

Manuals are available for the many skilled in the art for achieving such antibodies or rearranged antibodies. An overview is provided in the recent work, Handbook of Therapeutic Antibodies Edited by Stefan Dübel, Wiley-VCH Verlag GmBH & Co, KGaA.

The term “nucleic acid” is intended to include DNA molecules and RNA molecules. A nucleic acid can be single-stranded or double-stranded.

The term “substantially identical,” in the context of two nucleic acids or polypeptides refers to two or more sequences or subsequences that have at least about 80%, about 90, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. In case of antibodies, any two antibody sequences can only be aligned in one way, by using the numbering scheme in Kabat (see hereunder). Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention.

The phrase “selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

The phrase “stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found An extensive guide to the hybridization of nucleic acids is found in e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID P ROBES, Part I. Theory and Nucleic Acid Preparation, Tijssen, ed. Elsevier, N.Y. (1993).

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include hybridization in a buffer of 40% formamide, 1 M NaCl, 1% SDS at 37° C.

However, the selection of a hybridization format is not critical--it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. see, e.g., Sambrook, Tijssen and Ausubel. The nucleic acid sequences of the invention and other nucleic acids used to practice this invention, whether RNA, cDNA, genomic DNA, or hybrids thereof, may be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as,. e.g., subcloning into expression vectors, labeling probes, sequencing, and hybridization are well described in the scientific and patent literature, see, e.g., Sambrook, Tijssen and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites and the like), from either cDNA, genomic or mixtures may be mutated, thereof in accordance with standard techniques to provide gene sequences. For coding sequences, these mutations, may affect amino acid sequence as desired. In particular, DNA sequences substantially homologous to or derived from such sequences described herein are contemplated (where “derived” indicates that a sequence is identical or modified from another sequence).

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e. g., replication defective retroviruses, adenoviruses and adeno-associated viruses), which serve equivalent functions.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact; be identical to the parent cell, but are still included within the scope of the term “host cell” as used herein.

The term “minilocus transgene” refers to a transgene that comprises a portion of the genomic immunoglobulin locus or on the locus of the selected disease antigen having at least one internal (i.e., not at a terminus of the portion) deletion of a non-essential DNA portion (e.g., intervening sequence; intron or portion thereof) as compared to the naturally-occurring germline Ig locus.

A “label” is a composition detectable by spectroscopic, photochemical, biochemical, immunochemical, or chemical means. For example, useful labels include 32P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

The term “sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

The terms “T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

The term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Components of an immune response may be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen A; et al., 1995, Immunity 2 (4): 373-80), (3) antigen presenting cells can be incubated with whole protein antigen and the presentation of that antigen on MHC detected by either T lymphocyte activation assays or biophysical methods (Harding et al., 1989, Proc. Natl. Acad. Sci., 86: 4230-4), (4) mast cells can be incubated with reagents that cross-link their Fc-epsilon receptors and histamine release measured by enzyme immunoassay (Siraganian, et al., 1983, TIPS 4: 432-437).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., 1988, Blood 72: 1310-5); (3) the proliferation of peripheral blood mononuclear cells in response to mitogens or mixed lymphocyte reaction can be measured using 3H-thymidine; (4) the phagocitic capacity of granulocytes, macrophages, and other phagocytes in PBMCs can be measured by placing PMBCs in wells together with labeled particles (Peters et al., 1988); and (5) the differentation of immune system cells can be measured by labeling PBMCs with antibodies to CD molecules such as CD4 and CD8 and measuring the fraction of the PBMCs expressing these markers.

Except when noted, the terms “patient” or “subject” are used interchangeably and refer to a human patients.

The terms “treating” or “treatment” include the administration of the compounds or agents of the present invention to prevent or delay the onset of the symptoms, complications, or biochemical indicia of a disease, alleviating the symptoms or arresting or inhibiting further development of the disease, condition, or disorder (e.g., autoimmune disease). Treatment may be prophylactic (to prevent or delay the onset of the disease, or to prevent the manifestation of clinical or subclinical symptoms thereof) or therapeutic suppression or alleviation of symptoms after the manifestation of the disease.

An “immunostimulant,” or “immunostimulatory” molecule or domain or the like, herein refers to a molecule or domain, etc. which acts (or helps to act) to stimulate or elicit an immune response or immune action in a subject (either cellular or humoral or both). Typical examples of such molecules include, but are not limited to, e.g., cytokines and chemokines. Cytokines act to, e.g., stimulate humoral and/or cellular immune responses. Typical examples of such include, e.g., interleukins such as IL-2, IL-12, etc. Chemokines act to, e.g., selectively attract various leukocytes to specific locations within a subject. They can induce both cell migration and cell activation. Common examples of chemokines include, e.g., RANTES, C-X-C family molecules, II-8, mip1α, mip1β, etc. For further information, see, e.g., Arai, K. et al, 1990, “Cytokines: coordinators of immune and inflammatory responses” Annu Rev Biochem 59:783+; Taub, 1996 “Chemokine-Leukocyte Interactions. The Voodoo That They Do So Well” Cytokine Growth Factor Rev 7:355-76.

A “disease related antigen” refers to an antigenic protein, peptide or polypetids or the nucleic acid that encodes such, or combination of any of such, which arises or is present in a subject due to a pneumoccal infection in particular of S. peumoniae infection.

“Vaccinating Agent” refers to a composition which is capable of stimulating a protective immune response within the host which receives the vaccinating agent. The vaccinating agent may be either protein, or, DNA-based (e.g., a gene delivery vehicle). Within further aspects, a prokaryotic host may be generated to be a vaccinating agent, and designed to express an immunogenic polypeptide or multivalent construct of the present invention (see, e.g., U.S. application Ser. No. 07/540,586).

“Gene delivery vehicle” refers to a recombinant vehicle, such as a recombinant viral vector, a nucleic acid vector (such as plasmid), a naked nucleic acid molecule such as genes, a nucleic acid molecule complexed to a polycationic molecule capable of neutralizing the negative charge on the nucleic acid molecule and condensing the nucleic acid molecule into a compact molecule, a nucleic acid associated with a liposome (Wang et al., PNAS 84: 7851, 1987), a bacterium, and certain eukaryotic cells such as a producer cell, that are capable of delivering a nucleic acid molecule having one or more desirable properties to host cells in an organism.

Streptococcus pneumoniae is an encapsulated gram positive bacterium that belongs to the commensal flora of the human upper respiratory tract. It is a major cause of bacterial otitis media, pneumoniae, meningitis, and bacteraemia, causing great morbidity and mortality throughout the world. Especially critical ill patients that are artificially ventilated in a critical care unit, young children, the elderly and people with an underlying disease are vulnerable for infection with S. pneumoniae. The recent emergence of penicillin- and multi-drug resistant S. pneumoniae strains complicates treatment and increases the burden on public health systems. An effective means to control pneumococcal disease relies on preventive strategies such as vaccination (Bogaert D., et al. Lancet Infect Dis. 4: 144-154 and Barocchi M. A., S. et al. Vaccine 25: 2963-2973°.

The capsule was the first recognized virulence factor of S. pneumoniae. It is composed of polysaccharides and protects the bacteria from phagocytosis. Ninety different capsular serotypes have been identified. A polyvalent mixture of 23 capsular serotypes [Pneumo 23® (Sanofi-Pasteur MSD)] was one of the first licensed pneumococcal vaccines. The serotypes contained within this vaccine account for 90% of the serotypes causing serious pneumococcal disease in industrialized countries (Recommendations of the Advisory Committee on Immunization Practices (ACIP). 1997, 46 (RR-8), 1-24). The vaccine induces serotype-specific antibodies which provide host protection by induction of opsonophagocytosis (Bogaert D., et al. 2004. Lancet Infect Dis. 4: 144-154). Although the vaccine elicits a relatively good specific antibody response in most healthy adults, it elicits an inefficient antibody response in children less than two years of age and in immuno-compromised individuals (Koskela M., et al. Pediatr Infect Dis. 5(1): 45-50 and Kroon F. P. et al. Vaccine 18(5-6): 524-530.). Moreover, the vaccine does not induce immunological memory (Bogaert D., et al. Vaccine 22: 2209-2220.). To overcome these problems, vaccine manufacturers have developed pneumococcal conjugate vaccines in which pneumococcal polysaccharides are covalently coupled to a protein carrier in order to elicit a T cell dependent immune response. In Prevenar® (Wyeth), seven different polysaccharides, which are dominantly found among pediatric patients in the United States, have been conjugated to a detoxified mutant of diphtheria toxin (Tai S. S. 2006. et at Critical Reviews in Microbiology 32: 139-153.). It is efficacious against invasive pneumococcal disease in children less than two year old (Bogaert D., et al. Vaccine 22: 2209-2220). Nine-valent (Wyeth) and 13-valent (GSK and Sanofi-Pasteur) conjugated vaccines are in trial. The high manufacturing costs of the conjugated vaccines and the increased presence of serotypes that are not contained in the vaccine force the search for new approaches, such as the development of protein-based pneumococcal vaccines (Tai S. S. 2006. et al. Critical Reviews in Microbiology 32: 139-153).

A protein-based pneumococcal vaccine approach has several advantages. First, the production of protein vaccines is expected to be cheap and therefore within the reach of developing countries (Swiatlo E., and D. Ware. 2003. FEMS Immunol. Med. Microbiol. 38: 1-7). Second, a protein-based vaccine is likely to give rise to immunological memory and to elicit protection in all age-groups, including children younger than two years of age. Finally, if highly conserved proteins or protein epitopes are used as vaccine components, broad and serotype independent protection can be expected (Barocchi M. A., et al. 2007. Vaccine 25: 2963-2973).

Several strategies have been employed to identify proteins which contribute to pneumococcal virulence and which are capable to induce antibody production and host protection.

Because of their location, pneumococcal surface proteins are considered potential vaccine candidates. With the increasing availability of multiple complete genome sequences [strain TIGR4 (serotype 4), strain D39 (serotype 2), and laboratory strain R6 (an avirulent, unencapsulated derivative of strain D39)] and powerful bioinformatics tools, efficient computer-aided search strategies have been developed to identify proteins that contain certain sequence motifs typical for secreted proteins or for a surface location from the genome of S. pneumoniae (Tai S. S. 2006. Critical Reviews in Microbiology 32: 139-153 and Lanie J. A., et al. 2007. J. Bacteriol. 189(1): 38-51.). Three clusters of classical surface proteins can be distinguished: lipoproteins (LXXC amino acid motive, covalently linked to the cell membrane), choline-binding proteins (C-terminal repeats of 20 amino acids, electrostatically bound to phosphocholine or (lipo) teichoic acid) and cell wall anchored proteins (LPXTG amino acid motive, covalently bound to cell wall peptidoglycan) (Rigden D. J., et al. 2003. Crit. Rev. Biochem. Mol. Biol. 38(2): 143-168.). Besides, non-classical surface proteins, also known as moonlighting proteins, lacking a classical leader peptide and membrane-anchoring motifs have as well been identified on the pneumococcal surface (Bergmann S. and S. Hammerschmidt. 2006. Microbiol. 152: 295-303.).

Signature-tagged mutagenesis techniques and genetically modified S. pneumoniae have been used to identify pneumococcal proteins that contribute to the bacterial virulence (Polissi A., et al. 1998. Infect. Immun. 66(12): 5620-5629.; Lau G. W., 2001. Mol. Microbiol. 40(3): 555-571 and Oggioni M. R., 2003. Mol. Microbiol. 49(3): 795-805.).

Immunoscreening methods with sera obtained from immunized mice, children attending day-care, healthy adults and/or human convalescent sera have been used to identify immunogenic pneumococcal proteins (Ling E., G. et al; 2004. Clin. Exp. Immunol. 138: 290-298; McCool T. L., et al. 2002. J. Exp. Med. 195(3): 359-365; Holmlund E., et al. 2006. Vaccine. 24: 57-65.). The protective capacity of these antigenic proteins alone or in combination has mainly been evaluated in animal models using recombinant proteins. Passive immunization of mice with sera obtained from healthy human volunteers after immunization with recombinant pneumococcal protein has been reported in one study (Briles D. E., et al. 2000. J. Infect. Dis. 182: 1694-1701).

Although a lot of information is available on immunogenic pneumococcal proteins, only few proteins have been tested in clinical trials. The ideal protein-based pneumococcal vaccine formulation, however, has not yet been discovered.

In the present study we used an immuno-proteomics approach to identify immunogenic proteins in S. pneumoniae. Humanized severe combined immunodeficient (SCID) mice were immunized with inactivated S. pneumoniae. Immunogenic proteins from S. pneumoniae were identified and characterized.

SCID/SCID mice were transplanted with human mononuclear cells and immunized with inactivated S. pneumoniae serotype 3. Two weeks after immunization, serum was obtained. The serum was used in Western blotting analyses after two-dimensional separation of an extract of S. pneumoniae. The proteins to which there was reactivity were excised and identified by mass spectrometry. The immunogenic proteins we identified included pneumococcal histidine protein A (PhtA), pneumococcal surface protein A (PspA), PspC, pneumococcal surface adhesion A (PsaA), open reading frames (SP_{—}0082, SP_{—}1290, SP_{—}0562 and SP_{—}1683), fructose biphosphate aldolase (FBA), endo-β-N-acetylglucosaminidase, zinc metalloprotease B (zmpB), zmpC, IgA1 protease, serine protease PrtA, α-enolase, glyceraldhyde-3-phosphate dehydrogenase (GAPDH), ABC transporters (Maltose/maltodextrin, glutamine, spermidine/putresine), PTS system IIA component (mannose), DnaK, GroEL, pyruvate oxidase, phosphoglycerate kinase, and pyruvate kinase. They are summarized in Table I and can be classified as histidine triad proteins, choline binding proteins, adhesins, proteins involved in the degradation of the extracellular matrix, transporters, stress proteins, proteins involved in various physiological processes, and hypothetical proteins.

Histidine Triad Proteins

PhtA is a histidine triad protein that has been found to possess complement C3 degradation activity. This protein is able to induce antibodies capable of protecting mice against pneumococcal sepsis and death (Hamel J., N. et al. 2004. Infect. Immun. 72(5): 2659-2670). It is currently tested as a candidate pneumococcal vaccine in clinical trials (phase II) (Barocchi M. A., et al. 2007. Vaccine 25: 2963-2973. and Zhang Y., et al. 2001. Infect. Immun. 69(6): 3827-3836).

Choline Binding Proteins

The choline binding proteins PspA and PspC are considered realistic vaccine candidates, because of their location on the surface of the microorganism and of their biological functions (Rigden D. J., et al. 2003. Crit. Rev. Biochem. Mol. Biol. 38(2): 143-168.). They interfere with the complement system (PspA, PspC), have anti-bactericidal activity (PspA), and support colonization (PspC) (10). Although PspA and PspC are currently evaluated as candidate vaccines in clinical and pre-clininical trials (Balachandran P., et al. 2002. Infect. Immun. 70(5): 2526-2534), they have some disadvantages. Psp proteins are diverse with variable molecular sizes (Lannelli F., et al. 2002. Gene 284: 63-71 and Beall B., et al. 2000. J. Clin. Microbiol. 38: 3663-3669.). Moreover, PspA shows cross-reactivity with human myoglobin (Barocchi M. A., et a. Vaccine 25: 2963-2973).

Adhesins

Pneumococcal surface adhesin A (PsaA) is currently evaluated as a vaccine candidate in clinical studies (Briles D. E., et al. 2000. Infect. Immun. 68(2): 796-800). PsaA is undetectable on the bacterial surface and anti-PsaA antibodies are probably only important in protection against carriage (Briles D. E., et al. 2000. Infect. Immun 68(2): 796-800).

Open reading frame (ORF) SP_{—}0082 encodes a surface protein with a LPXTG anchoring motive. It contains four copies of a novel conserved streptococcal surface repeat domain (Rigden D. J., et al. Crit. Rev. Biochem. Mol. Biol. 38(2): 143-168). ORF SP_{—}0082 has been suggested as a virulence and physiologically important gene (Lanie J. A., et al. 2007. J. Bacteriol. 189(1): 38-51). Bumbaca et al. characterized ORF SP_{—}0082 as a fibronectin binding surface adhesin (Bumbaca D. et al 2004. OMICS 8(4): 341-356). After screening a S. pneumoniae D39 library with sera obtained from infected individuals, ORF spr_{—}0075 of serotype 2 was identified. Comparative analysis demonstrated that this ORF was identical to ORF SP_{—}0082 of serotype 3 and well preserved among other strains (type 19F, 6B, 4, 23F) (Beghetto E., et al. 2006. FEMS Microbiol. Lett. 262: 14-21). Another fibronectin binding pneumococcal protein, pneumococcal adherence and virulence factor A (PavA) was found as a non-significant hit. This protein has been shown critical for invasive diseases (Pracht D., et al. 2005. Infect. Immun. 73: 2680-2689). Anti-PavA antibodies have been found in human convalescent-phase sera and were protective in a mouse model of lethal sepsis (Wizemann T. M., et al. 2001. Infect. Immun. 69(3): 1593-1598.).

FBA is a well known cytoplasmatic glycolytic enzyme. Blau et al. identified FBA as a cell wall-localized lectin that acts as a S. pneumoniae adhesin via the human Flamingo cadherin receptor.

Moreover, a peptide comprising a putative FBA-binding region of the Flamingo cadherin receptor inhibited nasopharyngeal and lung colonization in a mouse model (Blau K., et al. 2007. J. Infect. Dis. 195: 1828-1837). Ling et al. proposed FBA as a candidate for a pneumococcal vaccine because (i) anti-FBA antibodies were present in sera obtained from children attending day-care centres and healthy adult volunteers, (ii) mouse antibodies elicited to recombinant FBA were cross-reactive with several genetically unrelated strains of different serotypes and conferred protection to respiratory challenge with virulent pneumococci, and (iii) pneumococcal FBA does not have a human homologue (Ling E., et al. 2004. Clin. Exp. Immunol. 138: 290-298).

Proteins Involved in the Degradation of the Human Extracellular Matrix

Different pneumococcal proteins are involved in the degradation of the human extracellular matrix thereby facilitating colonization, adherence and eventually invasion. Especially extracellular enzyme systems involved in the metabolism of polysaccharides and hexosamines are able to degrade the host polymers, including mucins, glycolipids, and hyaluronic acid (Tettelin H., et al. 2001. Science 293: 498-506.).

Endo-β-N-acetylglucosaminidase, which contains a LPXTG anchoring motive, cleaves the di-N-acetylchitobiose structure in asparagine-linked oligosaccharides (Muramatsu H. et al. 2001. J. Biochem. 129: 923-928). Antibodies against this enzyme were present in human convalescent-phase sera and were protective in a mouse model of lethal sepsis (Wizemann T. M., et al. 2001. Infect. Immun. 69(3): 1593-1598 and Zysk G., 2000. Infect. Immun. 68(6): 3740-3743).

S. pneumoniae strains possess at least three large extra-cellular or surface-associated zinc metalloproteases; ZmpB, ZmpC, and IgA1-protease. Antibodies against these three zinc metalloproteases were identified in our humanized-SCID model. ZmpB has been found antigenic in mice (Beghetto E., et al. 2006. FEMS Microbiol. Lett. 262: 14-21). The substrate of ZmpB is still unknown. ZmpC facilitates host invasion by cleaving human matrix metalloproteinase 9, a protease that cleaves gelatine and collagen (Oggioni M. R., et al. 2003. Mol. Microbiol. 49(3): 795-805). ZmpC has been shown to participate in pneumococcal pathogenicity in an experimental murine model of intranasal challenge and sepsis (Chiavolini D., et al. 2003. BMC Microbiol. 3: 14-25). IgA1 protease cleaves the hinge region of human IgA, the predominant immunoglobulin class present on mucosal membranes and thereby facilitates adherence of the bacteria (Bogaert D., Ret al. 2004. Lancet Infect Dis. 4: 144-154). Different studies have shown that IgA1 protease is a major S. pneumoniae antigen (Audouy S. A., et al. 2007. Vaccine 25(13): 2497-2506, Weiser J. N., et al. 2003. Proc. Natl. Acad. Sci. USA. 100: 4215-4220). It has been suggested that an inactive mutant can be a possible candidate for an anti-pneumococcal vaccine (Romanello V., et al. 2006. Protein Expr. Purif. 45: 142-149, McCool T. L., et al. 2003. Infect. Immun. 71(10): 5724-5732).

We also found antibodies against PrtA, which is another surface-exposed serine protease with an LPXTG anchoring motive. PrtA knockout bacteria have been shown to be attenuated in an intraperitoneal mouse infection model (Bethe G., R. et al. 2001. FEBS Microbiol. Lett. 205: 99-104). Antibodies against this serine protease were present in human convalescent-phase sera and were protective in a mouse model of lethal sepsis (Wizemann T. M., et al. 2001. Infect. Immun. 69(3): 1593-1598).

Glyceraldehyde-3-phosphate dehydrogenase (GAPDH) and a-enolase are plasminogen-binding proteins displayed on the bacterial cell surface (Bergmann S., et al. 2004. Infect. Immun. 72(4): 2416-2419.: Bergmann S, et al. 2001. Mol. Microbiol. 40(6): 1273-1287.). Surface-bound plasmin activity has been shown to be associated with potential degradation of the extracellular matrix, dissolution of fibrin, and pneumococcal transmigration (Bergmann S., et al. 2005. Thromb. Haemost. 94(2): 304-311). Both glycolytic enzymes are non-classic proteins lacking known signal peptides and membrane anchoring motifs. We found antibodies against these two plasminogen binding proteins. Furthermore, GAPDH is antigenic in children attending day-care centers and in healthy adult volunteers and is protective in mice intranasally challenged with virulent pneumococci (Ling E., et al. 2004. Clin. Exp. Immunol. 138: 290-298). α-Enolase was found immunogenic in sera from patients with pneumococcal disease (Whiting G. C. et al. 2002. J. Med Microbiol. 51: 837-843).

Transporters

S. pneumoniae has a wide substrate utilization range of sugars and substituted nitrogen compounds. A large number of membrane located transporters have been identified (Rigden D. J. et al. 2003. Crit. Rev. Biochem. Mol. Biol. 38(2): 143-168). Sugar transporters of S. pneumoniae primarily consist of phosphoenolpyruvate-dependent phosphotransferase system (PTS) transporters and ATP-binding cassette (ABC) transporters (Tettelin H., et al. 2001. Science 293: 498-506.).

In this study we detected antibodies against PTS system IIA component involved in the uptake of mannose, and against an ABC sugar transporter necessary for the uptake of maltose/maltodextrin.

Moreover, we also detected antibodies against the sugar-binding protein of the sugar ABC transporter (SP_{—}1683).

We also found antibodies to other transporters, specifically to ABC transporters for glutamine (glnQ), manganese (PsaA) and spermidine/putrescine (potD). Polissi et al. classified glnQ and potD as medium virulent pneumococcal genes in a mouse septicaemia model (Polissi A., et al. 1998. Infect. Immun. 66(12): 5620-5629). Previous studies have shown that a potD mutation in a mouse-virulent capsular type 3 strain significantly attenuated the natural history of infection in a murine model, indicating a role for PotD in pneumococcal pathogenesis (Ware D., et al. 2006. Infect. Immun. 74: 352-361). Shah P. et al. showed that antibodies to recombinant PotD conferred protection against invasive pneumococcal disease (Shah P. and E. Swiatlo. 2006. Infect. Immun. 74(10): 5888-5892). The authors suggested that this protein should be studied further as a potential vaccine candidate for protection against invasive pneumococcal infections (Shah P. and E. Swiatlo. 2006. Infect. Immun. 74(10): 5888-5892).

We also found antibodies to the ABC transporter for spermidine (SP_{—}1386). Like the sugar ABC transporter (SP_{—}1683) this ABC spermidine transporter is a member of the PBPb Super-family. These proteins have a conserved PBPb domain and are known as Bacterial periplasmic transport systems that use membrane-bound complexes and substrate-bound, membrane-associated periplasmic binding proteins (PBPs) to transport a wide variety of substrates, such as, amino acids, peptides, sugars, vitamins and inorganic ions. PBPs have two cell-membrane translocation functions: bind substrate, and interact with the membrane bound complex. These proteins were found to be highly effective in inducing an active immunization in the mouse models hereinafter. In SP_{—}1386 the PBPb domain is allocated from amino acids 1-356 of the sequence provided in Table XIII below. For SP_{—}1683 the PBPb domain is allocated from amino acids 65-350 of the sequence provided in Table V below

Stress Proteins

Heat shock proteins (HSP) are extremely well conserved. They are induced upon infection and under various stress conditions, such as starvation, exposure to free radicals, and heat shock (Hendrick J. P. and F.-U. Hartl. 1993. Ann. Rev. Biochem. 62: 349-384). They can be classified as HSP100, HSP70, HSP60, and small molecular weight HSP families. HSPs play a pivotal chaperone role in folding of native and denatured proteins and promote cell protection and survival (Hendrick J. P. and F.-U. Hartl. 1993. Ann. Rev. Biochem. 62: 349-384). Evidence is now accumulating that HSPs are major antigens of various pathogens.

In our study we found antibodies against members of HSP 100 (e.g. Clp), HSP70 (e.g. DnaK), and HSP60 (e.g. GroEL). Hamel J. et al. showed that members of the HSP70 and HSP60 families are immunogenic in mice after subcutaneous injection of heat inactivated S. pneumoniae (Hamel J., D. Martin and B. B. Brodeur. 1997. Microbial pathogenesis 23: 11-21).

Antibodies against DnaK have been found in sera obtained from children attending day-care centers, healthy adult volunteers and human convalescent-phase sera (Ling E., et al. 2004. Clin. Exp. Immunol. 138: 290-298). Kolberg J. et al. found that DnaK did not induce a human antibody response during infection (Kolberg J., et al. 2000. FEMS Immunol. Med. Microbiol. 29: 289-294). Moreover, anti-DnaK antibodies were not protective in mice (Ling E., et al. 2004. Clin. Exp. Immunol. 138: 290-298., Zysk G., Ret al. 2000. Infect. Immun. 68(6): 3740-3743). Besides fulfilling a chaperone function the HSP100/caseinolytic protease family (Clp) is also involved in proteolysis, removing damaged and denatured proteins (Schirmer E. C., et al. 1996. Trends Biochem. Sci. 21: 289-296.). Proteolysis by Clp requires a serine-type peptidase ClpP subunit and a regulatory ATPase subunit (Kwon H. Y., et al. 2003. Infect. Immun. 71(7): 3757-3765). We found antibodies against the serine-type peptidase ClpP subunit. There’ is evidence that the cytoplasmatic ClpP is translocated to the cell wall after heat shock. A clpP mutant failed to colonize the murine nasopharynx or to invade the murine lungs. Mice immunized with ClpP exhibited a strong, specific antibody response which protected the mice from fatal pneumococcal challenge (Kwon H.-Y., et al. 2004 Infect. Immun. 72(10): 5646-5653). Finally, Cao et al. showed that antibodies to a mixture of PspA, PspC and ClpP can enhance protection against pneumococcal infection in mice (Cao J. et al. 2007. Vaccine 25(27): 4996-5005).

Proteins Involved in Physiological Processes

In addition to antibodies against the above-mentioned glycolytic enzymes we also detected antibodies against phosphoglycerate kinase and pyruvate kinase. Ling et at detected antibodies against phosphoglycerate kinase in sera from children attending day-care centers and healthy adult volunteers (Ling E., et al. 2004 Clin. Exp. Immunol. 138: 290-298). Pyruvate oxidase is involved in the production of H₂O₂, which promotes pneumococcal carriage (by inhibiting growth of other common pathogens) (Hoskins J., et al. 2001. J Bacteriol. 183(19): 5709-5717) and slows ciliary beating (LeMessurier K. S., et al. 2006. Microbiol. 152: 305-311). S. pneumoniae deficient in pyruvate oxidase (spxB) showed reduced virulence in animal models for nasopharyngeal colonisation, pneumoniae and sepsis (Spellerberg B., et al. 1996. Mol Microbiol. 19(4): 803-813). Lanie et al. determined spxB as a gene that contributed to pneumococcal virulence (Lank J. A., et al. 2007. J. Bacteriol. 189(1): 38-51). Ling et al. showed the presence of anti-pyruvate oxidase antibodies in sera from children attending day-care centers and healthy adult volunteers (Ling E., et al.2004. Clin. Exp. Immunol. 138: 290-298.).

Hypothetical Proteins

Antibodies against hypothetical proteins SP_{—}0562, SP_{—}0965, SP_{—}0082 and SP_{—}1290 were detected. The ORF corresponding with these hypothetical proteins can be found in various pneumococcal serotypes: TIGR4, SpD_{—}0488 (D39) and Spr_{—}0486 (R6); SpD_{—}1145 (D39) and Spr_{—}1169 (R6) for SP_{—}0562 and SP_{—}1290; respectively. ORF_{—}0562 encodes an uncharacterized protein and contains a domain (DUF1858) with unknown function but which can be found in various bacterial proteins. ORF_{—}1290 encodes a protein with a predicted phosphohydrolase function (http://www.ncbi.nlm.nih.gov/BLAST). The hypotetical protein SP_{—}0082 has been described as Cell wall surface anchor family protein with a Molecular Weight 90924 Da and protein length 857 AA. The UniProtKB/TrEMBL entry name is Q97T70_STRPN and primary accession number Q97T70. It has been described in Tettelin H., et al. RL Science 293:498-506(2001). The hypotetical protein SP_{—}0965 has been described as Putative endo-beta-N-acetylglucosaminidase with a Molecular Weight 76469 Da and protein length 658 AA. The UniProtKB/TrEMBL entry name is LYTB_STRPN and primary accession number P59205. It has been described in Tettelin H., et al. RL Science 293:498-506(2001).

### Examples

The following examples are offered by way of illustration, and not by way of limitation.

Using a humanized SCID/SCID mice model and a proteomics approach a whole array of pneumococcal proteins is described that are immunogenic for the human immune system after immunization with intact heat-inactivated S. pneumoniae. Humanized SCID/SCID mice were immunized with heat-inactivated S. pneumoniae type 3. Serum was obtained after two weeks and used in Western blotting analysis after two-dimensional separation of a S. pneumoniae extract. Proteins that reacted with serum were identified by Maldi-Tof-Tof analysis. Twenty six proteins were recognized as immunogenic. Some of them (PhtA, PspA, PsaA, PspC, ORF SP_{—}0082, endo-β-N-acetylglucosaminidase, IgA1 protease, serine protease PrtA, α-enolase, DnaK, FBA, GAPDH, pyruvate oxidase, and phosphoglycerate kinase) had already been described to elicit antibodies in infected and/or healthy individuals. Others [zmpB, GroEL, ABC transporter (spermidine) and ClpP Protease] had been described to be immunogenic (and possible protective) in mice models. A final group consisted of proteins which had not yet been reported as immunogenic after infection with S. pneumoniae. It included zmpC, ABC transporters (glutamine, maltose/maltodextrin, SP_{—}1683), PTS system IIA component (mannose), pyruvate kinase, and the hypothetical proteins SP_{—}1290 and SP_{—}0562. Some of these proteins are known to contribute to pneumococcal virulence.

Materials and Methods

### Example 1

### Mice

Six to eight week old SCID/SCID mice on a BALB/c background were kindly provided by Jan Mertens, Rega institute, Catholic University Leuven, Belgium. These SCID/SCID mice were kept in sterilized plastic cages and were given sterilized tap water and sterilized pelleted food. The SCID/SCID mice were held in a room with 12 h/12 h light/dark cycle. The SCID/SCID mice were tested for leakiness by analyzing the level of mouse IgG antibodies according to a previously described ELISA (Steinsvik T. E., et al. 1995. Scan. J. Immunol. 42: 607-616). Only SCID/SCID mice with IgG concentrations less than 2 μg/mL were used in the experiments. Approval of the study was granted by the local Ethics Committee of the Catholic University Leuven.

### Example 2

### Preparation of Intact Heat-Killed S. pneumoniae

S. pneumoniae (a kind gift of Prof. Verhaegen J, Laboratory medicine, Universal Hospitals Leuven, Belgium) was grown in Todd-Hewitt broth to mid log phase at 37° C. in a CO₂ incubator. Bacteria were inactivated at 60° C. for 90 minutes. Inactivation was confirmed by blood agar culture. Bacteria were collected by centrifugation (20 minutes, 3000 g) and the pellet was washed three times with sterile phosphate buffered saline (Gibco BRL, Life Technologies LTD. Paisley, Scotland). Bacterial stock, containing 10⁹ CFU, was divided into aliquots and frozen at −80° C. until immunization.

### Example 3

### Transferring Human Peripheral Blood Mononuclear Cells (PBMC) to SCID/SCID Mice

Peripheral blood buffy coat from healthy blood donors (n=4) was obtained from the Blood Transfusion Centre of the Red Cross Leuven. Human PBMC were prepared by density gradient centrifugation on Lymphoprep (Axis-shield Poc AS) and analyzed by flow cytometry (BD Biosciences). One day before transferring human PBMC, the SCID/SCID mice received TMβ1 (a kind gift of Prof. Waer M., Experimental transplantation, Catholic University Leuven, Belgium), a rat monoclonal antibody recognizing the mouse IL-2 receptor beta-chain, by injection intraperitoneal (i.p.) to improve the survival and functionality of the transplant (Tournoy K. G., Set al. 1998. Eur. J. Immunol. 28: 231-239). 70 10⁶ human PBMC were dissolved in phosphate buffered saline and injected i.p. into SCID/SCID mice. The mice were immunized i.p. with 2 10⁸ CFU heat-killed S. pneumoniae serotype 3 on the same day. Fourteen days later, blood was drawn by heart puncture in isofluran (Schering-Phough Animal Health, Harefield, Uxbridge, Middlesex, United Kingdom) anesthetized mice. Mice were euthanized after isofluran inhalation by cervical dislocation.

To evaluate the immune response to heat inactivated S. pneumoniae serotype 3, IgM and IgG antibodies to capsular-polysaccharide serotype 3 and PspA were measured by ELISA as previously described (Moens L., et al.2007. J. Leukoc. Bio. 82(3): 638-644)

### Example 4

### 2D Gel Electrophoresis and Western Blotting

A S. pneumoniae serotype 3 protein extract was prepared by sonication as described by Encheva et al. (Encheva V., et al. 2006. Proteomics 6(11): 3306-3317). The 2-D clean-Up kit (GE Healthcare Bio-sciences AB, Uppsala, Sweden) was applied on the S. pneumoniae serotype 3 protein extract. IPG strips ranging from pH 4 to 7 (GE Healthcare Bio-sciences AB (Uppsala, Sweden) were rehydrated overnight using 250 μg of S. pneumoniae serotype 3 protein extract. After one dimensional iso-electric focusing on a Multiphor II Electrophoresis System according to the manufacturer's instructions, the IPG strips were equilibrated in equilibration solution I [0.05 M Tris-HCl, pH 6.8 containing 6 M urea, 35 mM SDS, 30% (v/v) glycerol and 0.25% (w/v) DTT] and solution II [0.05 M Tris-HCl, pH 6.8 containing 6 M urea, 35 mM SDS, 30% (v/v) glycerol, 0.45% (v/v) jodoacetamide and bromophenol blue] for 15 min each. Equilibrated strips were loaded on a 12.5% SDS polyacrylamide gel for separation in the second dimension according to the manufacturer's instructions. Thereafter, the proteins were either transferred on a polyvinylidene difluoride (PVDF) membrane (Hybond-P, GE Healthcare Bio-sciences AB) by electroblotting using a NovaBlot apparatus or visualized by Coomassie staining (GE Healthcare Bio-sciences AB). Membranes were consecutively treated with 5% (w/v) bovine serum albumin for 1 h, with Hu-SCID mice serum (dilution 1:250) overnight, with goat anti-human IgG (dilution 1:5000) for 1 h, and with horseradish peroxidase conjugated rabbit anti-goat IgG (dilution 1:5000, DakoCytomation, Glostrup, Denmark) for 45 min. All antibodies were diluted in trissaline buffer (TSB containing 10 mM Tris-HCl, 150 mM NaCl and 0.1% Triton; pH 7.6). Intermittent washing steps were performed in TSB (3×10 min). Finally, 0.7 mM 3.3′diamino-benzidinetetrahydrochloride containing 0.1% H₂O₂ in TBS was added as a substrate to detect protein-antibody interactions after 5 min of incubation. Coomassie stained gels were used to excise the corresponding visualized spots. 2D gel electrophoresis and Western blotting experiments were performed in triplet to confirm the identity of the excised protein spots. Identification of the excised spots was performed by Maldi-Tof-Tof analysis [MASCOT ion MS/MS search engine] (Perkins D. N., et al. 1999. Electrophoresis 20: 3551-3567).

### Example 5

### Protein Identification by MALDI-TOF/TOF

Gel pieces containing the protein of interest were washed with HPLC grade water, dried in a Speed Vac (Savant) and digested overnight at 37° C. with 10 μl of 25 ng/μl trypsin (sequence grade) in 200 mM ammonium bicarbonate. The resulting peptide mixture was subjected to a C18 clean-up (ZipTip) and analyzed by a MALDI-TOF/TOF (Applied Biosystems 4800 Proteomics Analyzer) in the presence of α-cyano 4-hydrocinnamic acid (HPLC grade). Tandem MS data were submitted to the MASCOT search engine (http://www.matrixscience.com) for protein identification using default parameters [Perkins D. N., D. J. C. Pappin, D. M. Creasy and J. S. Cottrell. Probability-based protein identification by searching sequence databases using mass spectrometry data. Electrophoresis 1999; 20:3551-3567].

With the humanized SCID/SCID mice model, we were able to identify a whole array of immunogenic pneumococcal proteins after immunization with S. pneumoniae. Some of them have previously been identified as immunogenic (PhtA, PspA, PsaA, PspC, ORF SP_{—}0082, endo-β-N-acetylglucosaminidase, IgA1 protease, serine protease PrtA, α-enolase, DnaK, FBA, GAPDH, pyruvate oxidase, and phosphoglycerate kinase) in humans. PhtA, PspA, PsaA and PspC are currently being tested in clinical trials. The identification of these proteins proved the usefulness of the humanized SCID/SCID mice model and the immuno-proteomics approach. Other pneumococcal proteins identified in this study such as (zmpB, and GroEL) and protective (ABC transporter (spermidine), and ClpP) were demonstrated to be immunogenic in a human immune system. Our results, for the first time, show that these proteins are also immunogenic for the human immune system. Pneumococcal zmpC and ABC transporters (glutamine) have only been described as contributing to pneumococcal virulence in mice. Our results, however, are the first that illustrate the immunogenicity of these pneumococcal proteins after immunization with intact S. pneumoniae. In particular antibodies for two fragments of SP_{—}0082, hereinafter referred to as the SP_{—}0082 fragments SP4 and SP17, where shown to limit pneumococcal adhesion in the nasopharyngeal space. Finally, ABC transporters (maltose/maltodextrin, SP_{—}1683, SP_{—}1386), PTS system IIA component, pyruvate kinase, and hypothetical proteins (SP_{—}1290, SP_{—}0562 and SP_{—}0965) have never been identified, i.e. characterized, as immunogenic after immunization with intact S. pneumoniae, neither in mice nor in humans. Using a humanized SCID model, we were able to illustrate the immunogenicity of these proteins after immunization with intact S. pneumoniae. This protective capacity or the capacity to limit pneumococcal carriage of all these newly identified antibodies, can be used in diagnosing or treating of S. pneumoniae infections in human. It may well be possible that some of the newly identified immunogenic proteins are candidates for new protein vaccines.

The above mentioned disease related antigens are well characterized and are available for the man skilled in the art.

The S. pneumoniae zmpC, concerns the protein (see Table II) encoded by the gene zmpC (Locus names SP_{—}0071). The S. pneumoniae zmpC has also been called ZmpC metallopeptidase (MEROPS Name), Zinc metalloprotease zmpC precursor or SP0017 protein, the Uniprot accesssion number is Q97T80. The zmpC gene has been described in Tettelin H et al. Science 293:498-506(2001) and the protein in Oggioni M. R., et al. Mol. Microbiol. 49:795-805(2003). The zmpC is a zinc metalloproteinase that specifically cleaves human matrix metalloproteinase 9 (MMP-9), leading to its activation. ZmpC may play a role in pneumococcal virulence and pathogenicity in the lung. “ZmpC” has been demonstrated as virulence factor as candidate surface proteins responsible or pneumococcal infection and potentially involved in distinct stages of pneumococcal disease. Damiana Chiavolini, et al. BMC Microbiology 2003, 3 Published: 3 Jul. 2003. This article is available from: http://www.biomedcentral.com/1471-2180/3/14

TABLE II
The zmpC sequence is integrated into UniProtKB/Swiss-Prot on
01-FEB-2005 provided with accession number Q97T80
(sequence last modified on 01-OCT-2001); Entry was
last modified 24-JUL-2007 (version 32).

| | |
|---|---|
| Length | 1856 AA |
| Molecular weight | 206737 Da |

SP_{—}0562

The protein SP_{—}0562 is a putative uncharacterized protein (see Table III) of S. pneumoniae (TIGR4) that encoded by the open reading frame SP_{—}0562 (Locus names SP_{—}0562) and has been described in Tettelin H., et al. Science 293:498-506(2001).

TABLE IIIa

The sequence of SP_0562 has been integrated into UniProtKB/TrEMBL
01-OCT-2001 under the Primary accession number Q97S51 and the sequence
was last modified on 01-OCT-2001,

| | |
|---|---|
| Length | 444 AA |
| Molecular weight | 51346 Da |

| | |
|---|---|
| ----------+----------+----------+----------+----------+ | |
| MTDERIHILR DILLELHNGA SPESVQDRFD ATFTGVSAIE ISLMEHELMN | 50 |
| SDSGVTFEDV MELCDVHANL FKNAIKGVEV SDTEHPGHPV RVFKEENLAL | 100 |
| RAALIRIRRL LDTYESMEDE EMLAEMRKGL VRQMGLVGQF DIHYQRKEEL | 150 |
| FFPIMERYGH DSPPKVMWGV DDQIRELFQT ALTTAKSLPE VSISSVKEAF | 200 |
| EAFATEFESM IFKEESILLM ILLESFTQDD WLQIAEESDA YGYAIIRPSE | 250 |
| KWVPERQSFI EEKIAEEPVQ LDTAEGQVQQ VIDTPEGHFT ITFTPKEKEA | 300 |
| VLDRHSQQAF GNGYLSVEQA NLILNHLPME ITFVNKEDIF QYYNDNTPAD | 350 |
| EMIFKRTPSQ VGRNVELCHP PKYLDKVKTI MKGLREGSKD KYEMWFKSES | 400 |
| RGKFVHITYA AVHDEDGEFQ GVLEYVQDIQ PYREIDTDYF RGLE | 444 |

And SP_0562 encoded by the CDS:

| | |
|---|---|
| 1 | atgacagatg aacggattca tatcctacgg gatattttgt tagaattgca caatggcgcc |
| 61 | tctcctgagt cggttcaaga tcgctttgat gcgaccttta cgggcgtgtc agccatcgag |
| 121 | atttccctta tggagcacga gctgatgaac tcggattcgg gcgtcacttt tgaagatgtt |
| 181 | atggaactct gtgatgtcca tgccaatctt tttaaaaatg ctatcaaagg tgtcgaagtt |
| 241 | tcagatactg agcatccagg tcacccagtt cgtgtcttca aagaagaaaa tctggctctc |
| 301 | cgtgcggcct tgattcgcat tcgtagattg ttagatacct atgagtctat ggaagacgag |
| 361 | gaaatgctgg cggagatgcg taagggtttg gtgcgtcaga tgggacttgt gggtcaattt |
| 421 | gacatccatt accaacgtaa ggaagaactc ttctttccta tcatggagcg ctatggacac |
| 481 | gattcacctc ccaaagttat gtggggagtg gatgatcaga ttagggaact ctttcaaaca |
| 541 | gctctaacga cagccaagtc actaccagaa gtgtcaatta gcagtgtaaa ggaagctttt |
| 601 | gaagcttttg cgacagagtt tgaaagtatg attttcaagg aagagtccat cctcctcatg |
| 661 | attctccttg agtcttttac tcaggatgac tggcttcaga ttgcggagga gagcgatgcc |
| 721 | tatggctatg ccatcatccg tccgtcagag aaatgggtgc cagaacgaca gagctttatt |
| 781 | gaggaaaaga ttgcagagga gcctgtacag ctagatacgg cagaaggtca agttcaacaa |
| 841 | gtcatagata cgccagaagg ccattttacc attaccttta cccctaagga aaaggaagct |
| 901 | gtgctggacc gccatagtca acaggctttt ggtaatggct atctttcagt cgagcaggcc |
| 961 | aatctcatcc tcaatcatct ccctatggag attacctttg tcaataaaga agatattttc |
| 1021 | cagtattaca atgacaatac gccagctgat gagatgattt tcaaacggac gccgtcccaa |
| 1081 | gtcgggcgca atgtcgaact ctgccatccg cctaagtact tggacaaggt caaaactatc |
| 1141 | atgaaggggc ttcgtgaggg aagcaaagac aagtatgaaa tgtggttcaa gtctgagtcg |
| 1201 | cgaggtaagt ttgtccacat cacctatgct gcagtacacg atgaagacgg agaattccaa |
| 1261 | ggagtgttgg agtatgttca ggatatccag ccctaccgtg agattgatac ggactatttt |
| 1321 | cgtggattag aataa |

A particular immunogenic fragment thereof consists of the following protein and corresponding CDS (Table IIIb).

TABLE IIb

TEFESMIFKEESILLMILLESFTQDDWLQIAEESDAYGYAIIRPSEKWVPERQSFIEEKIAEEPVQLDTA
EGQVQQVIDTPEGHFTITFTPKEKEAVLDRHSQQAFGNGYLSVEQANLILNHLPMEITFVNKEDIFQYYN
DNTPADEMIFKRTPSQVGRNVELCHPPKYLDKVKTIMKGLREGSKDKYEMWFKSESRGKFVHITYAAVHD
EDGEFQGVLEYVQDIQPYREIDTDYFRGLE
encoded by the CDS
ACAGAGTTTGAAAGTATGATTTTCAAGGAAGAGTCCATCCTCCTCATGATTCTCCTTGAGTCTTTTACTC
AGGATGACTGGCTTCAGATTGCGGAGGAGAGCGATGCCTATGGCTATGCCATCATCCGTCCGTCAGAGAA
ATGGGTGCCAGAACGACAGAGCTTTATTGAGGAAAAGATTGCAGAGGAGCCTGTACAGCTAGATACGGCA
GAAGGTCAAGTTCAACAAGTCATAGATACGCCAGAAGGCCATTTTACCATTACCTTTACCCCTAAGGAAA
AGGAAGCTGTGCTGGACCGCCATAGTCAACAGGCTTTTGGTAATGGCTATCTTTCAGTCGAGCAGGCCAA
TCTCATCCTCAATCATCTCCCTATGGAGATTACCTTTGTCAATAAAGAAGATATTTTCCAGTATTACAAT
GACAATACGCCAGCTGATGAGATGATTTTCAAACGGACGCCGTCCCAAGTCGGGCGCAATGTCGAACTCT
GCCATCCGCCTAAGTACTTGGACAAGGTCAAAACTATCATGAAGGGGCTTCGTGAGGGAAGCAAAGACAA
GTATGAAATGTGGTTCAAGTCTGAGTCGCGAGGTAAGTTTGTCCACATCACCTATGCTGCAGTACACGAT
GAAGACGGAGAATTCCAAGGAGTGTTGGAGTATGTTCAGGATATCCAGCCCTACCGTGAGATTGATACGG
ACTATTTTCGTGGATTAGAATAA

SP_{—}1290

The hypotetical protein SP_{—}1290 (Table IV) has been described as a putative identification conserved hypothetical protein with a Molecular Weight 50865 and protein length 434. It is encoded by a gene of S. pneumoniae (TIGR4). The UniProtKB/TrEMBL entry name is Q97QC9_STRPN and primary accession number Q97QC9. It has been described in Tettelin H., et al. RL Science 293:498-506(2001).

TABLE IV

| | |
|---|---|
| Length | 434 AA |
| Molecular weight | 50865 Da |
| CRC64 | A99C7D3F25998A5F |

| | |
|---|---|
| ----------+----------+----------+----------+----------+ | |
| MNEKVFRDPV HNYIHVNNQI IYDLINTKEF QRLRRIKQLG TSSYTFHGGE | 50 |
| HSRFSHCLGV YEIARRITEI FEEKYPEEWN PAESLLTMTA ALLHDLGHGA | 100 |
| YSHTFEHLFD TDHEAITQEI IQNPETEIHQ VLLQVAPDFP EKVASVIDHT | 150 |
| YPNKQVVQLI SSQIDADRMD YLLRDSYFTG ASYGEFDLTR ILRVIRPIEN | 200 |
| GIAFQRNGMH AIEDYVLSRY QMYMQVYFHP ATRAMEVLLQ NLLKRAKELY | 250 |
| PEDKDFFART SPHLLPFFEK NVTLTDYLAL DDGVMNTYFQ LWMTSPDKIL | 300 |
| ADLSHRFVNR KVFKSITFSQ EDQDQLTSMR KLVEDIGFDP DYYTAIHKNF | 350 |
| DLPYDIYRPE SENPRTQIEI LQKNGELAEL SSLSPIVQSL AGSRHGDNRF | 400 |
| YFPKEMLDQN SIFASITQQF LHLIENDHFT PNKN | 434 |

ABC transporter (SP_{—}1683)

The Sugar ABC transporter, sugar-binding protein has been entered in UniProtKB/TrEMBL as Q97PE6_STRPN under the primary accession number Q97PE6. Its sequence was last modified 1 Oct. 2001 (see table V). It is encoded by the gene with the locus name SP_{—}1683. The protein has been described by Tettelin, H et al (2001) Science 293:498-506. In the immunization experiments (infra) a recombinant protein lacking the signalling sequence (underlined below) has been used.

TABLE V

| | |
|---|---|
| Length | 442 AA |
| Molecular weight | 48304 Da |

| | |
|---|---|
| ----------+----------+----------+----------+----------+ | |
| MKFRKLACTV LAGAAVLGLA ACGNSGGSKD AAKSGGDGAK TEITWWAFPV | 50 |
| FTQEKTGDGV GTYEKSIIEA FEKANPDIKV KLETIDFKSG PEKITTAIEA | 100 |
| GTAPDVLFDA PGRIIQYGKN GKLAELNDLF TDEFVKDVNN ENIVQASKAG | 150 |
| DKAYMYPISS APFYMAMNKK MLEDAGVANL VKEGWTTDDF EKVLKALKDK | 200 |
| GYTPGSLFSS GQGGDQGTRA FISNLYSGSV TDEKVSKYTT DDPKFVKGLE | 250 |
| KATSWIKDNL INNGSQFDGG ADIQNFANGQ TSYTILWAPA QNGIQAKLLE | 300 |
| ASKVEVVEVP FPSDEGKPAL EYLVNGFAVF NNKDDKKVAA SKKFIQFIAD | 350 |
| DKEWGPKDVV RTGAFPVRTS FGKLYEDKRM ETISGWTQYY SPYYNTIDGF | 400 |
| AEMRTLWFPM LQSVSNGDEK PADALKAFTE KANETIKKAM KQ | 442 |

And SP_1683 endoced by CDS:

| | |
|---|---|
| 1 | ctattgtttc atagcttttt tgattgtttc gttcgctttt tcagtgaagg ctttcaaagc |
| 61 | atctgctggt ttttcgtcac catttgatac agattgcaac attgggaacc aaagtgttct |
| 121 | catttcagca aatccatcaa tagtgttgta gtatggtgag tagtattgag tccagccgct |
| 181 | gattgtttcc atgcgtttgt cttcataaag ttttccaaat gaagtacgga ctgggaaagc |
| 241 | acctgtacga actacgtctt taggtcccca ctccttgtca tctgcgataa actggatgaa |
| 301 | tttcttagat gcagcgactt tcttgtcgtc tttattgttg aatactgcaa acccgtttac |
| 361 | aaggtactca agagctggct taccttcgtc tgatgggaat ggtacttcta ccacttctac |
| 421 | cttacttgct tctaaaagtt tagcttggat accattttga gctggtgccc aaaggattgt |
| 481 | gtaagatgtt tgaccgttgg caaagttttg gatatctgcc ccaccgtcaa attgtgaacc |
| 541 | attattgatc aaattgtctt taatccagct agttgctttt tcaagacctt tgacgaattt |
| 601 | aggatcatca gttgtatatt tgctaacttt ttcatctgtt acagaaccgc tataaaggtt |
| 661 | agagataaag gcacgtgttc cttggtctcc cccttgacca gaactgaaca atgaacctgg |
| 721 | tgtgtaaccc ttgtctttaa gtgctttcaa tactttttca aaatcatcag ttgtccaacc |
| 781 | ttcttttaca aggtttgcta ctccagcatc ttctaacatt ttcttgttca ttgccatgta |
| 841 | gaatggggca gaactaatcg gatacatata agccttgtct ccagctttac ttgcttgtac |
| 901 | gatgttttca ttgttgacat ctttaacaaa ttcatctgtg aagaggtcat tcaactcagc |
| 961 | caatttaccg tttttaccgt attggatgat acgtcctggt gcatcaaaga gtacgtctgg |
| 1021 | agctgttcct gcttcgatgg ctgttgtgat tttttcagga cctgacttga agtcgatggt |
| 1081 | ttccaatttc acttttatat ctgggtttgc tttttcaaac gcttcgatga ttgatttttc |
| 1141 | ataagttcca acaccgtcac cagttttttc ttgggtaaat actgggaatg cccaccaagt |
| 1201 | gatttctgtt ttggcaccgt caccacctga tttggcagca tctttacttc cgccagaatt |
| 1261 | gccacaagca gcaagaccaa gaaccgcagc acccgcaagt actgtacaag ctaattttct |
| 1321 | aaatttcat |

ABC Transporter (Glutamine)

Streptococcus pneumoniae R6 ABC transporter ATP-binding protein-glutamine transport UniProtKB/TrEMBL as Q8DP49_STRR6 under the primary accession number Q8D49 [10.1128/JB.183.19.5709-5717.2001; PubMed=11544234 [NCBI, ExPASy, EBI, Israel, Japan]Hoskins J., Alborn W. E. Jr., Arnold J., Blaszczak L. C., Burgett S., DeHoff B. S., Estrem S. T., Fritz L., Fu D.-J., Fuller W., Geringer C., Gilmour R., Glass J. S., Khoja H., Kraft A. R., Lagace R. E., LeBlanc D. J., Lee L. N., Lefkowitz E. J., Glass J. I.; “Genome of the bacterium Streptococcus pneumoniae strain R6.”; J. Bacteriol. 183:5709-5717(2001)]. see table VI Moleculair Weight: 23144 Da and length: 209AA

The CDS is

TABLE VI

atgttagaat tacgaaatat caataaagtc tttggagaca aacaaatcct gtctaatttc agtctaagta ttcctgaaaa gcaaatcctg
gctatcgttg gaccttctgg tggaggtaag acaactcttt tacgtatgct tgcaggtctt gaaaccattg attcagggca aatcttttat
aatggacaac ctttagagct ggatgaattg cagaagcgca atctactggg atttgtcttc caagattttc aactatttcc tcatctatca
gttctggaaa atttgacttt atcgcctgtg aagaccatgg gaatgaagca ggaagaggct gagaagaagg cgagtggact
cttggaacag ttaggactag gaggacacgc agaggcctat cctttctcac tatctggtgg gcaaaagcag cgggtggctt
tggcgcgtgc tatgatgatt gacccagaaa tcattggcta cgatgaacca acttctgccc tggatccaga attacgtttg
gaagtggaga agctaatctt gcaaaatagg gaacttggga tgacccagat tgtggttacc catgatttgc agtttgctga
aaatatcgca gatgtattat tgaaagtaga acctaaatag
which can be translated in
MLELRNINKVFGDKQILSNFSLSIPEKQILAIVGPSGGGKTTLLRMLAGLETIDSGQIFYNG
QPLELDELQKRNLLGFVFQDFQLFPHLSVLENLTLSPVKTMGFTMKQEEAEKKASGLLE
QLGLGGHAEAYPFSLSGGQKQRVALARAMMIDPEIIGYDEPTSAFTLDPELRLEVEKLIL
QNRELGMTQIVVTHDLQFAENIADVLLKVEPK”

ABC Transporter (Maltose/Maltodextrin)

A sugar ABC transporter, sugar-binding protein of S. pneumoniae (TIGR4) has also been entered in UniProtKB/TrEMBL as Q04I66_STRP2 under the primary accession number Q04I66 (Locus name: SPD_{—}1934). 10.1128/JB.01148-06; PubMed=17041037 [NCBI, ExPASy, EBI, Israel, Japan]Lanie J. A., Ng W.-L., Kazmierczak K. M., Andrzejewski T. M., Davidsen T. M., Wayne K. J., Tettelin H., Glass J. I., Winkler M. E.; “Genome sequence of Avery's virulent serotype 2 strain D39 of Streptococcus pneumoniae and comparison with that of unencapsulated laboratory strain R6.”; J. Bacteriol. 189:38-51(2007). see table VII Moleculair Weight: 45367 Da and length: 423AA

TABLE VII
The CDS is
atgtcatcta aatttatgaa gagcactgcg gtgcttggaa ctgttacact tgctagcttg cttttggtag cttgcggaag caaaactgct
gataagcctg ctgattctgg ttcatctgaa gtcaaagaac tcactgtata tgtagacgag ggatataaga gctatattga agaggttgct
aaagcttatg aaaaagaagc tggagtaaaa gtcactctta aaactggtga tgctctagga ggtcttgata aactttctct tgacaaccaa
tctggtaatg tccctgatgt tatgatggct ccatacgacc gtgtaggtag ccttggttct gacggacaac tttcagaagt gaaattgagc
gatggtgcta aaacagacga cacaactaaa tctcttgtaa cagctgctaa tggtaaagtt tacggtgctc ctgccgttat cgagtcactt
gttatgtact acaacaaaga cttggtgaaa gatgctccaa aaacatttgc tgacttggaa aaccttgcta aagatagcaa
atacgcattc gctggtgaag atggtaaaac tactgccttc ctagctgact ggacaaactt ctactataca tatggacttc ttgccggtaa
cggtgcttac gtctttggcc aaaacggtaa agacgctaaa gacatcggtc ttgcaaacga cggttctatc gcaggtatca
actacgctaa atcttggtac gaaaaatggc ctaaaggtat gcaagataca gaaggtgctg gaaacttaat ccaaactcaa
ttccaagaag gtaaaacagc tgctatcatc gacggacctt ggaaagctca agcctttaaa gatgctaaag taaactacgg
agttgcaact atcccaactc ttccaaatgg aaaagaatat gctgcattcg gtggtggtaa agcttgggtc attcctcaag
ccgttaagaa ccttgaagct tctcaaaaat ttgtagactt ccttgttgca actgaacaac aaaaagtatt atatgataag actaacgaaa
tcccagctaa tactgaggct cgttcatacg ctgaaggtaa aaacgatgag ttgacaacag ctgttatcaa acagttcaag
aacactcaac cactgccaaa catctctcaa atgtctgcag tttgggatcc agcgaaaaat atgctctttg atgctgtaag
tggtcaaaaa gatgctaaaa cagctgctaa cgatgctgta acattgatca aagaaacaat caaacaaaaa tttggtgaat aa
which can be translated in:
MSSKFMKSTAVLGTVTLASLLLVACGSKTADKPADSGSSEVKELTFTVYVDEGYK
SYIEEVAKAYEKEAGVKVTLKTGDALGGLDKLSLDNQSGNVPDVMMAPYDRFTV
GSLGSDGQLSEVKLSDGAKTDDTTKSLVTAANGKVYGAPAVIESLVMYYNKDLVK
DAPFTKTFADLENLAKDSKYAFAGEDGKTTAFLADWTNFYYTYGLLAGNGAYVFG
QNGKDAKDIFTGLANDGSIAGINYAKSWYEKWPKGMQDTEGAGNLIQTQFQEGKTA
AIIDGPWKAQAFKDFTAKVNYGVATIPTLPNGKEYAAFGGGKAWVIPQAVKNLEASQ
KFVDFLVATEQQKVLYDKFTTNEIPANTEARSYAEGKNDELTTAVIKQFKNTQPLPNIS
QMSAVWDPAKNMLFDAVSGQFTKDAKTAANDAVTLIKETIKQKFGE

PTS System IIA Component (Mannose)

The bacterial phosphoenolpyruvate: sugar phosphotransferase system (PTS) (see Table VIIIa & VIIIb) is a multi-protein system involved in the regulation of a variety of metabolic and transcriptional processes. PEP group translocation, also known as the phosphotransferase system or PTS, is a distinct method used by bacteria for sugar uptake where the source of energy is from phosphoenolpyruvate. It is known as multicomponent system that always involves enzymes of the plasma membrane and those in the cytoplasm. Sugar Phosphotransferase System (PTS) is considered a target for antibacterials. The bacterial phosphoenolpyruvate: sugar phosphotransferase system (PTS) mediates the uptake and phosphorylation of carbohydrates, and is involved in signal transduction. Enzyme I (EI) is the first component of the PTS cascade, conserved and ubiquitous in bacteria but absent in animals and plants. EI has been implicated in virulence (Mukhija S. et al Abstr Intersci Conf Antimicrob Agents Chemother Intersci Conf Antimicrob Agents Chemother. 2002 Sep. 27-30; 42: ARPIDA, Munchenstein, Switzerland. Tettelin, H., et al. Science 293 (5529), 498-506(2001) described the PTS system, IIA component of S. pneumoniae TIGR4 which has been deposited in the NCBI database under the accession number NP_{—}346461 (submitted on 3 Oct. 2001). PTS system, IIA component 1.161 is part of the region, PTS_IIA_fru. The PTS system, fructose/mannitol specific IIA subunit.

TABLE VIIIa
The PTS system IIA component (mannose) protein
sequence is:

| | |
|---|---|
| 1 | mnlkqalidn dsirlglean nwkeavkvav dpliesgail |
| | peyydaiies teeygpyyil |
| 61 | mpgmamphar peagvqsdaf slitlqnpvv fsdgkevsvl |
| | lalaatsski htsvaipqii |
| 121 | alfeledsia rlqacqtked vlamieeskd spylegldle s |

A PTS system, IIA component for the strain S. pneumoniae D39] has been deposited in the NCBI database under accession number YP_{—}817378 and the DBsource Refseq: accession NC_{—}008533.1 (Table VIII (b)) It has been described by Lanie, J. A., et al. J. Bacteriol. 189 (1), 38-51 (2007).

TABLE VIIIb

The PTS system IIA component (mannose) CDS:

| | |
|---|---|
| 1 | ctatagacat aaatcctctt cctcctctac gttaagttgt tgtttaacat taaacaaact |
| 61 | attttgagca ttgtgaacaa tttcttctaa attgattttt gaatcataac ttgatattaa |
| 121 | ttccactagg agagggatat taaaccctgt tacaatatca actgaatcca aatttaaaaa |
| 181 | ccgtgacaaa gccacattat taggacttcc tccaatcaag tcagtcaaaa cgataacctc |
| 241 | ttgatttgag tctaacagtt catttacttg atttttaaaa taatgttcaa actctacaat |
| 301 | attctctcca ggcatcaaac ctaatgtcct aactctatca tttgttgtct caccaacaaa |
| 361 | catttctgcg gtcatgagag ctccgctagc aaaattacca tgactggcaa ctaaaattcc |
| 421 | tctattaaac attttctctt tcaa |

which can be translated in the protein sequence:

| | |
|---|---|
| 1 | mkekmfnrgi lvashgnfas galmtaemfv gettndrvrt lglmpgeniv efehyfknqv |
| 61 | nelldsnqev ivltdliggs pnnvalsrfl nldsvdivtg fnipllveli ssydskinle |
| 121 | eivhnaqnsl fnvkqqlnve eeedlcl |

Pyruvate Kinase

Pyruvate kinase has been described for various S. pneumoniae strains. Tettelin, H et al. described this for Science 293 (5529), 498-506 (2001). Pyruvate kinase of S. pneumoniae TIGR4 has for instance been deposited in the NVBI database under accesion number NP_{—}345384. Pyruvate kinase (PK) is a large allosteric enzyme that regulates glycolysis through binding of the substrate, phosphoenolpyruvate, and one or more allosteric effectors. Endphase in the glykolyse scheme enolase regulates the transformation of Triphospoglycerate into phosphoenolpyruvate and pyruvate kinase regulates phosphoenolpyruvate transformation into pyruvate. Like other allosteric enzymes, PK has a high substrate affinity R state and a low affinity T state. PK exists as several different isozymes, depending on organism and tissue type. In mammals, there are four PK isozymes: R, found in red blood cells, L, found in liver, M1, found in skeletal muscle, and M2, found in kidney, adipose tissue, and lung. PK forms a homotetramer, with each subunit containing three domains. The T state to R state transition of PK is more complex than in most allosteric enzymes, involving a concerted rotation of all 3 domains of each monomer in the homotetramer.

GeneID:930850 encodes the pyruvate kinase with Sequence NP_{—}345384 (see table IX)

TABLE IX

The pyruvate kinase AA sequence

| | |
|---|---|
| 1 | mnkrvkivat lgpaveirgg kkfgedgywg ekldveasak niaklieaga ntfrfnfshg |
| 61 | dhqeqgerma tvklaekiag kkvgflldtk gpeirtelfe geakeysykt gekirvatkq |
| 121 | gikstrevia lnvagaldiy ddvevgrqvl vddgklglrv vakddatref evevendgii |
| 181 | akqkgvnipn tkipfpalae rdnddirfgl eqginfiais fvrtakdvne vraiceetgn |
| 241 | ghvqlfakie nqqgidnlde iieaadgimi argdmgievp femvpvyqkm iikkvnaagk |
| 301 | vvitatnmle tmtekpratr sevsdvfnav idgtdatmls gesangkypl esvttmatid |
| 361 | knaqallney grldsdsfer nsktevmasa vkdatssmdi klvvtltktg htarliskyr |
| 421 | pnadilaltf delterglml nwgvipmltd apsstddmfe iaerkaveag lvesgddivi |
| 481 | vagvpvgeav rtntmrirtv r |

Other deposits are YP_{—}816275 for pyruvate kinase [S. pneumoniae D39]; ABJ55166 for pyruvate kinase [S. pneumoniae D39]; NP_{—}345384 for pyruvate kinase [S. pneumoniae TIGR4]; AAK75024 for pyruvate kinase [S. pneumoniae TIGR4]; ZP_{—}01834800 for pyruvate kinase [S. pneumoniae SP23-BS72]; ZP_{—}01831836 for pyruvate kinase [S. pneumoniae SP19-BS75]; ZP_{—}01830820 for pyruvate kinase [S. pneumoniae SP18-BS74]; ZP01827374 for pyruvate kinase [S. pneumoniae SP14-BS69]; ZP_{—}01824671 for pyruvate kinase [S. pneumoniae SP11-BS70]; ZP_{—}01822594 for pyruvate kinase [S. pneumoniae SP9-BS68]; ZP_{—}01819773 for pyruvate kinase [S. pneumoniae SP6-BS73]; ZP 01817901 for pyruvate kinase [S. pneumoniae SP3-BS71]; EDK81836 for pyruvate kinase [S. pneumoniae SP23-BS72]; EDK79443 for pyruvate kinase [S. pneumoniae SP9-BS68]; EDK77081 for pyruvate kinase [S. pneumoniae SP6-BS73]; EDK74139 for pyruvate kinase [S. pneumoniae SP3-BS71]; EDK71875 for pyruvate kinase [S. pneumoniae SP19-BS75]; EDK68161 for pyruvate kinase [S. pneumoniae SP18-BS74] and EDK66369 for pyruvate kinase [S. pneumoniae SP14-BS69]; EDK63757 for pyruvate kinase [S. pneumoniae SP11-BS70]. At least these sequences as published before or on the filing date of this application are thereby incorporated by reference.

zmpB:

ZmpB or zinc metalloprotease ZmpB (see Table X(a), Table X (b) & Table X (c)) is know to induce tumor necrosis factor alpha production in the respiratory tract and to a virulent factor of S. pneumoniae. Infection and Immunity, September 2003, p. 4925-4935, Vol. 71, No. 9. Tettelin, H., et described ZmpB in Science 293 (5529), 498-506 (2001). It has been deposited in the NCBI database under the accession number AAK74809.

TABLE X (a)

The zmpB AA sequence:

| | |
|---|---|
| 1 | mapsvvdaat yhyvnkeiis qeakdliqtg kpdrnevvyg lvyqkdqlpq tgteasvlta |
| 61 | fglltvgsll liykrkkias vflvgamglv vlpsagavdp vatlalasre gvvemegyry |
| 121 | vgylsgdilk tlgldtvlee tsakpgevtv vevetpqsit nqeqartenq vveteeapke |
| 181 | eapkteespk eepksevkpt ddtlpkveeg kedsaepapv eevggevesk peekvavkpe |
| 241 | sqpsdkpaee skveqagepv apredekapv epekqpeape eekaveetpk qeestpdtka |
| 301 | eetvepkeet vnqsieqpkv etpavekqte pteepkveqa gepvaprede qaptapvepe |
| 361 | kqpevpeeek aveetpkped kikgigtkep vdkselnnqi dkassvsptd ystasynalg |
| 421 | pvletakgvy asepvkqpev nsetnklkta idalnvdkte lnntiadakt kvkehysdrs |
| 481 | wqnlqtevtk aekvaantda kqsevneave kltatieklv elsekpiltl tstdkkiler |
| 541 | eavakytlen qnktkiksit aelkkgeevi ntvvltddkv ttetisaafk nleyykeytl |
| 601 | sttmiydrgn geetetlenq niqldlkkve lknikrtdli kyengketne slittipddk |
| 661 | snyylkitsn nqkttllavk nieettvngt pvykvtaiad nlvsrtadnk feeeyvhyie |
| 721 | kpkvhednvy ynfkelveai qndpskeyrl gqsmsarnvv pngksyitke ftgkllsseg |
| 781 | kqfaiteleh plfnvitnat innvnfenve iersgqdnia slantmkgss vitnvkitgt |
| 841 | lsgrnnvagf vnnmndgtri envaffgklh stsgngshtg giagtnyrgi vrkayvdati |
| 901 | tgnktrasll vpkvdygltl dhligtkall tesvvkgkid vsnpvevgai asktwpvgtv |
| 961 | snsvsyakii rgeelfgsnd vddsdyasah ikdlyavegy ssgnrsfrks ktftkltkeq |
| 1021 | adakvttfni tadklesdls plaklneeka yssiqdynae ynqayknlek lipfynkdyi |
| 1081 | vyqgnklnke hhlntkevls vtamnnnefi tnldeankii vhyadgtkdy fnlssssegl |
| 1141 | snvkeytitd lgikytpniv qkdnttlvnd iksilesvel qsqtmyqhln rlgdyrvnai |
| 1201 | kdlyleesft dvkenltnli tklvqneehq lndspaarqm irdkveknka alllgltyln |
| 1261 | ryygvkfgdv nikelmlfkp dfygekvsvl drlieigske nnikgsrtfd afgqvlakyt |
| 1321 | ksgnldafln ynrqlftnid nmndwfidat edhvyiaera seveeiknsk hrafdnlkrs |
| 1381 | hlrntilpll nidkahlyli snynaiafgs aerlgkksle dikdivnkaa dgyrnyydfw |
| 1441 | yrlasdnvkq rllrdavipi wegynapggw vekygryntd kvytplreff gpmdkyynyn |
| 1501 | gtgayaaiyp nsddirtdvk yvhlemvgey gisvythett hvndraiylg gfghregtda |
| 1561 | eayaqgmlqt pvtgsgfdef gslginmvfk rkndgnqwyi tdpktlktre dinrymkgyn |
| 1621 | dtltlldeie aesvisqqnk dlnsawfkki dreyrdnnkl nqwdkirnls qeeknelniq |
| 1681 | svndlvdqql mtnrnpgngi ykpeaisynd qspyvgvrmm tgiyggntsk gapgavsfkh |
| 1741 | nafrlwgyyg yengflgyas nkykqqsktd gesvlsdeyi ikkisnntfn tieefkkayf |
| 1801 | kevkdkatkg lttfevngss vssyddlltl fkeavkkdae tlkqeangnk tvsmnntvkl |
| 1861 | keavykkllq qtnsfktsif k |

The zinc metalloprotease ZmpB [S. pneumoniae SP23-BS72] has been deposited in NCBI under the accession number ZP_{—}01835170 or the dbsource refseq accession NZ_ABAG01000005.1 with a protein sequence as in table X(b)

TABLE X (b)

The AmpB AA sequence:

| | |
|---|---|
| 1 | mfkkdrfsir kikgvvgsvf lgsllmapsv vdaatyhyvn keiisqeakd liqtgkpdrn |
| 61 | evvyglvyqk dqlpqtgtea svltafgllt vgsllliykr kkiasvflvg amglvvlpsa |
| 121 | eavdpvatla lasregvvem dgyryvgyls gdilktlgld tvleetsaqp gevtvvevet |
| 181 | pqsttnqeqa rtenqvvete eapkeeapkt eespkeepks eikptddtlp kveegkedsa |
| 241 | epapveevgg eveskpeekv avkpesqpsd ksaeeskvep pveqakvpeq pvqptqaeqp |
| 301 | stpkessqed nskedrgaee tpkqedeqpa eapeikveep veskeetvnq pveqpkvetp |
| 361 | avekqtepte epkvevttve treevipfet keqeddtlkr gtrqvvqkgv egkkqitety |
| 421 | ktirgektne apivketvie qpqdeiikkg tkglekptlq wantekdvlk ksatasytlt |
| 481 | kpagveiksi klalkdkdgq lvkevtvaen nlnatldklk yyqgytlstt mvydrgegee |
| 541 | tekledkqiq ldlkkveikn iketslmnvd aegnetdksl lsekptdvsq lylrvtthdn |
| 601 | kvtrlavssv eevvvdgktl ykvvakapdl vqrraddtls eeyvhyfekq lpkvnnvyyn |
| 661 | fnelvkdmqa nptgefklga dlnaanvkpn gksyvtkpfk gkllsndggr ftihnierpl |
| 721 | fanieggkih dinlanvnin mpwadkvapi anviknnati envkvtgnvl gkdwvsgfid |
| 781 | kidgsgklin vafignvtsv gtggnfltgi vgenwkgyve rayvdahikg krakaagiay |
| 841 | wsqnqgnnft igsegaikks vvkgtidvek pievggavgs ftyhgsiedt vsmmkvknge |
| 901 | ifygskdidd dpyytgnhvn rnyvvigvse gtstyrysnq hnrikpitqs eadvkiaeta |
| 961 | itadkftitd pivnklnalt trdneyrttq dyeatreqay rnieklqpfy nkewivnqgn |
| 1021 | klvegsnllt kevlsvtgik sgqfvtdlsd idkimihysd gakeelnvtr qesnvqqvre |
| 1081 | ysitdldily tpnmvekdra qlmtdvkskl ssvelesdgv rqllvkrdtk kdanansvgr |
| 1141 | qngyirdlfl eesfsevkan ldklvkqile nedhqlndne laerallkkv ednkakimmg |
| 1201 | laylnqyyaf kydelsikdi mmfkpdfygk tasvidrlin igsaennlkg drtqdayrgi |
| 1261 | isgatgkgsl hdfltynmkl ftnetdinvw fkkaieknay vveqpstnpa fankkyrlye |
| 1321 | ginngqhgrm ilpllnlkna hlfmistynt isfssfekyg kdtdekrekf kseinkrake |
| 1381 | qvnyldfwsr latdnvrdkl lksqnvvptp vwdnhnspng wasrhghidg kpdyapiref |
| 1441 | fgrinkyhgy kygygayayi faapqpmdav yfvmtdlisd fgtsafthet thvndrmayy |
| 1501 | gghwhregtd leafaqgmlq tpsvsnpnge ygalglnmay erqndgnqwy ntnpndlttr |
| 1561 | aeidnymkgf ndtlmlldyl egeavlnkan qdlnnawfkk vdkqlrgast knqydkvrdl |
| 1621 | tadeknitln svddlvdnnf mtkhgpgnnv ydptgfgtay vtvpitagiy ggntsegapg |
| 1681 | smsfkhntfr mwgyygyekg flnyasnmlk nesrqaghnt lgddfiikkv sdnkfstled |
| 1741 | wkkayfkevv dkakagfnpv tidsttyssy ddlknafaaa vekdkatlkn gsvksdntva |
| 1801 | lkekiykkll qqtdsfktsi fk |

Yet another deposit was zinc metalloprotease ZmpB [S. pneumoniae SP14-BS69]. with the accession number ZP_{—}01829019 and the dbsource refseq accession NZ_ABAD01000025.1. The CDS sequence and encoded protein is described in Table X (c)

TABLE X (c)

The ZmpB CDS sequence:

| | |
|---|---|
| 1 | tcataattta agttggcaga acgattcata gcatcttctg ttactttggt caaggttaaa |
| 61 | acaggagtcc gttttttctt ttggtctttc ttttgtacct tttacaataa tttcaggctt |
| 121 | catttctttc aaaattgtta ctgtttcaac tgactcgctg atttttttgc catgtaactc |
| 181 | ttcatagcta gtaacaattt gacgttcgcc atcctcacca acttgtttaa cagaattatc |
| 241 | gcctataaat ttgctaggat cagattcttc tatagtggtt ttaggaatag tctctgtacg |
| 301 | aatatcggta tgtaaagaag gcaactcttc tctttctgat gcaacttctg aaactgtaga |
| 361 | tattggttca gtatactctg gaatatcgtt aacaggtggc tcaatcaagt ttccattttc |
| 421 | atctactcct gttgtaccta caggttcagt gtatgctagt ttctcatgaa cttcaggctc |
| 481 | aacaaggttt gcacctattg gttgcgtata gtcaggcttc tcatgaactt ctggttcgcc |
| 541 | tttttcagtt acaactgctt cgggtaaggc tggttgtact tctggttccc ctttttcagc |
| 601 | cacaacagct tctggtaaca ctggttgtac ttccggttcc cctttttcag ccacaacagc |
| 661 | ttctggtaac actggttgta cttccggttc ccctttttca gtcacaacag cttctggtaa |
| 721 | tgtcggttgt acttcagttt tcccttttcc agttacaaca gcttctggta acgctggctg |
| 781 | aacttctggt tcgcctttgt cggtcacaac tttgggttgc tcaactggag aaacagttct |
| 841 | atcatcaagc tccaagtaat caacatatct gtaaccagaa atttgcaaca caccatcacg |
| 901 | tccagactca tggatgttag cattcaaatt aagtgctgaa gcagttgata gtgtaactaa |
| 961 | acctgtcgcc cctacaatca aaaatgtagc gatttttttc ctctttttat cttttgtgat |
| 1021 | aatcacaata agactcccga tagctaacaa acccaatgca gtcataatag attgagaact |
| 1081 | tcctgtgttt ggtaatgcgt ctttttcata aaccaaagca tatgactctt ttgattcatc |
| 1141 | aggtctacct tgtttgagtt gaacacgttc agtttgtgtc aaagtactat aatctaagta |
| 1201 | atgataagtc gatgtaccaa caactgacgg tgcaaataaa agactcccca agaataccga |
| 1261 | accaacaata ccctttattt tccgaatgga aaatttatct ttttttaata atgacaa |

Which is translated in the ZmpB protein fragment:

| | |
|---|---|
| 1 | msllkkdkfs irkikgivgs vflgsllfap svvgtstyhy ldystltqte rvqlkqgrpd |
| 61 | eskesyalvy ekdalpntgs sqsimtalgl laigslivii tkdkkrkkia tflivgatgl |
| 121 | vtlstasaln lnanihesgr dgvlqisgyr yvdylelddr tvspveqpkv vtdkgepevq |
| 181 | palpeavvtg kgktevqptl peavvtekge pevqpvlpea vvaekgepev qpvlpeavva |
| 241 | ekgepevqpa lpeavvtekg epevhekpdy tqpiganlve pevheklayt epvgttgvde |
| 301 | ngnlieppvn dipeytepis tvsevasere elpslhtdir tetipkttie esdpskfigd |
| 361 | nsvkqvgedg erqivtsyee lhgkkisesv etvtilkemk peiivkgtke rpkektdscf |
| 421 | nldqsnrrcy esfcqlkl |

Other accessions are for instance 5:YP_{—}816076 zinc metalloprotease ZmpB [S. pneumoniae D39] and ABJ54076 zinc metalloprotease ZmpB [S. pneumoniae D39]; AAK74809 zinc metalloprotease ZmpB [S. pneumoniae TIGR4].

ABC Transporter (Spermidine)

ABC transporter concerns a Peptide Inducible Signal Transduction System in S. pneumoniae. It has been deposited under embl accession AJ278419.1 and in the NVBI database under accession number AJ278419. Spermidine/putrescine ABC transporter, periplasmic spermidine/putrescine-binding protein and spermidine/putrescine ABC transporter, ATP-binding protein are described by Polissi, A. et al (1998). Infect Immun 66, 5620-5629. It is most likely part of a binding-protein-dependent transport system, probably responsible for the translocation of the substrate across the membrane.

Identified sequence residues are described in table XI and in other database deposits

TABLE XI

Translated ABC transporter protein:

| | |
|---|---|
| 1 | mkstlgiisv glvityilqq vmsfsrdyll tvlsqrlsid vilsyirhif elpmsffatr |
| 61 | rtgeiisrft dansiidala stilslfldv sililvggvl laqnpnlfll slisipiymf |
| 121 | iifsfmkpfe kmnhdvmqsn smvssaiied ingietiksl tseenryqni dsefvdylek |
| 181 | sfklskysil qtslkqgtkl vlnililwfg aqlvmsskis igqlitfntl fsyfttpmen |
| 241 | iinlqtklqs akvannrlne vylvesefqv qenpvhshfl mgdiefddls ykygfg |

endoced by the CDS:

| | |
|---|---|
| 1 | tcatccaaaa ccatacttat aagaaaggtc atcaaattca atatcgccca tcaaaaaatg |
| 61 | tgaatgaaca gggttttctt gaacttgaaa ttcagattcg actagataga cttcgttcaa |
| 121 | acggttatta gcgaccttcg cagattggag tttggtttgg aggttgataa tattttccat |
| 181 | aggagttgta aagtaagaaa aaagtgtgtt aaaggtaatc agctgaccga tagaaatttt |
| 241 | acttgacatg actaattgag cgccaaacca taggataagg atattcagaa ctaattttgt |
| 301 | tccctgcttt aaactcgttt gtaaaataga atatttactg agcttaaagg atttttccaa |
| 361 | ataatctaca aattcgctgt ctatattttg atagcgattt tcttcactcg tgagcgactt |
| 421 | tatagtttca atcccgttga tatcttcgat aatggcagag ctaaccatag aattactttg |
| 481 | catgacatca tggttcattt tttcgaaagg tttcataaaa gaaaagatga tgaacatgta |
| 541 | tataggaatg gaaataagag aaagaaggaa gagattaggg ttttgtgcca gtaagacgcc |
| 601 | tcctacaaga atcagaatag aaacatccag aaaaagagaa agaatggtag aagccaaggc |
| 661 | atctataata gagttagcat ctgtgaatcg tgaaatgatt tctcctgtac gacgtgtcgc |
| 721 | aaagaaagac atgggaagtt caaaaatatg gcgaatatag gataaaatca catcaatact |
| 781 | taatctctga ctcagaacgg ttaggagata atctctggag aagctcatga cttgttggag |
| 841 | gatataggtg ataaccagac caactgagat gattcctaaa gttgatttca t |

Other NCBI deposits are CAC18584 ABC transporter [S. pneumoniae]; CAC18586 DABC transporter [S. pneumoniae]; CAC18583 ABC transporter [S. pneumoniae]; AAG12999 ABC-transporter [S. pneumoniae]; NP_{—}625058 ABC transporter [Streptomyces coelicolor A3(2)]; AAL73130 ABC-transporter [S. pyogenes]; ABC70432 putative ABC transporter [S. pneumoniae]; CAC18604 ABC transporter ATP binding domain [S. pneumoniae]; CAC18603 putative ABC transporter transmembrane domain [S. pneumoniae]; CAC03519 putative ABC transporter B1pAORF1 [S. pneumoniae]; P35598 Putative ABC transporter ATP-binding protein exp8 (Exported protein 8); Q97SA3

Reports, Putative ABC transporter ATP-binding protein SP_{—}0483; P0A4G2 Manganese ABC transporter substrate-binding lipoprotein precursor (Pneumococcal surface adhesin A); P0A2V8 Phosphate import ATP-binding protein pstB 3 (Phosphate-transporting ATPase 3) (ABC phosphate transporter 3); P63373 Phosphate import ATP-binding protein pstB 1 (Phosphate-transporting ATPase 1) (ABC phosphate transporter 1); Q97Q34 Phosphate import ATP-binding protein pstB 2 (Phosphate-transporting ATPase 2) (ABC phosphate transporter 2); NP_{—}359270 ABC transporter ATP-binding protein—choline transporter [S. pneumoniae R6]; NP_{—}359269 ABC transporter membrane-spanning permease—choline transporter [S. pneumoniae R6] and AAL00481 ABC transporter ATP-binding protein—choline transporter [S. pneumoniae R6]. At least these sequences as published before or on the filing date of this application are thereby incorporated by reference.

GroEL

The GroEL protein of S. pneumoniae (Table XII) has been deposited in the NCBI database under the accession number AAL55997 (01-DEC-2000)) and the other database deposits described hereunder.

TABLE XII

The GroEL AA sequence:

| | |
|---|---|
| 1 | mskeikfssd arsamvrgvd iladtvkvtl gpkgrnvvle ksfgsplitn dgvtiakeie |
| 61 | ledhfenmga klvsevaskt ndiagdgttt atvltqaivr egiknvtaga npigirrgie |
| 121 | tavaaaveal knnaipvank eaiaqvaavs srsekvgeyi seamekvgkd gvitieesrg |
| 181 | metelevveg mqfdrgylsq ymvtdsekmv adlenpyili tdkkisniqe ilpllesilq |
| 241 | snrplliiad dvdgealptl vlnkirgtfn vvavkapgfg drrkamledi ailtggtvit |
| 301 | edlglelkda tiealgqaar vtvdkdstvi vegagnpeai shrvaviksq ietttsefdr |
| 361 | eklqerlakl sggvavikvg aatetelkem klriedalna traaveegiv agggtalanv |
| 421 | ipavatlelt gdeatgrniv lraleepvrq iahnagfegs ividrlknae lgigfnaatg |
| 481 | ewvnmidqgi idpvkvsrsa lqnaasvasl iltteavvan kpepvapapa mdpsmmggmm |

Other deposits of GroEL are for instance AAD23455 chaperonin GroEL [S. pneumoniae]; P0A335 60 kDa chaperonin (Protein Cpn60) (groEL protein)

gi|61220901|sp|P0A335|CH60_STRPN[61220901]; ZP_{—}01831030 chaperonin GroEL [S. pneumoniae SP18-BS74]; ZP_{—}0829178 chaperonin GroEL [S. pneumoniae SP14-BS69]; ZP_{—}01825884 chaperonin GroEL [S. pneumoniae; SP11-BS70]; ZP_{—}01818677 chaperonin GroEL [S. pneumoniae SP3-BS71]; EDK73329 chaperonin GroEL [S. pneumoniae SP3-BS71]; EDK67928 chaperonin GroEL [S. pneumoniae SP18-BS74]; EDK64652 chaperonin GroEL [S. pneumoniae SP14-BS69]; EDK62682 chaperonin GroEL [S. pneumoniae SP11-BS70]; NP_{—}346336 chaperonin GroEL [S. pneumoniae TIGR4]; NP_{—}359314 chaperonin GroEL [S. pneumoniae R6]; AAL00525 Chaperonin GroEL [S. pneumoniae R6]; ZP_{—}01821355 chaperonin GroEL [S. pneumoniae SP6-BS73]; ZP_{—}01821354; chaperonin GroEL [S. pneumoniae SP6-BS73]; EDK75597 chaperonin GroEL [S. pneumoniae SP6-BS73]; EDK75596 chaperonin GroEL [S. pneumoniae SP6-BS73]; ZP_{—}01836040 chaperonin GroEL [S. pneumoniae SP23-BS72] and ZP_{—}01833546 chaperonin GroEL [S. pneumoniae SP19-BS75]. At least these sequences as published before or on the filing date of this application are thereby incoporated by reference.

The ABC Transporter (Spermidine) SP_{—}1386

The ABC transporter (spermidine) SP_{—}1386 is a member of the PBPb Super-family: use membrane-bound complexes and substrate-bound, membrane-associated, periplasmic binding proteins (PBPs) to transport a wide variety of substrates, such as, amino acids, peptides, sugars, vitamins and inorganic ions. PBPs have two cell membrane translocation functions: bind substrate, and interact with the membrane bound complex. The SP_{—}1386 protein as used herein has the amino acid sequence as shown in Table XIII below. The recombinant protein used in the immunization experiments (infra) lacked the underlined fragment.

TABLE XIII

MKKIYSFLAGIAAIILVLWGIATHLDSKINSRDSQKLVIYNWGDYIDPELLTQFTEETGIQVQYETFDSN
EAMYTKIKQGGTTYDIAIPSEYMINKMKDEDLLVPLDYSKIEGIENIGPEFLNQSFDPGNKFSIPYFWGT
LGIVYNETMVDEAPEHWDDLWKPEYKNSIMLFDGAREVLGLGLNSLGYSLNSKDLQQLEETVDKLYKLTP
NIKAIVADEMKGYMIQNNVAIGVTFSGEASQMLEKNENLRYVVPTEASNLWFDNMVIPKTVKNQNSAYAF
INFMLKPENALQNAEYVGYSTPNLPAKELLPEETKEDKAFYPDVETMKHLEVYEKFDHKWTGKYSDLFLQ
FKMYRK
encoded by the CDS
ATGAAAAAAATCTATTCATTTTTAGCAGGAATTGCAGCGATTATCCTTGTCTTGTGGGGAATTGCGACTC
ATTTAGATAGTAAAATCAATAGTCGAGATAGTCAAAAATTGGTTATCTATAACTGGGGAGACTATATCGA
TCCTGAACTCTTGACTCAGTTTACAGAAGAAACAGGAATTCAAGTTCAGTACGAGACTTTTGACTCCAAC
GAAGCCATGTACACTAAGATAAAGCAGGGTGGAACGACCTACGATATTGCCATTCCAAGTGAATACATGA
TTAACAAGATGAAGGACGAAGACCTCTTGGTTCCGCTTGATTATTCAAAAATTGAAGGAATCGAAAATAT
CGGACCAGAGTTTCTCAACCAGTCCTTTGACCCAGGTAATAAATTCTCCATCCCTTACTTCTGGGGAACC
TTAGGAATTGTCTACAACGAAACCATGGTAGATGAAGCGCCTGAGCATTGGGATGACCTTTGGAAGCCGG
AGTATAAGAATTCTATCATGCTCTTTGATGGGGCGCGTGAGGTGCTGGGACTAGGACTCAATTCCCTCGG
CTACAGCCTCAACTCCAAGGATCTGCAGCAGTTGGAAGAGACAGTGGATAAGCTCTACAAACTGACTCCA
AATATCAAGGCTATCGTTGCGGACGAGATGAAGGGCTATATGATTCAGAATAATGTTGCAATCGGCGTGA
CCTTCTCTGGTGAAGCCAGCCAAATGTTAGAAAAAAATGAAAATCTACGTTATGTGGTACCGACAGAGGC
CAGCAATCTTTGGTTTGACAATATGGTCATTCCCAAAACAGTTAAAAACCAAAACTCAGCCTATGCCTTT
ATCAACTTTATGTTGAAACCTGAAAATGCTCTCCAAAATGCGGAGTATGTCGGCTATTCAACACCAAACC
TACCAGCGAAGGAATTGCTCCCAGAGGAAACAAAGGAAGATAAGGCCTTCTATCCCGATGTTGAAACCAT
GAAACACCTAGAAGTTTATGAGAAATTTGACCATAAATGGACAGGGAAATATAGCGACCTCTTCCTACAG
TTTAAAATGTATCGGAAGTAG

SP_{—}0082

The hypotetical protein SP_{—}0082 (Table XIVa) has been described as Cell wall surface anchor family protein with a Molecular Weight 90924 Da and protein length 857 AA. The UniProtKB/TrEMBL entry name is Q97T70_STRPN and primary accession number Q97T70. It has been described in Tettelin H., et al. RL Science 293:498-506(2001). Two fragments thereof SP4 (Table XIVb) and SP7 (Table XIVc) where used in the immunization experiments (infra).

TABLE XIVa

MKFNPNQRYTRWSIRRLSVGVASVVVASGFFVLVGQPSSVRADGLNPTPGQVLPEETSGT
KEGDLSEKPGDTVLTQAKPEGVTGNTNSLPTPTERTEVSEETSPSSLDTLFEKDEEAQKN
PELTDVLKETVDTADVDGTQASPAETTPEQVKGGVKENTKDSIDVPAAYLEKAEGKGPFT
AGVNQVIPYELFAGDGMLTRLLLKASDNAPWSDNGTAKNPALPPLEGLTKGKYFYEVDLN
GNTVGKQGQALIDQLRANGTQTYKATVKVYGNKDGKADLTNLVATKNVDININGLVAKET
VQKAVADNVKDSIDVPAAYLEKAKGEGPFTAGVNHVIPYELFAGDGMLTRLLLKASDKAP
WSDNGDAKNPALSPLGENVKTKGQYFYQVALDGNVAGKEKQALIDQFRANGTQTYSATVN
VYGNKDGKPDLDNIVATKKVTININGLISKETVQKAVADNVKDSIDVPAAYLEKAKGEGP
FTAGVNHVIPYELFAGDGMLTRLLLKASDKAPWSDNGDAKNPALSPLGENVKTKGQYFYQ
LALDGNVAGKEKQALIDQFRANGTQTYSATVNVYGNKDGKPDLDNIVATKKVTININGLI
SKETVQKAVADNVKDSIDVPAAYLEKAKGEGPFTAGVNHVIPYELFAGDGMLTRLLLKAS
DKAPWSDNGDAKNPALSPLGENVKTKGQYFYQVALDGNVAGKEKQALIDQFRANGTQTYS
ATVNVYGNKDGKPDLDNIVATKKVTIKINVKETSDTANGSLSPSNSGSGVTPMNHNHATG
TTDSMPADTMTSSTNTMAGENMAASANKMSDTMMSEDKAMLPNTGETQTSMASIGFLGLA
LAGLLGGLGLKNKKEEN
encoded by the CDS
ATGAAATTCAATCCAAATCAAAGATATACTCGTTGGTCTATTCGCCGTCTCAGTGTCGGT
GTTGCCTCAGTTGTTGTGGCTAGTGGCTTCTTTGTCCTAGTTGGTCAGCCAAGTTCTGTA
CGTGCCGATGGGCTCAATCCAACCCCAGGTCAAGTCTTACCTGAAGAGACATCGGGAACG
AAAGAGGGTGACTTATCAGAAAAACCAGGAGACACCGTTCTCACTCAAGCGAAACCTGAG
GGCGTTACTGGAAATACGAATTCACTTCCGACACCTACAGAAAGAACTGAAGTGAGCGAG
GAAACAAGCCCTTCTAGTCTGGATACACTTTTTGAAAAAGATGAAGAAGCTCAAAAAAAT
CCAGAGCTAACAGATGTCTTAAAAGAAACTGTAGATACAGCTGATGTGGATGGGACACAA
GCAAGTCCAGCAGAAACTACTCCTGAACAAGTAAAAGGTGGAGTGAAAGAAAATACAAAA
GACAGCATCGATGTTCCTGCTGCTTATCTTGAAAAAGCTGAAGGGAAAGGTCCTTTCACT
GCCGGTGTAAACCAAGTAATTCCTTATGAACTATTCGCTGGTGATGGTATGTTAACTCGT
CTATTACTAAAAGCTTCGGATAATGCTCCTTGGTCTGACAATGGTACTGCTAAAAATCCT
GCTTTACCTCCTCTTGAAGGATTAACAAAAGGGAAATACTTCTATGAAGTAGACTTAAAT
GGCAATACTGTTGGTAAACAAGGTCAAGCTTTAATTGATCAACTTCGCGCTAATGGTACT
CAAACTTATAAAGCTACTGTTAAAGTTTACGGAAATAAAGACGGTAAAGCTGACTTGACT
AATCTAGTTGCTACTAAAAATGTAGACATCAACATCAATGGATTAGTTGCTAAAGAAACA
GTTCAAAAAGCCGTTGCAGACAACGTTAAAGACAGTATCGATGTTCCAGCAGCCTACCTA
GAAAAAGCCAAGGGTGAAGGTCCATTCACAGCAGGTGTCAACCATGTGATTCCATACGAA
CTCTTCGCAGGTGATGGCATGTTGACTCGTCTCTTGCTCAAGGCATCTGACAAGGCACCA
TGGTCAGATAACGGCGACGCTAAAAACCCAGCCCTATCTCCACTAGGCGAAAACGTGAAG
ACCAAAGGTCAATACTTCTATCAAGTAGCCTTGGACGGAAATGTAGCTGGCAAAGAAAAA
CAAGCGCTCATTGACCAGTTCCGAGCAAATGGTACTCAAACTTACAGCGCTACAGTCAAT
GTCTATGGTAACAAAGACGGTAAACCAGACTTGGACAACATCGTAGCAACTAAAAAAGTC
ACTATTAACATAAACGGTTTAATTTCTAAAGAAACAGTTCAAAAAGCCGTTGCAGACAAC
GTTAAAGACAGTATCGATGTTCCAGCAGCCTACCTAGAAAAAGCCAAGGGTGAAGGTCCA
TTCACAGCAGGTGTCAACCATGTGATTCCATACGAACTCTTCGCAGGTGATGGTATGTTG
ACTCGTCTCTTGCTCAAGGCATCTGACAAGGCACCATGGTCAGATAACGGTGACGCTAAA
AACCCAGCCCTATCTCCACTAGGTGAAAACGTGAAGACCAAAGGTCAATACTTCTATCAA
TTAGCCTTGGACGGAAATGTAGCTGGCAAAGAAAAACAAGCGCTCATTGACCAGTTCCGA
GCAAACGGTACTCAAACTTACAGCGCTACAGTCAATGTCTATGGTAACAAAGACGGTAAA
CCAGACTTGGACAACATCGTAGCAACTAAAAAAGTCACTATTAACATAAACGGTTTAATT
TCTAAAGAAACAGTTCAAAAAGCCGTTGCAGACAACGTTAAGGACAGTATCGATGTTCCA
GCAGCCTACCTAGAAAAGGCCAAGGGTGAAGGTCCATTCACAGCAGGTGTCAACCATGTG
ATTCCATACGAACTCTTCGCAGGTGATGGCATGTTGACTCGTCTCTTGCTCAAGGCATCT
GACAAGGCACCATGGTCAGATAACGGCGACGCTAAAAACCCAGCTCTATCTCCACTAGGT
GAAAACGTGAAGACCAAAGGTCAATACTTCTATCAAGTAGCCTTGGACGGAAATGTAGCT
GGCAAAGAAAAACAAGCGCTCATTGACCAGTTCCGAGCAAACGGTACTCAAACTTACAGC
GCTACAGTCAATGTCTATGGTAACAAAGACGGTAAACCAGACTTGGACAACATCGTAGCA
ACTAAAAAAGTCACTATTAAGATAAATGTTAAAGAAACATCAGACACAGCAAATGGTTCA
TTATCACCTTCTAACTCTGGTTCTGGCGTGACTCCGATGAATCACAATCATGCTACAGGT
ACTACAGATAGCATGCCTGCTGACACCATGACAAGTTCTACCAACACGATGGCAGGTGAA
AACATGGCTGCTTCTGCTAACAAGATGTCTGATACGATGATGTCAGAGGATAAAGCTATG
CTACCAAATACTGGTGAGACTCAAACATCAATGGCAAGTATTGGTTTCCTTGGGCTTGCG
CTTGCAGGTTTACTCGGTGGTCTAGGTTTGAAAAACAAAAAAGAAGAAAACTAA

TABLE XIVb

DVDGTQASPAETTPEQVKGGVKENTKDSIDVPAAYLEKAEGKGPFTAGVNQVIPYELFAGDGMLTRLLLKASDNAPW
SDNGTAKNPALPPLEGLTKGKYFYEVDLNGNTVGKQGQALIDQLRANGTQTYKATVKVYGNKDGKADLTNLVATKNV
DININGLVAKETVQKAVADNVKDSIDVPAAYLEKAKGEGPFTAGVN
CDS
GATGTGGATGGGACACAAGCAAGTCCAGCAGAAACTACTCCTGAACAAGTAAAAGGTGGAGTGAAAGAAAATACAAA
AGACAGCATCGATGTTCCTGCTGCTTATCTTGAAAAAGCTGAAGGGAAAGGTCCTTTCACTGCCGGTGTAAACCAAG
TAATTCCTTATGAACTATTCGCTGGTGATGGTATGTTAACTCGTCTATTACTAAAAGCTTCGGATAATGCTCCTTGG
TCTGACAATGGTACTGCTAAAAATCCTGCTTTACCTCCTCTTGAAGGATTAACAAAAGGGAAATACTTCTATGAAGT
AGACTTAAATGGCAATACTGTTGGTAAACAAGGTCAAGCTTTAATTGATCAACTTCGCGCTAATGGTACTCAAACTT
ATAAAGCTACTGTTAAAGTTTACGGAAATAAAGACGGTAAAGCTGACTTGACTAATCTAGTTGCTACTAAAAATGTA
GACATCAACATCAATGGATTAGTTGCTAAAGAAACAGTTCAAAAAGCCGTTGCAGACAACGTTAAAGACAGTATCGA
TGTTCCAGCAGCCTACCTAGAAAAAGCCAAGGGTGAAGGTCCATTCACAGCAGGTGTCAAC

TABLE XIVc

EKQALIDQFRANGTQTYSATVNVYGNKDGKPDLDNIVATKKVTININGLISKETVQKAVADNVKDSIDVPAAYLEKA
KGEGPFTAGVNHVIPYELFAGDGMLTRLLLKASDKAPWSDNGDAKNPALSPLGENVKTKGQYFYQVALDGNVAGKEK
QALIDQFRANGTQTYSATVNVYGNKDGKPDLDNIVATKKVTIKINVKETSDTANGSLSPSNSGSGVTPMNHNHATGT
TDSMPADTMTSSTNTMAGENMAASANKMSDTMMSEDKAMLPNTGETQT
CDS
AAGAAAAACAAGCGCTCATTGACCAGTTCCGAGCAAACGGTACTCAAACTTACAGCGCTACAGTCAATGTCTATGGT
AACAAAGACGGTAAACCAGACTTGGACAACATCGTAGCAACTAAAAAAGTCACTATTAACATAAACGGTTTAATTTC
TAAAGAAACAGTTCAAAAAGCCGTTGCAGACAACGTTAAGGACAGTATCGATGTTCCAGCAGCCTACCTAGAAAAGG
CCAAGGGTGAAGGTCCATTCACAGCAGGTGTCAACCATGTGATTCCATACGAACTCTTCGCAGGTGATGGCATGTTG
ACTCGTCTCTTGCTCAAGGCATCTGACAAGGCACCATGGTCAGATAACGGCGACGCTAAAAACCCAGCTCTATCTCC
ACTAGGTGAAAACGTGAAGACCAAAGGTCAATACTTCTATCAAGTAGCCTTGGACGGAAATGTAGCTGGCAAAGAAA
AACAAGCGCTCATTGACCAGTTCCGAGCAAACGGTACTCAAACTTACAGCGCTACAGTCAATGTCTATGGTAACAAA
GACGGTAAACCAGACTTGGACAACATCGTAGCAACTAAAAAAGTCACTATTAAGATAAATGTTAAAGAAACATCAGA
CACAGCAAATGGTTCATTATCACCTTCTAACTCTGGTTCTGGCGTGACTCCGATGAATCACAATCATGCTACAGGTA
CTACAGATAGCATGCCTGCTGACACCATGACAAGTTCTACCAACACGATGGCAGGTGAAAACATGGCTGCTTCTGCT
AACAAGATGTCTGATACGATGATGTCAGAGGATAAAGCTATGCTACCAAATACTGGTGAGACTCAAACA

SP_{—}0965

The hypotetical protein SP_{—}0965 (Table XV) has been described as Putative endo-beta-N-acetylglucosaminidase with a Molecular Weight 76469 Da and protein length 658 AA. The UniProtKB/TrEMBL entry name is LYTB_STRPN and primary accession number P59205. It has been described in Tettelin H., et al. RL Science 293:498-506(2001). The recombinant protein used in the immunization experiments lacked the underlined fragment.

TABLE XV

MKKVRFIFLALLFFLASPEGAMASDGTWQGKQYLKEDGSQAANEWVFDTHYQSWFYIKADANYAENEWLKQGDDYFY
LKSGGYMAKSEWVEDKGAFYYLDQDGKMKRNAWVGTSYVGATGAKVIEDWVYDSQYDAWFYIKADGQHAEKEWLQIK
GKDYYFKSGGYLLTSQWINQAYVNASGAKVQQGWLFDKQYQSWFYIKENGNYADKEWIFENGHYYYLKSGGYMAANE
WIWDKESWFYLKFDGKMAEKEWVYDSHSQAWYYFKSGGYMTANEWIWDKESWFYLKSDGKIAEKEWVYDSHSQAWYY
FKSGGYMTANEWIWDKESWFYLKSDGKIAEKEWVYDSHSQAWYYFKSGGYMAKNETVDGYQLGSDGKWLGGKTTNEN
AAYYQVVPVTANVYDSDGEKLSYISQGSVVWLDKDRKSDDKRLAITISGLSGYMKTEDLQALDASKDFIPYYESDGH
RFYHYVAQNASIPVASHLSDMEVGKKYYSADGLHFDGFKLENPFLFKDLTEATNYSAEELDKVFSLLNINNSLLENK
GATFKEAEEHYHINALYLLAHSALESNWGRSKIAKDKNNFFGITAYDTTPYLSAKTFDDVDKGILGATKWIKENYID
RGRTFLGNKASGMNVEYASDPYWGEKIASVMMKINEKLGGKD
CDS
ATGAAGAAAGTAAGATTTATTTTTTTAGCTCTGCTATTTTTCTTAGCTAGTCCAGAGGGTGCAATGGCTA
GTGATGGTACTTGGCAAGGAAAACAGTATCTGAAAGAAGATGGCAGTCAAGCAGCAAATGAGTGGGTTTT
TGATACTCATTATCAATCTTGGTTCTATATAAAAGCAGATGCTAACTATGCTGAAAATGAATGGCTAAAG
CAAGGTGACGACTATTTTTACCTCAAATCTGGTGGCTATATGGCCAAATCAGAATGGGTAGAAGACAAGG
GAGCCTTTTATTATCTTGACCAAGATGGAAAGATGAAAAGAAATGCTTGGGTAGGAACTTCCTATGTTGG
TGCAACAGGTGCCAAAGTAATAGAAGACTGGGTCTATGATTCTCAATACGATGCTTGGTTTTATATCAAA
GCAGATGGACAGCACGCAGAGAAAGAATGGCTCCAAATTAAAGGGAAGGACTATTATTTCAAATCCGGTG
GTTATCTACTGACAAGTCAGTGGATTAATCAAGCTTATGTGAATGCTAGTGGTGCCAAAGTACAGCAAGG
TTGGCTTTTTGACAAACAATACCAATCTTGGTTTTACATCAAAGAAAATGGAAACTATGCTGATAAAGAA
TGGATTTTCGAGAATGGTCACTATTATTATCTAAAATCCGGTGGCTACATGGCAGCCAATGAATGGATTT
GGGATAAGGAATCTTGGTTTTATCTCAAATTTGATGGGAAAATGGCTGAAAAAGAATGGGTCTACGATTC
TCATAGTCAAGCTTGGTACTACTTCAAATCCGGTGGTTACATGACAGCCAATGAATGGATTTGGGATAAG
GAATCTTGGTTTTATCTCAAATCTGATGGGAAAATAGCTGAAAAAGAATGGGTCTACGATTCTCATAGTC
AAGCTTGGTACTACTTCAAATCCGGTGGTTACATGACAGCCAATGAATGGATTTGGGATAAGGAATCTTG
GTTTTACCTCAAATCTGATGGGAAAATAGCTGAAAAAGAATGGGTCTACGATTCTCATAGTCAAGCTTGG
TACTACTTCAAATCTGGTGGCTACATGGCGAAAAATGAGACAGTAGATGGTTATCAGCTTGGAAGCGATG
GTAAATGGCTTGGAGGAAAAACTACAAATGAAAATGCTGCTTACTATCAAGTAGTGCCTGTTACAGCCAA
TGTTTATGATTCAGATGGTGAAAAGCTTTCCTATATATCGCAAGGTAGTGTCGTATGGCTAGATAAGGAT
AGAAAAAGTGATGACAAGCGCTTGGCTATTACTATTTCTGGTTTGTCAGGCTATATGAAAACAGAAGATT
TACAAGCGCTAGATGCTAGTAAGGACTTTATCCCTTATTATGAGAGTGATGGCCACCGTTTTTATCACTA
TGTGGCTCAGAATGCTAGTATCCCAGTAGCTTCTCATCTTTCTGATATGGAAGTAGGCAAGAAATATTAT
TCGGCAGATGGCCTGCATTTTGATGGTTTTAAGCTTGAGAATCCCTTCCTTTTCAAAGATTTAACAGAGG
CTACAAACTACAGTGCTGAAGAATTGGATAAGGTATTTAGTTTGCTAAACATTAACAATAGCCTTTTGGA
GAACAAGGGCGCTACTTTTAAGGAAGCCGAAGAACATTACCATATCAATGCTCTTTATCTCCTTGCCCAT
AGTGCCCTAGAAAGTAACTGGGGAAGAAGTAAAATTGCCAAAGATAAGAATAATTTCTTTGGCATTACAG
CCTATGATACGACCCCTTACCTTTCTGCTAAGACATTTGATGATGTGGATAAGGGAATTTTAGGTGCAAC
CAAGTGGATTAAGGAAAATTATATCGATAGGGGAAGAACTTTCCTTGGAAACAAGGCTTCTGGTATGAAT
GTGGAATATGCTTCAGACCCTTATTGGGGCGAAAAAATTGCTAGTGTGATGATGAAAATCAATGAGAAGC
TAGGTGGCAAAGATTAG

To further validate the immunogenic potential of the identified S. pneumoniae proteins, we checked serum from patients with invasive pneumococcal disease for the presence of antibodies specific for one or more of the S. pneumoniae antigens of the present invention.

### Example 6

### Production of Recombinant Pneumococcal Proteins

6.1 Isolation of Genomic S. Pneumoniae DNA

200 μL S. pneumoniae serotype 4 (a kind gift of Prof. J. Verhaegen, nr 080260) was grown overnight at 37° C. (225 rpm) in 5 mL Todd Hewitt medium. The next day genomic DNA was isolated with Wizard Genomic DNA purification kit (Promega) as described by the supplier.

6.2 Gateway Cloning (Invitrogen)

PCR

SP_{—}1683, SP_{—}0562, SP_{—}0965 and SP_{—}1386 fragments were generated by PCR. In a total reaction volume of 25μl, 22 μL AccuPrime™ Pfx SuperMix (Invitrogen), 1 μL corresponding forwards primer (10 mM, table 1), 1 μL corresponding reverse primer (10 mM, table 1) and 1 μL genomic DNA (10-200 ng) were combined. PCR reactions were run on a 9700 thermocycler (Applied Biosystems) using the following conditions: 95 C for 5 min, then 35 cycles of 95° C. for 15 s, 55° C. for 30 s, and 68° C. for 3.30 min, followed by 72° C. for 5 min and then held at 4° C. As control five μL of the resulting PCR products were run on a 1% agarose gel. The obtained PCR products were purified by a GenElut™ PCR Clean-Up Kit (Sigma) as described by the supplier and checked on a 1% agarose gel.

TABLE 1

Corresponding forwards and reverse primers for SP_1683, SP_0562,
SP_0965 and SP_1386 fragments.

| | | |
|---|---|---|
| Name | Forwards | Reverse |

| | | |
|---|---|---|
| SP_1683 | GGGG-ACA-AGT-TTG-TAC-AAA-AAA-GCA- | GGGG-AC-CAC-TTT-GTA-CAA-GAA-AGC-TGG- |
| | GGC-TTC-AGTAAAGATGCTGCCAAATCAG | GTT-CTATTGTTTCATAGCTTTTTTGATTG |
| SP_0562 | GGGG-ACA-AGT-TTG-TAC-AAA-AAA-GCA- | GGGG-AC-CAC-TTT-GTA-CAA-GAA-AGC-TGG- |
| | GGC-TTC- | GTT-TTATTCTAATCCACGAAAATAGTCC |
| | ACAGAGTTTGAAAGTATGATTTTCAAG | |
| SP_0965 | GGGG-ACA-AGT-TTG-TAC-AAA-AAA-GCA- | GGGG-AC-CAC-TTT-GTA-CAA-GAA-AGC-TGG- |
| | GGC-TTC- | GTT-CTAATCTTTGCCACCTAGCTTCTCATTG |
| | AAAAATGAGACAGTAGATGGTTATCAG | |
| SP_1386 | GGGG-ACA-AGT-TTG-TAC-AAA-AAA-GCA- | GGGG-AC-CAC-TTT-GTA-CAA-GAA-AGC-TGG- |
| | GGC-TTC-TTAGATAGTAAAATCAATAG | GTT-CTACTTCCGATACATTTTAAACTGTAG |

‘BP’-Reaction

The PCR-products were inserted in a pDONR221 vector as described by the supplier. Subsequently a transformation of the BP reaction (see below) was performed in DH5α TM cells. The DH5α cells were plated at kanamycin selective LB-agar and overnight incubated at 37° C. The next day, one colony was inoculated in 5 mL DYT media containing 5 μL kanamycin and hold overnight at 37° C. (three times, 225 rpm). The plasmids were isolated with the GenElute™ Plasmid MiniPrep Kit (Sigma) as described by the supplier. To check the insert a PCR was performed. In a total reaction volume of 25 μl, 22 μL AccuPrime™ Pfx SuperMix (Invitrogen), 1 μL M13 forwards primer (10 mM), 1 μL M13 reverse primer (10 mM) and 1 μL plasmid DNA were combined. PCR reactions were run on a 9700 thermocycler (Applied Biosystems) using the following conditions: 95 C for 5 min, then 35 cycles of 95° C. for 15 s, 55° C. for 30 s, and 68° C. for 3.30 min, followed by 72° C. for 5 min and then held at 4° C. As control five μL of the resulting PCR products were run on a 1% agarose gel.

‘LR’-Reaction

The insert of the pDONR221 vector was exchanged to a destination vector (pDEST™ 17, Invitrogen) as described by the supplier. Subsequently a transformation of the BP reaction (see below) was performed in DH5α TM cells. The DH5α cells were plated at ampicillin selective LB-agar and overnight incubated at 37° C. The next day, one colony (three times) was inoculated in 5 mL DYT media containing 5 μL ampicillin and hold overnight at 37° C. (225 rpm). The plasmids were isolated with the GenElute™ Plasmid MiniPrep Kit (Sigma) as described by the supplier. To check the insert a PCR was performed. In a total reaction volume of 25 μl, 22 μL AccuPrime™ Pfx SuperMix (Invitrogen), 1 μL T7 forwards primer (10 mM), 1 μL T7 reverse primer (10 mM) and 1 μL plasmid DNA were combined. PCR reactions were run on a 9700 thermocycler (Applied Biosystems) using the following conditions: 95 C for 5 min, then 35 cycles of 95° C. for 15 s, 55° C. for 30 s, and 68° C. for 3.30 min, followed by 72° C. for 5 min and then held at 4° C. As control five μL of the resulting PCR products were run on a 1% agarose gel.

Transformation to Competent E. coli

200 μL E. coli (KRK, BL21 Al or DH5α cells, Invitrogen), carefully thaw out on ice, were combined with 10 μL plasmide DNA and incubated on ice for 5 min After a heat-shock at 42° C. for 20 s, the cells were incubate on ice for 2 min. 100 μL DYT medium was add, cells were hold at 37° C. for one hour and then plated at selective LB-agar and overnight incubated at 37° C.

Induction of Recombinant Protein Production

20 μL Krk or BL21 E. coli glycerol stock were inoculated in 20 mL DYT medium (ampicillin, overnight, 37° C.). The next day, 20 mL culture was transferred to 800 mL medium (ampicillin) and hold at 37° C. until the optical density (OD₆₀₀) reach 0.6-0.8. The protein production was induced on there the conditions as described in table 2. At the end of the induction PMSF (200 mM) and Benzamidin (65 mM) were added to reduce protein degradation. The cultures were centrifugated (10000 rpm, Sorvall RC 6C Plus, DuPont) at 4° C. for 10 min. The cell pellet was collected and hold at −80° C.

TABLE 2
Induction conditions:

| | MW | | | | |
|---|---|---|---|---|---|
| Name | (KDa) | E. coli | Induction | location | purification |
| SP_1683 | 46 | Krk cells | IPTG (1 mM), 6 h, 37° C. | cytoplasm | HIS-tag, NI-NTA agarose |
| SP4* | 40 | Krk cells | IPTG (1 mM), 6 h, 37° C. | cytoplasm | GST-tag, Aëkta |
| SP17* | 46 | Krk cells | IPTG (1 mM), 6 h, 37° C. | cytoplasm | GST-tag, Åëkta |
| SP_0562 | 28 | BL21 cells | 0.2% arabinose, 4 h, 28° C. | Inclusion | HIS-tag, NI-NTA agarose |
| | | | | bodies | |
| SP_0965 | 33 | BL21 cells** | 0.2% arabinose, 6 h, 23° C. | Inclusion | HIS-tag, NI-NTA agarose |
| | | | | bodies | |
| SP_1386 | 38 | BL21 cells | 0.2% arabinose, 4 h, 28° C. | Inclusion | HIS-tag, NI-NTA agarose |
| | | | | bodies | |

*Clones were obtained from Prof. Franco Felicl, Kenton Srl, Pomezia, Rome, Italy.
**The LB-agar plates and the 20 mL DYT start culture for this protein contain 0.1% glucose to reduce the basal metabolism.

NI-NTA Agarose/Aekta Purification and Dialysis.

The cell pellet which contains the recombinant protein in the cytoplasm, was resuspended in 20 mL lysisbuffer (50 mM NaH₂PO₄, 0.3 M NaCl, 40 mM Imidazol, 0.13 mM PMSF, 0.83 mM Benzamidin, protease inhibitor tablet (Roche)) and 1 mL 20% Triton (Sigma), sonicated (9×10 s at 12 kHz, with 5 s off) and centrifugated for 30 min at 12000 rpm (Sorvall RC 6C Plus, DuPont) at 4° C. The cell pellet which contains the recombinant protein in inclusion bodies, was resuspended in 20 mL inclusion body sonication buffer (25 mM HEPES, pH 7.7, 100 mm KCl, 12.5 mM MgCl₂, 20% glycerol, 0.1 (v/v) Nonidet P-40, 1 mM DTT, protease inhibitor tablet (Roche), 5 mg lysozym) and incubated on ice for 30 min and 60 min at −80° C. The suspension was sonicated (8×15 s at 12 kHz, with 15 s off), and centrifugated for 10 min at 10000 rpm (Sorvall RC 6C Plus, DuPont) at 4° C. The pellet was washed with 5 mL RIPA buffer (4° C., 0.1% SDS, 1% Triton, 1% sodium deoxycholate in TBS [25 mM Tris-HCl pH 7.5, 150 mM NaCl]) and centrifugated again. The obtained pellet was resuspended in 3 mL Buffer A (100 mM NaH₂PO₄, 10 mM Tris-HCl, 8 M urea, pH 8).

Recombinant proteins were isolated and purified with NI-NTA agarose (Invitrogen) as described . by the supplier. GST-fusion proteins were purified via Aëkta Fast protein Liquid chromatography. 1D SDS electrophoresis (GH Healthcare) was performed to control the elution fractions and the fractions containing the recombinant protein were dialysis to PBS (3×1 h, 1 L, 4° C.) in Slide-A-Lyzer Dialysis Cassettes (10-25 KDa MWCO, Thermo scientific).

### Example 7

### Immunoblotting with Convalescent Patient Serum

500 ng recombinant protein (rSP_{—}1683, rSP_{—}0562, recombinant fragments SP4 and SP17, rSP_{—}0965, rSP_{—}1386 and rPspA) obtained as provided in Example 6 was loaded on a 12.5% SDS polyacrylamide gel for separation according to the molecular weight. After separation the western blotting as described above (Example 4) was performed although the membranes were now overnight incubated (diluted in trissaline buffer, 1/500) with convalescent serum (day 14—day 90 after infection, n=8) obtained from patients with invasive pneumococcal disease or with serum obtained from healthy donors (n=3).

Results

The presents of human antibodies against the recombinant pneumococcal proteins in convalescent human serum was tested with immunoblotting technology. The serum was obtained from patients survived a invasive pneumococcal infection (between 7 days and 90 days after infection). As positive controls serum obtained from healthy donors (n=3) was used. The healthy controls produced antibodies against the 7 tested recombinant proteins and the total S. pneumoniae extract. Also the convalescent human sera contained antibodies against the 7 tested recombinant proteins and the total S. pneumoniae extract. Although one convalescent human serum did not contain antibodies against rSP_{—}1683. The data are presented in table 3. These results confirm the immunogenicity of the tested pneumococcal proteins.

TABLE 3
Human antibody responses against recombinant SP_1683, SP_0562, SP4, SP17, SP_0965,
SP_1386, S. pneumoniae serotype 3 extract (HK3 extract) and PspA.

| | Name | | | | | | | |
|---|---|---|---|---|---|---|---|---|
| | rSP_1683 | rSP_0562 | SP4 | SP17 | rSP_0965 | rSP_1386 | HK3 extract | rPspA |
| Patients (n = 8) | 7 | 8 | 8 | 8 | 8 | 8 | 8 | 8 |
| Control (n = 3) | 3 | 3 | 3 | 3 | 3 | 3 | 3 | 3 |

The humanized SCID/SCID model described in this application can also be used to evaluate the protective capacity of the antibodies, the advantage being that no healthy volunteers are needed for initial vaccination studies.

We identified several cytoplasmic pneumococcal proteins as antigens after immunization with intact heat inactivated S. pneumoniae. This might be a bias resulting from the release of cytoplasmic components during the procedure of heat killing. On the other hand, Pancholi et al. described that several housekeeping enzymes can be found on the surface of pathogens. In many pathogens, certain housekeeping enzymes play a role in enhancing virulence. To perform such a function, enzymes must be located on the surface of the pathogens. Although they do not have the typical signal sequence or membrane anchoring mechanisms, they do get secreted and are displayed on the surface, probably by re-association. Once on the surface, these enzymes interact with host components, such as fibronectin and plasminogen, or interact directly with the host cells to trigger signal transduction. In this way, housekeeping enzymes may enable pathogens to colonize, persist and invade the host tissue. Therefore, certain housekeeping enzymes may act as putative virulence factors and are potential targets for the development of new strategies to control infection by using agents that can block their secretion and/or re-association (Pancholi V., G. S. Chhatwal. 2003. Int J Med Microbiol. 293: 391-401). Additionally, S. pneumoniae contains a self-destructive enzyme, autolysin. The action of autolysin may lead to cell lysis (Jedrzejas M. J. 2001. Microbiol. Mol. Biol. Rev. 65(2): 187-207). Therefore, it is not unusual that individuals face cytoplasmatic pneumococcal components during the course of pneumococcal colonization and/or invasion.

One should also consider that overnight breeding of the bacteria at 37° C. can cause increased expression of HSPs. But, pneumococci also experience heat shock in the host during penetration from the nasopharyngeal niche (30 to 34° C.) into the bloodstream (37° C.), and this change can trigger a rapid and transient increase in the levels of HSPs (Kwon H. Y., et al. 2003. Infect. Immun. 71(7): 3757-3765). Based on their cytoplasmatic location and absence of protection capacity in mice, it is doubtful that DnaK and probably also GroEL will be excellent pneumococcal vaccine candidates. On the other hand, our finding that CpP is immunogenic supports the recent increased interest towards this pneumococcal protein (Kwon H.-Y., A. D. et al. 2004. Infect. Immun. 72(10): 5646-5653, Cao J., et al. 2007. Vaccine 25(27): 4996-5005).

Using a humanized SCID/SCID mice model and a proteomics approach we described a whole array of pneumococcal proteins that are immunogenic for the human immune system after immunization with intact heat-inactivated S. pneumoniae.

These antigens can be used in a protein vaccin to prevent pneumoccal infections.

Preparation of Vaccinating Agents: Vaccinating agents of the present invention can be synthesized chemically (see, e.g., Beachey et al., Nature 292: 457-459, 1981), or generated recombinantly.

The vaccinating agent can be constructed so as to contain a selective portion that can be lost or cleaved in vivo without affecting the efficacy of the vaccine. This may be accomplished by, for example, including an inconsequential non-immunogenic polypeptide at the end, or, including an immunogenic polypeptide that does not adversely impact the efficiency of the vaccine (e.g., a reiterated immunogenic polypeptide may be included at the end of the vaccine). Furthermore; protective antigens from unrelated pathogens can also be combined into a single polypeptide, which may circumvent the need for carriers. Vaccines against some pathogens might include T and B cell epitopes originally derived from different disease related S. pneumoniae proteins on the same hybrid construct. Additionally, multivalent hybrid proteins may be sufficient conjugates in carbohydrate vaccines.

For protein expression, the multivalent genes are ligated into any suitable replicating vector (e.g. plasmid) which is used to transform an appropriate prokaryote host strain. Prokaryotes include gram negative or gram positive organisms, for example E. coli or bacilli. Suitable prokaryotic hosts cells for transformation include, for example, E. coli, Bacillus subtilis, Salmonella typhimurium, and various other species within the genera Pseudomonas, Streptomyces, and Staphylococcus.

Expression vectors transfected into prokaryotic host cells generally comprise one or more phenotypic selectable markers such as, for example, a gene encoding proteins that confer antibiotic resistance or that supplies an auxotrophic requirement, and an origin of replication recognized by the host to ensure amplification within the host. Other useful expression vectors for prokaryotic host cells include a selectable marker of bacterial origin derived from commercially available plasmids. This selectable marker can comprise genetic elements of the cloning vector pBR322 (ATCC 37017). Briefly, pBR322 contains genes for ampicillin and tetracycline resistance and thus provides simple means for identifying transformed cells. The pBR322 “backbone” sections are combined with an appropriate promoter and a mammalian ETF structural gene sequence. Other commercially available vectors include, for example, pKK223-3 (Pharmacia Fine Chemicals, Uppsala, Sweden), pQE30 (His-tag expression vector), and pGEM1 (Promega Biotec, Madison, Wis., USA).

Common promoter sequences for use within prokaryotic expression vectors include β-lactamase (penicillinase), lactose promoter system (Chang et al., Nature 275: 615, 1978; and Goeddel et al., Nature 281: 544, 1979), tryptophan (trp) promoter system (Goeddel et al., Nucl. Acids Res. 8: 4057, 1980; and EPA 36,776) and tac promoter (Sambrook et al., Molecular Cloning: A Laboratory Manual, Cold Spring Harbor Laboratory, (1989)). A particularly useful prokaryotic host cell expression system employs a phage λ PL promoter and a c1857ts thermolabile repressor sequence. Plasmid vectors available from the American Type Culture Collection that incorporate derivatives of the λ PL promoter include plasmid pHUB2 (resident in E. coli strain JMB9 (ATCC 37092)) and pPLc28 (resident in E. coli RR1 (ATCC 53082)).

Transformation of the host strains of E. coli is accomplished by using standard methods (Dale et al., “Recombinant tetravalent group A streptococcal Streptococcus pneumoniae protein vaccine,” J. Immunol. 151: 2188-2194, 1993; Dale et al., “Recombinant, octavalent group A streptococcal Streptococcus pneumoniae protein vaccine,” Vaccine 14: 944-948, 1996).

The molecular size of the recombinant protein expressed by selected clones is determined by performing Western blots of extracts of E. coli (Dale et al., “Recombinant tetravalent group A streptococcal S. pneumoniae protein vaccine,” J. Immunol. 151: 2188-2194, 1993). The multivalent gene is sequenced by the dideoxy-nucleotide chain termination method to confirm that each gene fragment is an exact copy of the native selected sequence.

Also, correct recombinant protein production will be evaluated by Maldi-Tof-Tof analysis after purification of the recombinant protein.

### Example 8

### Immunisation of the Balb\c Mice and Humanised SCID/SCID Mice

Balb\c mice and humanised SCID/SCID mice (same day as transplant of human PBMC see example 3 above) were i.p. immunized with 25 μg dialyse recombinant pneumococcal protein (rSP_{—}1683,rSP_{—}0562, fragments SP4 and SP17, rSP_{—}0965, and rSP_{—}1386) in PBS containing 1 mg/mL Alum adjuvant (Sigma) on day 0 and were boosted with the same concentration on day 14. Blood was redrawn via retro-orbital punction in anesthetised (Isoflurane inhalation) mice on day 14 and day 28.

8.1. Challenge with S. pneumoniae Serotype 3

Immunized Balb\c and humanized SCID/SCID mice were challenged at day 28-32 i.p. with 10⁴ S. pneumoniae. The survival was monitored until day 7 after infection. Approval of the study was granted by the local ethics committee of the Catholic University Leuven.

Results Balb\c Mice

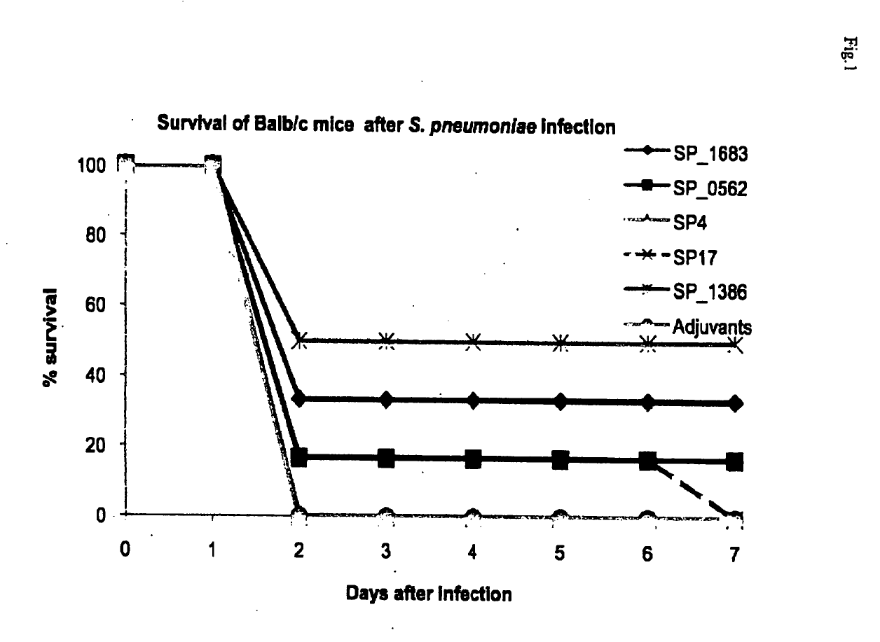

The control mice, which received only the adjuvant, and mice immunized with SP4 or SP17 died within the next two days after infection. One mouse immunized with SP17 died 6 days after infection. One, two and three out of the six mice immunized with SP_{—}0562, SP_{—}1683 and SP_{—}1386, respectively, were still alive seven days after infection. The data are presented in FIG. 1.

Results Humanized SCID/SCID

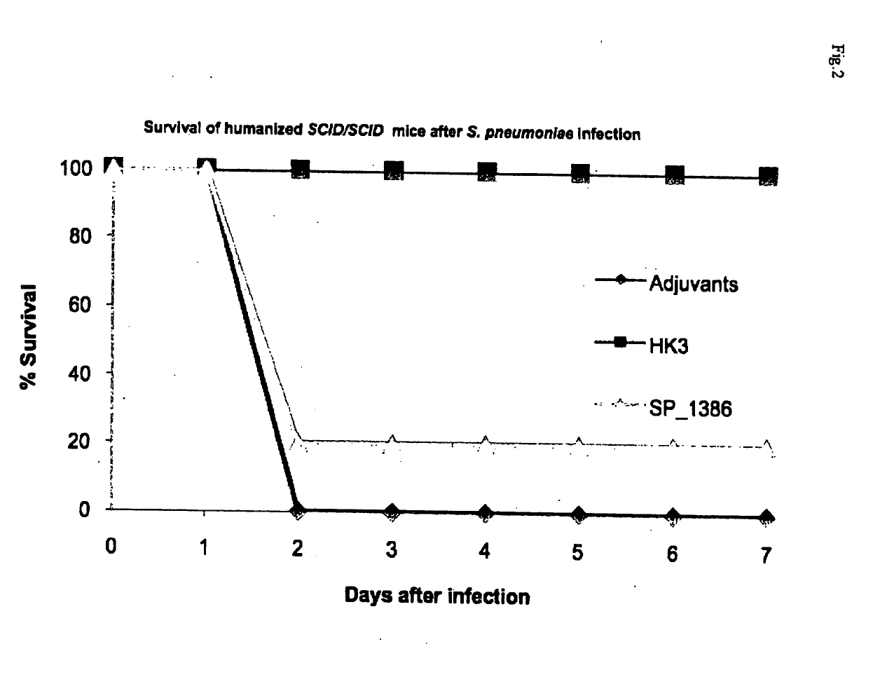

Humanized SCID/SCID mice were immunized i.p. with 25 μg rSP_{—}1386 in PBS containing 1 mg/mL Alum adjuvants (n=5), with the adjuvants (n=3), or with intact heat inactivated S. pneumoniae (HK3, serotype 3, n=3) on day 0 and boosted on day 14. On day 28 these mice were challenged with S. pneumoniae serotype 3 (10⁴ CFU). Two days after infection the control mice and four of the mice immunized with SP_{—}1386 were died. Mice immunized with heat inactivated S. pneumoniae and one mouse immunized with SP_{—}1386 survived the infection. The data are presented in FIG. 2.

Gene-delivery Vehicle-based Vaccines Injection of mammals with gene delivery vehicles (e.g., naked DNA) encoding antigens of various pathogens has been shown to result in protective immune responses (Ulmer et al., Science 259: 1745-9, 1993; Bourne et al., J. Infect. Dis. 173: 800-7, 1996; Hoffman et al., Vaccine 12: 1529-33, 1994). Since the original description of in vivo expression of foreign proteins from naked DNA injected into muscle tissue (Wolff et al., Science 247: 1465-8, 1990), there have been several advances in the design and delivery of DNA for purposes of vaccination.

The S. pneumoniae protein vaccines described above are ideally suited for delivery via naked DNA because protective immunity is ultimately determined by antibodies. For example, within one embodiment the multivalent genes are ligated into plasmids that are specifically engineered for mammalian cell expression (see, e.g., Hartikka et al., Hum Gene Ther 7: 1205-17, 1996, which contains the promoter/enhancer element from cytomegalovirus early gene, the signal peptide from human tissue plasminogen activator and a terminator element from the bovine growth hormone gene). The S. pneumoniae protein hybrid genes can be cloned into the plasmid which is used to transfect human cell lines to assure recombinant protein expression. The plasmid is propagated in E. coli and purified in quantities sufficient for immunization studies by cesium chloride gradient centrifugation. Mice are immunized with 50 ug of plasmid in 50 ul saline given intramuscularly into the rectus femoris. Booster injections of the same dose are given at three and six weeks after the initial injection.

A wide variety of other gene delivery vehicles can likewise be utilized within the context of the present invention, including for example, viruses, retrotransposons and cosmids. Representative examples include adenoviral vectors (e.g., WO 94/26914, WO 93/9191; Yei et al., Gene Therapy 1: 192-200, 1994; Kolls et al., PNAS 91(1): 215-219, 1994; Kass-Eisler et al., PNAS 90(24): 11498-502, 1993; Guzman et al., Circulation 88(6): 2838-48, 1993; Guzman et al., Cir. Res. 73(6): 1202-1207, 1993; Zabner et al., Cell 75(2): 207-216, 1993; Li et al., Hum Gene Ther. 4(4): 403-409, 1993; Caillaud et al., Eur. J. Neurosci. 5(10): 1287-1291, 1993), adeno-associated type 1 (“AAV-1”) or adeno-associated type 2 (“AAV-2”) vectors (see WO 95/13365; Flotte et al., PNAS 90(22): 10613-10617, 1993), hepatitis delta vectors, live, attenuated delta viruses and herpes viral vectors (e.g., U.S. Pat. No. 5,288,641), as well as vectors which are disclosed within U.S. Pat. No. 5,166,320. Other representative vectors include retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218). Methods of using such vectors in gene therapy are well known in the art, see, for example, Larrick, J. W and Burck, K. L., Gene Therapy: Application of Molecular Biology, Elsevier Science Publishing Co., Inc., New York, N.Y., 1991; and Kreigler, M., Gene Transfer and Expression. A Laboratory Manual, W. H. Freeman and Company, New York, 1990.

Gene-delivery vehicles may be introduced into a host cell utilizing a vehicle, or by various physical methods. Representative examples of such methods include transformation using calcium phosphate precipitation (Dubensky et al., PNAS 81: 7529-7533, 1984), direct microinjection of such nucleic acid molecules into intact target cells (Acsadi et al., Nature 352: 815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89: 6094, 1990), lipofection (Feigner et al., Proc. Natl. Acad. Sci. USA 84: 7413-7417, 1989), microprojectile bombardment (Williams et al., PNAS 88: 2726-2730, 1991), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther. 29: 69, 1985; and Friedmann et al., Science 244: 1275, 1989), and DNA ligand (Wu et al, J of Biol. Chem. 264: 16985-16987, 1989), as well as psoralen inactivated viruses such as Sendai or Adenovirus.

Serum from mice immunized with gene delivery vehicles containing multivalent S. pneumoniae protein genes are assayed for total antibody titer by ELISA using native M proteins as the antigen. Serum opsonic antibodies are assayed as described above. Protective efficacy of DNA S. pneumoniae protein vaccines is determined by direct mouse protection tests using the serotypes of group A streptococci represented in the vaccine.

The invention also concerns human antibodies that specifically bind to selected S. pneumoniae protein antigens, wherein said human antibody is produced by non-human animal that comprises the human immune system. The non-human animal can be a mouse, for instance the SCID/SCID mouse of present invention.

The invention also provides a human antibody that specifically bind to a selected S. pneumoniae antigen of present invention that is a Fab fragment (F(ab)2dimer into an Fab′monomer) or single chain antibodies, including single chain Fv (sFv or scFv) antibodies. In various embodiments, the antibody-immunostimulant chimeric moieties in the compositions of the invention comprise an antibody fragment, or an Fab domain, an Fab′ domain, an F(ab′)2 domain, an F(ab)2domain, an scFv domain, IgG, IgA, IgE, IgM, IgD, IgG1, IgG2, IgG3.

The invention provides a polyvalent complex comprising at least two human antibodies each of which specifically binds to the selected S. pneumoniae antigen of present invention. The two different antibodies can be linked to each other covalently or non-covalently.

The invention provides a nucleic acid encoding a heavy chain of a human antibody. The nucleic acid can comprise a nucleotide sequence of antibodies which recognizes the amino acid (AA) sequences or fragments thereof set forth in any of the tables II to XI or of substantially identical AA sequences.

The invention further provides a pharmaceutical composition comprising a human antibody that specifically binds to the selected S. pneumoniae antigen of present invention and a pharmaceutically acceptable carrier. The antigen is a S. pneumoniae antigen of the group consisting of of zmpB, GroEL, ABC transporter (spermidine) or a S. pneumoniae antigen of the group consisting of ABC transporters (maltose/maltodextrin, SP_{—}1683), PTS system IIA component, pyruvate kinase, proteins SP_{—}1290 and protein SP_{—}0562. The pharmaceutical composition can further comprise an agent effective to induce an immune response against a target antigen. Also provided are chemotherapeutic agents. In addition, antibodies to immunosuppressive molecules are also provided.

The invention provides a method for inducing, augmenting or prolonging an immune response to selected protein antigens of S. pneumoniae in a patient, comprising administering to the patient an effective dosage of a human antibody that specifically binds to selected S. pneumoniae protein antigens as provided hereinbefore. The antigen is in particular a S. pneumoniae antigen of the group consisting of SP_{—}0562, SP_{—}1683 and SP_{—}1386.

This method can further comprise administering the S. pneumoniae antigens as provided hereinbefore, in particular selected from the group consisting of SP_{—}0562, SP_{—}1683 and SP_{—}1386, or a fragment or an analog thereof, to the patient, whereby this protein antigen or fragment thereof in combination with the human antibody induces, augments or prolongs the immune response. This method can further comprise administering an immunemodulator such as a cytokine (as described further in this application) to the patient.

The present invention further provides isolated or recombinant human antibodies and human monoclonal antibodies which specifically bind to selected S. pneumoniae antigens antigens as provided hereinbefore, in particular selected from the group consisting of SP_{—}0562, SP_{—}1683 and SP_{—}1386, or a fragment or an analog thereof, as well as compositions containing one or a combination of such antibodies.

Accordingly, the human antibodies and the human monoclonal antibodies of the invention can be used as diagnostic or therapeutic agents in vivo and in vitro. The human antibodies of the invention can encompass various antibody isotypes, or mixtures thereof, such as IgG1, IgG2, IgG3, IgG4, IgM, IgA1, IgA2, IgAsec, IgD, and IgE. Typically, they include IgG1 (e.g., IgG1k) and IgM isotypes. The human antibodies can be full-length (e.g., an IgG1 or IgG4 antibody) or can include only an antigen-binding portion (e.g., a Fab, F(ab)2, Fv or a single chain Fv fragment). Some human antibodies are recombinant human sequence antibodies. Some human antibodies are produced by a hybridoma which includes a B cell obtained from a transgenic non-human animal or the humanized SCID/SCID mouse of present invention, having a genome comprising a human heavy chain transgene and a human light chain transgene. The hybridoma can be made by, e.g., fusing the B cell to an immortalized cell.

Some human antibodies of the present invention can be characterized by one or more of the following properties: a) specificity for the a selected S. pneumoniae antigen of present invention; b) a binding affinity to the a selected S. pneumoniae antigen of present invention with an equilibrium association constant (Ka) of at least about 107 M-1, or about 109 M-1, or about 1010 M-1 to 1011 M-1 or higher; c) a kinetic association constant (ka) of at least about 103, about 104, or about 105 m-1s-1; and/or, d) a kinetic disassociation constant (kd) of at least about 103, about 104, or about 105 m-1 s-1.

In another aspect, the invention provides nucleic acid molecules encoding the human antibodies, or antigen-binding portions, of the invention. Accordingly, recombinant expression vectors that include the antibody-encoding nucleic acids of the invention, and host cells transfected with such vectors, are also encompassed by the invention, as are methods of making the antibodies of the invention by culturing these host cells.

In yet another aspect, the invention provides isolated human B-cells from a transgenic non-human animal or a humanized SCID mice, which are capable of expressing various isotypes (e.g., IgG, IgA and/or IgM) of human monoclonal antibodies that specifically bind to the a selected S. pneumoniae antigen of present invention for instance the S. pneumoniae antigens as provided hereinbefore, in particular selected from the group consisting of SP_{—}0562, SP_{—}1683 and SP_{—}1386 or a fragment or an analog thereof.

The isolated B cells can be obtained from a humanized SCID/SCID mouse. SCID mice were immunized with inactivated S. pneumoniae or with the isolated S. pneumoniae proteins of the group consisting of antigens as provided hereinbefore, in particular selected from the group consisting of SP_{—}0562, SP_{—}1683 and SP_{—}1386, or a fragment or an analog thereof in purified or enriched preparation of the selected protein antigen or mixtures thereof of present invention (or antigenic fragment thereof) and/or cells expressing the a selected S. pneumoniae antigen of present invention. The isolated B-cells can be immortalized to provide a source (e.g., a hybridoma) of human monoclonal antibodies to the selected S. pneumoniae antigen of present invention.

Accordingly, the present invention also provides a hybridoma capable of producing human monoclonal antibodies that specifically bind to the a selected S. pneumoniae antigens as provided hereinbefore, in particular selected from the group consisting of SP_{—}0562, SP_{—}1683 and SP_{—}1386, or a fragment or an analog thereof. The hybridoma can also include a B cell obtained from a transgenic non-human animal, e.g., the humanized SCID/SCID mouse of present invention or a transgenic mouse for instance “HuMAb-Mouse”, having a genome comprising a human heavy chain transgene and a human light chain transgene fused to an immortalized cell.

Such transgenic non-human animal can be immunized with a purified or enriched preparation of the selected S. pneumoniae antigens of present invention and/or cells expressing

The human monoclonal antibodies of the invention against the selected protein antigens of present invention, or antigen binding portions thereof (e.g., Fab), can be derivatized or linked to another functional molecule, e.g., another peptide or protein (e.g., an Fab′ fragment). For example, an antibody or antigen-binding portion of the invention can be functionally linked (e.g., by chemical coupling, genetic fusion, noncovalent association or otherwise) to one or more other molecular entities.

In another aspect, the present invention provides compositions, e.g., pharmaceutical and diagnostic compositions, comprising a pharmaceutically acceptable carrier and at least one human monoclonal antibody of the invention, or an antigen-binding portion thereof, which specifically binds to the a selected S. pneumoniae antigen of present invention. Some compositions comprise a combination of the human antibodies or antigen-binding portions thereof, preferably each of which binds to a distinct epitope. Compositions, e.g., pharmaceutical compositions, comprising a combination of at least one human antibodies or at least one human monoclonal antibody of the invention, or antigen-binding portions thereof, and at least one bispecific or multispecific molecule of the invention, are also within the scope of the invention.

Formulation and Administration : For therapeutic use, vaccinating agents can be administered to a patient by a variety of routes, including for example, by intramuscular, subcutaneous, and mucosal routes. The vaccinating agent may be administered as a single dosage, or in multiple units over an extended period of time. Within preferred embodiments, the vaccinating agent is administered to a human at a concentration of 50-300 ug per single site intramuscular injection. Several injections can be given (e.g., three or four) at least one month apart in order to further increase vaccine efficacy.

Typically, the vaccinating agent will be administered in the form of a pharmaceutical composition comprising purified polypeptide in conjunction with physiologically acceptable carriers, excipients or diluents. Such carriers will be nontoxic to patients at the dosages and concentrations employed. Ordinarily, the preparation of such compositions entails combining the vaccinating agent with buffers, antioxidants such as ascorbic acid, low molecular weight (less than about 10 residues) polypeptides, proteins, amino acids, carbohydrates including glucose, sucrose or dextrans, chelating agents such as EDTA, glutathione and other stabilizers and excipients. Neutral buffered saline or saline mixed with conspecific serum albumin are exemplary appropriate diluents.

Within preferred embodiments of the invention, the vaccinating agent is combined with an adjuvant, such as, for example, Freund's adjuvant, alum and the like. Within certain further embodiments, the vaccinating composition may further comprise an adjuvant, such as an immunomodulatory cofactor such as (but not limited to) IL-4, IL-10, γ-IFN, or IL-2, IL-12 or IL-15 or such the immunostimulant such as e.g., cytokines, chemokines, interleukins, interferons, C-X-C chemokines, C-C family chemokines, C chemokines, CX3C chemokines, super antigens, growth factors, IL-1, IL-2, IL-4, IL-6, M-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and functional fragments thereof of any of the above.

or such adjuvant may be antibody based. For instance a chimeric moiety comprising an antibody attached to a S. pneumoniae related antigen of present invention and the chimeric moiety comprising an adjuvant of this antigen. This can further potentiate an effective immune response (humoral and/or cellular) against the S. pneumoniae antigens in a human for efficient prophylactically or therapeutically treating a S. pneuminiae related disorder. By administering the antibody-immunostimulant chimeric moiety that comprises an adjuvant of the disease related antigen arising from the subject, a disease state within the subject, or a disease related organism within the subject, where the administration elicits an immune response within the subject against the disease related antigen. Where the antibody and/or the immunostimulatory component of the chimeric moiety are both single chain proteins and relatively short (i.e., less than about 50 amino acids) they can be synthesized using standard chemical peptide synthesis techniques. Where both componets are relatively short the chimeric moiety can be synthesized as a single contiguous polypeptide. Alternatively the antibody and the effector molecule may be synthesized separately and then fused by condensation of the amino terminus of one molecule with the carboxyl terminus of the other molecule thereby forming a peptide bond. Alternatively, the antibody and immunostimulatory molecule(s) may each be condensed with one end of a peptide spacer molecule thereby forming a contiguous fusion protein. Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Meffifield, et al. J. Am. Chem. Soc., 85: 2149-2156 (1963), and Stewart et al., Solid Phase Peptide Synthesis, 2nd ed. Pierce Chem. Co., Rockford, Ill. (1984). In certain embodiments, chimeric fusion proteins of the present invention are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein. DNA encoding the fusion proteins of this invention can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences can be obtained by the ligation of shorter sequences. Alternatively, subsequences can be cloned and the appropriate subsequences cleaved using appropriate restriction enzymes. The fragments can then be ligated to produce the desired DNA sequence. Thus, for example DNA encoding fusion proteins of the present invention can be cloned using DNA amplification methods such as polymerase chain reaction (PCR). Thus, for example, the nucleic acid encoding a particular antibody is PCR amplified, using a sense primer containing the restriction site for NdeI and an antisense primer containing the restriction site for HindIII. This produces a nucleic acid encoding the antibody sequence and having terminal restriction sites. A nucleic acid encoding the immunostimulatory molecule(s) can similarly be produces. Ligation of the antibody and immunostimulatory molecule sequences and insertion into a vector produces a vector encoding the antibody joined to the immunostimulatory molecule. While the components comprising the chimeric moiety can be joined directly together, in certain embodiments, the components can be separated by a peptide spacer consisting of one or more amino acids. Generally the spacer will have no specific biological activity other than to join the proteins or to preserve some minimum distance or other spatial relationship between them. However, the constituent amino acids of the spacer can be selected to influence some property of the molecule such as the folding, net charge, or hydrophobicity. The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences. The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes. Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically. One of skill in the art would recognize that after chemical synthesis, biological expression, or purification, the fusion protein can possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270). One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

Previously, antibody-(IL-2) fusion proteins have been the best characterized and most broadly used in successful anti-tumor experiments using animal models (see, e.g., Penichet and Morrison, 2001, “Antibody-cytokine fusion proteins for the therapy of cancer” J Immunol Met 248:91-101).

Numerous studies have explored various combinations of antibodies and, e.g., IL-2, as direct targeting agents of tumor cells. For example, a tumor specific antibody-(IL-2) fusion protein has been developed which comprised a human IgG3 specific for the idiotype (Id) of the Ig expressed on the surface of the B cell lymphoma 38C13 with human IL-2 fused at the end of the CH 3 domain. See, Penichet et al., 1998 “An IgG3-IL-2 fusion protein recognizing a murine B cell lymphoma exhibits effective tumor imaging and antitumor activity” J Interferon Cytokine Res 18:597-607. That antibody fusion protein, IgG3-CH3-(IL-2), was expressed in Sp2/0 and was properly assembled and secreted. Anti-Id IgG3-CH3-(IL-2) has a half-life in mice of approximately 8 hours, which is 17-fold longer than the half-life reported for IL-2 (i.e., when not fused to another domain), and it showed a better localization of subcutaneous tumors in mice than the anti-Id IgG3 by itself. Most importantly, the anti-Id IgG3-CH3-(IL-2) showed enhanced anti-tumor activity compared to the combination of antibody and IL-2 administered together. Again, see, Penichet et al., 1998, supra. Additionally, a chimeric anti-Id IgG1-(IL-2) fusion protein (chS5A8-IL-2) expressed in P3X63Ag8.653 has shown more effectiveness in the in vivo eradication of the 38013 tumor than the combination of the anti-Id antibody and IL-2 or an antibody-(IL-2) fusion protein with an irrelevant specificity. See, Liu et al., 1998 “Treatment of B-cell lymphoma with chimeric IgG and single-chain Fv antibody-interleukin-2 fusion proteins” Blood 92:21030-12. Another example of previous antibody fusion proteins is in cancer treatment and involved chimeric anti-GD2 IgG1-(IL-2) fusion protein (ch14.18-IL-2) produced in Sp2/0 cells. See, Becker et al., 1996 “T cell-mediated eradication of murine metastatic melanoma induced by targeted interleukin 2 therapy” J Exp Med 183:2361-6; Becker et al., 1996 “An antibody-interleukin 2 fusion protein overcomes tumor heterogeneity by induction of a cellular immune response” Proc Natl Acad Sci USA 93:7826-31; and Becker et al., 1996 “Long-lived and transferable tumor immunity in mice after targeted interleukin-2 therapy” J Clin Invest 98:2801-4. The ch14.18-IL-2 treatment of mice which had pulmonary and hepatic metastases, as well as subcutaneous GD2 expressing B16 melanoma, resulted in a specific and strong anti-tumor activity. This anti-tumor activity was significant compared to antibody (ch14.18) and IL-2 or irrelevant antibody-(IL-2) fusion proteins and resulted in the complete eradication of the tumor in a number of animals. See, Becker references, supra. Similar results have been obtained in mice bearing CT26-KSA hepatic and pulmonary metastases and treated with a humanized anti-KSA antibody-IL-2 fusion protein (huKS1/4-IL-2) produced in NSO. See, Xiang et al., 1997 “Elimination of established murine colon carcinoma metastases by antibody-interleukin 2 fusion protein therapy” Cancer Res 57:4948-55 and Xiang et al., 1999 “T cell memory against colon carcinoma is long-lived in the absence of antigen” J Immunol 163:3676-83. Other examples of antibody fusion molecules include a chimeric anti-human MHC class II IgG1 fused to GMCSF (chCLL-1/GMCSF) expressed in NSO (see, Homick et al., 1997 “Chimeric CLL-1 antibody fusion proteins containing granulocyte-macrophage colony-stimulating factor or interleukin-2 with specificity for B-cell malignancies exhibit enhanced effector functions while retaining tumor targeting properties” Blood 89:4437-47) and a humanized anti-HER2/neu IgG3 fused to IL-12 (see, Peng et al., 1999, “A single-chain IL-12 IgG3 antibody fusion protein retains antibody specificity and IL-12 bioactivity and demonstrates antitumor activity” J Immunol 163:250-8), IL-2 (see, Penichet et al., 2001, “A recombinant IgG3-(IL-2) fusion protein for the treatment of human HER2/neu expressing tumors” Human Antibodies 10:43-49) and GMCSF expressed in P3X63Ag8.653 (see, Dela Cruz et al., 2000, “Recombinant anti-human HER2/neu IgG3-(GMCSF) fusion protein retains antigen specificity, cytokine function and demonstrates anti-tumor activity” J Immunol 165:5112-21). It is important to note that the antibody-cytokine fusion proteins containing IL-2, IL-12, or GMCSF, etc. have been used as direct antitumor agents which directly targeted tumors in animal models. The antibody fusion proteins bound to antigens on tumor surfaces, thus increasing the local concentration of, e.g., Il-2, etc. around the tumor. The increased, e.g., IL-2, thus lead to anti-tumor activity in some cases. See, e.g., Penichet, et al. 2001, supra. Some work describes linking antigens to IL-2 via an IgG3-(IL-2) fusion protein with affinity for a convenient hapten antigen, dansyl (DNS). See, Harvill et al., 1996 “In vivo properties of an IgG3-Il-2 fusion protein. A general strategy for immune potentiation” J Immunol 147:3165-70. The antigen used in this work was highly artificial (bovine serum albumin) rather than a disease-related antigen. Using hapten-conjugated-bovine serum albumin (DNS-BSA) as a model antigen the inventors showed an antibody response elicited by anti-DNS-IgG3-(IL-2)-bound DNS-BSA injected into mice increased over that of DNS-BSA-Sepharose, anti-DNS-IgG3-bound DNS-BSA, or a non-specific IgG3-(IL-2)-bound DNS-BSA. The binding of the antibody-(IL-2) fusion protein to the antigen (non-covalent physical linkage) was shown to enhance the immune response (see, Harvill et al., 1996, supra invention provides these and other approaches and methods in treatment. Such antibody-immunostimulant chimeric moieties (e.g., protein fusions) can be as adjuvants for antigenic protein vaccinations and methods of prophylactically and/or therapeutically treating a pneumococcal disorder in a subject. A suitable composition is one that comprises an antibody-immunostimulant chimera (chimeric moiety) where the chimera is capable of acting as an effective adjuvant of antigens of present invention whereby the antibody-immunostimulant chimera preferably has antibody specificity against these selected antigens. Such composition may also include the Streptococcus pneumoniae antigens the disease related antigen. Certain preferred chimeric moieties comprise an antibody directed against an antigen (e.g. a disease-producing antigen) attached (directly or through one or more linkers) to one or more immunostimulatory molecules (e.g., to two immunostimulatory molecules, three immunostimulatory molecules, four or more immunostimulatory molecules). In certain embodiments, where two or more immunostimulatory molecules are present, the immunostimulatory molecules are different species. Certain embodiments also contemplate the use of two or more antibodies in conjunction, with one, two, or more immunostimulatory molecules. In certain embodiments the chimeric moiety is a fusion protein where the antibody is coupled directly or thorugh a peptide linker to one or more immunostimulants (immunostimulatory molecules). The immunostimulant domain(s) of the chimeric moieties (e.g., fusion proteins) in these compositions can, in certain embodiments comprise a cytokine (or a sequence or subsequence thereof), a chemokine (or a sequence or subsequence thereof), or an immunostimulant other than a chemokine or cytokine. Examples of such immunostimulant domains include, but are not limited to cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, M-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof of any of the above. The antibody domain/component of the chimeric moieties in the compositions of the invention optionally includes, but is not limited to, an antibody specific for the Streptococcus pneumoniae proteins of the group consisting of zmpB, GroEL, and ABC transporter (spermidine). The antibody domain/component of the chimeric moieties in the compositions of the invention may includes, but is not limited to, an antibody specific for the Streptococcus pneumoniae antigens as provided hereinbefore, in particular selected from the group consisting of SP_{—}0562, SP_{—}1683 and SP_{—}1386. In various embodiments, the antibody-immunostimulant chimeric moieties in the compositions of the invention comprise an antibody fragment, or an Fab domain, an Fab′ domain, an F(ab′)2 domain, an F(ab)2domain, an scFv domain, IgG, IgA, IgE, IgM, IgD, IgGI, IgG2, IgG3. Such chimeric moiety may comprise a cytokine (or a sequence or subsequence thereof), a chemokine (or a sequence or subsequence thereof), or an immunostimulant other than a chemokine or cytokine. In other embodiments of this aspect, the method uses fusion proteins comprising an immunostimulant domain such as (but not limited to), e.g., cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof of any of the above.

Effective Dosages: Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician can start doses of the compounds of the invention employed in the pharmaceutical composition at levels lower than that required to achieve the desired therapeutic effect and gradually increase the dosage until the desired effect is achieved. In general, a suitable daily dose of compositions of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of therapeutic compositions can be administered as two; three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Effective doses of the compositions of the present invention, for the treatment of immune-related conditions and diseases described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy.

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody to S pneumoniae protein antigen in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

Some human antibodies and human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes may comprise one or more moieties which are selectively transported into specific cells or organs, thus enhance targeted drug delivery (See, e.g., V. V. Ranade (1989) J. Clin. Pharmacol. 29:685). Exemplary targeting moieties include folate or biotin (See, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); See also K. Keinanen; M. L. Laukkanen (1994) FEBS Lett. 346:123; J. J. Killion; I. J. Fidler (1994) Immunomethods 4:273. In some methods, the therapeutic compounds of the invention are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

For therapeutic applications, the pharmaceutical compositions are administered to a patient suffering from established disease in an amount sufficient to arrest or inhibit further development or reverse or eliminate, the disease, its symptoms or biochemical markers. For prophylactic applications, the pharmaceutical compositions are administered to a patient susceptible or at risk of a disease in an amount sufficient to delay, inhibit or prevent development of the disease, its symptoms and biochemical markers. An amount adequate to accomplish this is defined as a “therapeutically-” or “prophylactically-effective dose.” Dosage depends on the disease being treated, the subject's size, the severity of the subject's symptoms, and the particular composition or route of ‘administration selected. Specifically, in treatment of tumors, a “therapeutically effective dosage” can inhibit tumor growth by at least about 20%, or at least about 40%, or at least about 60%, or at least about 80% relative to untreated subjects. The ability of a compound to inhibit cancer can be evaluated in an animal model system predictive of efficacy in human tumors. Alternatively, this property of a composition can be evaluated by examining the ability of the compound to inhibit by conventional assays in vitro. A therapeutically effective amount of a therapeutic compound can decrease tumor size, or otherwise ameliorate symptoms in a subject.

The composition should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

Routes of Administration: Pharmaceutical compositions of the invention also can be administered in combination therapy, i.e., combined with other agents.

Pharmaceutically acceptable carriers includes solvents, dispersion media, coatings, antibacterial and antifungal agents, isotonic and absorption delaying agents, and the like that are physiologically compatible. The carrier can be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (See, e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono-and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, 1978. Pharmaceutical compositions are preferably manufactured under GMP conditions.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred embodiment, a therapeutic composition of the invention can be administered with a needleless hypodermic injection device,'such as the devices disclosed in, e.g., U.S. Pat. Nos. 5,399,163, 5,383,851, 5,312,335, 5,064,413, 4,941, 880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

Formulation: For the therapeutic compositions, formulations of the present invention include those suitable for oral, nasal, topical (including buccal and sublingual), rectal, vaginal and/or parenteral administration. The formulations can conveniently be presented in unit dosage form and may be prepared by any methods known in the art of pharmacy. The amount of active ingredient which can be combined with a carrier material to produce a single dosage form vary depending upon the subject being treated, and the particular mode of administration. The amount of active ingredient which can be combined with a carrier material to produce a single dosage form generally be that amount of the composition which produces a therapeutic effect. Generally, out of one hundred percent, this amount range from about 0.01 percent to about ninety-nine percent of active ingredient, from about 0.1 percent to about 70 percent, or from about 1 percent to about 30 percent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

The pharmaceutical compositions are generally formulated as sterile, substantially isotonic and in full compliance with all Good Manufacturing Practice (GMP) regulations of the U.S. Food and Drug Administration.

Delivery via antigen presenting cells: An aspect of present invention is delivery the S. pneumoniae proteins ex vivo to antigen-presenting cell by targeting the preselected antigen to an endocytic receptor on the antigen-presenting cell. A non-limiting but preferred antigen-presenting cell is a dendritic cell (DC). using antigen-presenting cells isolated from the patient, after which the cells may be optionally isolated and returned to the patient. Both, the antigen exposure and DC maturation may be carried out ex vivo . Various routes of delivery are embraced herein, including but not limited to parenteral or transmucosal delivery. Parenteral includes but is not limited to, intra-arterial, intramuscular, intradermal, subcutaneous, intraperitoneal, intraventricular, and intracranial administration. Pulmonary, intraintestinal, and delivery across the blood brain barrier are also embraced herein. Dendritic cells (DCs) are uniquely potent inducers of primary immune responses in vitro and in vivo (J. Banchereau, R. M. Steinman, Nature 392, 245-52 (1998); C. Thery, S. Amigorena, Curr. Opin. Immunol. 13, 45-51. (2001)). In tissue culture experiments, DCs are typically two orders of magnitude more effective as antigen presenting cells (APCs) than B cells or macrophages (K. Inaba, R. M. Steinman, W. C. Van Voorhis, S. Muramatsu, Proc Natl Acad Sci USA 80, 6041-5 (1983); R. M. Steinman, B. Gutchinov, M. D. Witmer, M. C. Nussenzweig, J Exp Med 157, 613-27 (1983)). In addition, purified, antigen-bearing DCs injected into mice or humans migrate to lymphoid tissues and efficiently induce specific immune responses (M. V. Dhodapkar, et al., J Clin Invest 104, 173-80 (1999); K. Inaba, J. P. Metlay, M. T. Crowley, R. M. Steinman, J Exp Med 172, 631-40 (1990); R. I. Lechler, J. R. Batchelor, J Exp Med 155, 31-41 (1982)). Likewise, DCs migrate from peripheral tissues to lymphoid organs during contact allergy (S. E. Macatonia, S. C. Knight, A. J. Edwards, S. Griffiths, P. Fryer, J Exp Med 166, 1654-67 (1987); A. M. Moodycliffe, et al., J Exp Med 191, 2011-20 (2000)) and transplantation (C. P. Larsen, P. J. Morris, J. M. Austyn, J Exp Med 171, 307-14 (1990)), two of the most powerful, known stimuli of T cell immunity in vivo. Based on these and similar experiments, it has been proposed that the principal function of DCs is to initiate T cell mediated immunity (J. Banchereau, R. M. Steinman, Nature 392, 245-52 (1998)).

Microbial surface peptide polypeptide or protein antigens sceening system: A further aspect of present invention is a method to screen on microbial organisms for surface peptide, surface polypeptide or surface protein antigens which are immunogenic in human, induce an immune response or induce an immunological memory if the microbial organism invades the human body that is characterised in that 1) non viable microbial organism are delivered to severe combined immunodeficient (SCID/SCID) mice that received a natural killer (NK) cell depleting treatment and that has been treated by human PBMC, 2) that the immune response is evaluated by detecting antibodies against known human immunogens for that microbial organism and 3) that immunogenic surface peptide, surface polypeptide or surface protein antigens are identified. This method of sceening for microbial surface peptide polypeptide or protein antigens which are immunogenic in human, induce an immune response or induce an immunological memory if the microbial organism invades the human body is characterised in that it comprises the steps of 1) delivering to severe combined immunodeficient (SCID/SCID) mice a natural killer (NK) cell depleting compound, 2) treating the severe combined immunodeficient (SCID/SCID) mice by human PBMC, 3) delivering non viable microbial organism in the severe combined immunodeficient (SCID/SCID) mice, 4) evaluating the immune response by detecting antibodies against known human immunogens for that microbial organism and 5) identifying the immunogenic surface peptide polypeptide or protein antigens. In this mthod the severe combined immunodeficient (SCID/SCID) mice can be of the group of consisting of a C.B-17/Icr scid/scid mice, CB17-scid/scid (SCID, H-2d) mice and C57BL/6 (B6), BALB/c, scid/scid (SCID) mice and their natural killer (NK) cell can be depleted by a CD122 anatogonist for instance a ligand of the IL-2 receptor beta-chain that inhibits the CD122 (interleukin-2 receptor β, IL-2Rβ) receptor which can be an antibody or antigen-binding fragment thereof directed against the mouse IL-2 receptor beta-chain, preferably the mouse IL-2 receptor beta-chain is TMβ1, In this method the non viable microbial organism can be delivered intraperitoneally (i.p.), also the ligand of the IL-2 receptor beta-chain that inhibits the CD122 (interleukin-2 receptor 13, IL-2R(3) receptor can be delivered by intraperitoneal (i.p.) injection a²nd also the human PBMC can be intraperitoneally (i.p.) delivered. Optimal results are obtained when the SCID/SCID mice received the natural killer cell depleting treatment by a ligand of the IL-2 receptor beta-chain that inhibits the CD122 (interleukin-2 receptor β, IL-2Rβ) receptor before transferring human PBMC to said mice at least 12 hours before transferring human PBMC to said mice, for instance one day on forehand.

The immune response of the immunodeficient (SCID/SCID) mice is determined by detecting antibodies against the microbial organisms. Human monoclonal antibodies, having the desired specificity and the characteristics, are for instance produced by transformation of B lymphocytes obtained from peripheral blood of the SCID/SCID mice which have been injected with the killed or non viable microbial cells according to the method of present invention.

A specific protocol is for instance the production of a human anti-zmpC (S. pneumoniae) monoclonal antibody. Peripheral vein blood is collected from S. pneumoniae treatment Scid/scid mouse according the protocol of present invention with inhibitor. Peripheral blood mononuclear cells (PBMC) are prepared by Ficoll-Hypaque density centrifugation using standard methods. All cell cultures are carried out in Dulbecco's MEM/Nutrient Mix P12 (Life Technologies) supplemented with 10% IgG-free horse serum, 1.5 g/l glucose, 4 mM L-glutamine, 1% Caryoser and 80 mg/L Geomycin. PBMC are immortalised as follows. 10⁷ PBMC are resuspended in 2 ml culture medium and incubated for 2 h at 37° C. with 200 μl Epstein-Barr virus (EBV) supernatant (B95-8 strain). Cells are then seeded at 300 to 24,000 cells/well in 96-well microtiter plates (Nunc, Roskilde, Denmark) containing 3T6-TRAP cells treated with mitomycin C (50 μg/mi) for lh at 37° C., and seeded in culture wells the day prior to EBV infection of PBMC. The 3T6 cell line have been stably transfected with an expression vector for human CD4O ligand (3T6-TRAP). One hundred and fifty μl of culture supernatant are placed every week by fresh culture medium. After 4 to 8 weeks, depending on growth rate in individual wells, culture supernatants are tested in ELISA for the presence of anti-zmpC antibodies. Positive cell lines are transferred to 24-well plates, and immediately cloned at 60 cells per 96-well plate without feeder cells.

Thus, antibodies towards zmpC are identified by reacting the supernatant with polystyrene plates coated with zmpC. The binding of specific antibodies is detected by addition of an anti-human IgG reagent coupled to an enzyme. Addition of an enzyme substrate that is converted to a coloured compound in the presence of the enzyme allows the detection of specific antibodies. Such methods referred to as Enzyme-Linked Immuno-Sorbent Assays (ELISA) are well known by those skilled in the art. Detailed description can be found in Current Protocols in Lmmunology, Chapter 2, John Wiley & Sons, mc, 1994.

B cells producing anti- zmpC antibodies are then expanded and cloned by limiting dilution. Methods to carry out cloning are described for instance in Current Protocols in Immunology, Chapter 2, John Wiley & Sons, mc, 1994.

Antibodies with sufficient binding avidity for zmpC and which inhibit virulent function of growth of S. pneumoniae are then produced in bulk culture and purified by affinity chromatography using methods well known by those skilled in the art. This method can be used to produce monoclonal human antibodies against other microbal surface (poly)peptide or protein antigens.

As already mentioned hereinbefore, alternatively, the antibodies can also be generated and selected using various phage display methods known in the art. In the antibody libraries as used in said phage display methods, the antibody chains can be displayed in single or double chain form. Single chain antibody libraries can comprise the heavy or light chain of an antibody alone or the variable domain thereof. However, more typically, the members of single-chain antibody libraries are formed from a fusion of heavy and light chain variable domains separated by a peptide spacer within a single contiguous protein. See e.g., Ladner, et al., WO 88/06630; McCafferty, et al., WO 92/01047. Double-chain antibodies are formed by noncovalent association of heavy and light chains or binding fragments thereof. Double chain antibodies can also form by association of two single chain antibodies, each single chain antibody comprising a heavy chain variable domain, a linker and a light chain variable domain. In such antibodies, known as diabodies, the heavy chain of one single-chain antibody binds to the light chain of the other and vice versa, thus forming two identical antigen binding sites (see Hollinger et al., Proc. Natl. Acad. Sci. USA 90, 6444-6448 (1993) and Carter & Merchan, Curr. Op. Biotech. 8, 449-454 (1997). Thus, phage displaying single chain antibodies can form diabodies by association of two single chain antibodies as a diabody.

The diversity of antibody libraries can arise from obtaining antibody-encoding sequences from a natural source, such as a nonclonal population of immunized or unimmunized B cells. Alternatively, or additionally, diversity can be introduced by artificial mutagenesis of nucleic acids encoding antibody chains before or after introduction into a display vector. Such mutagenesis can occur in the course of PCR or can be induced before or after PCR.

Nucleic acids encoding antibody chains to be displayed optionally flanked by spacers are inserted into the genome of a display package as discussed above by standard recombinant DNA techniques (see generally, Sambrook, et al., Molecular Cloning, A Laboratory Manual, 2d ed., Cold Spring Harbor Laboratory Press, Cold Spring Harbor, N.Y., 1989, incorporated by reference herein). The nucleic acids are ultimately expressed as antibody chains (with or without spacer or framework residues). In phage, bacterial and spore vectors, antibody chains are fused to all or part of the an outer surface protein of the replicable package. Libraries often have sizes of about 103, 104, 105, 106, 107, 108 or more members.

Double-chain antibody display libraries represent a species of the display libraries discussed above. Production of such libraries is described by, e.g., Dower, U.S. Pat. Nos. 5,427,908; U.S. Pat. No. 5,580,717, Huse WO 92/06204; Huse, in Antibody Engineering, (Freeman 1992), Ch. 5; Kang, WO 92/18619; Winter, WO 92/20791; McCafferty, WO 92/01047; Hoogenboom WO 93/06213; Winter, et al., Annu. Rev. Immunol. 12:433-455 (1994); Hoogenboom, et al., Immunological Reviews 130:41-68 (1992); Soderlind, et al , Immunological Reviews 130:109-124 (1992). For example, in double-chain antibody phage display libraries, one antibody chain is fused to a phage coat protein, as is the case in single chain libraries. The partner antibody chain is complexed with the first antibody chain, but the partner is not directly linked to a phage coat protein. Either the heavy or light chain can be the chain fused to the coat protein. Whichever chain is not fused to the coat protein is the partner chain. This arrangement is typically achieved by incorporating nucleic acid segments encoding one antibody chain gene into either gIII or gVIII of a phage display vector to form a fusion protein comprising a signal sequence, an antibody chain, and a phage coat protein. Nucleic acid segments encoding the partner antibody chain can be inserted into the same vector as those encoding the first antibody chain. Optionally, heavy and light chains can be inserted into the same display vector linked to the same promoter and transcribed as a polycistronic message. Alternatively, nucleic acids encoding the partner antibody chain can be inserted into a separate vector (which may or may not be a phage vector). In this case, the two vectors are expressed in the same cell (see WO 92/20791). The sequences encoding the partner chain are inserted such that the partner chain is linked to a signal sequence, but is not fused to a phage coat protein. Both antibody chains are expressed and exported to the periplasm of the cell where they assemble and are incorporated into phage particles.

Typically, only the variable region of human light and heavy chains are cloned from the immunized SCID/SCID mouse. In such instances, the display vector can be designed to express heavy and light chain constant regions or fragments thereof in-frame with heavy and light chain variable regions expressed from inserted sequences. Typically, the constant regions are naturally occurring human constant regions; a few conservative substitutions can be tolerated but are not preferred. In a Fab fragment, the heavy chain constant region usually comprises a CH1 region, and optionally, part or all of a hinge region, and the light chain constant region is an intact light chain constant region, such as Cκ or Cλ. Choice of constant region isotype depends in part on whether complement-dependent cytotoxity is ultimately required. For example, human isotypes IgG1 and IgG4 support such cytotoxicity whereas IgG2 and IgG3 do not. Alternatively, the display vector can provide nonhuman constant regions. In such situations, typically, only the variable regions of antibody chains are subsequently subcloned from display vectors and human constant regions are provided by an expression vector in frame with inserted antibody sequences.

In a further variation, both constant and variable regions can be cloned from the immunized SCID/SCID mouse. For example, heavy chain variable regions can be cloned linked to the CH1 constant region and light chain variable regions linked to an intact light chain constant region for expression of Fab fragments. In this situation, display vectors need not encode constant regions.

Repertoires of antibody fragments have been constructed by combining amplified VH and VL sequences together in several ways. Light and heavy chains can be inserted into different vectors and the vectors combined in vitro (Hogrefe, et al., Gene 128:119-126 (1993)) or in vivo (Waterhouse, et al., Nucl. Acids. Res.: 2265-66 (1993)). Alternatively, the light and heavy chains can be cloned sequentially into the same vector (Barbas, et al., Proc. Natl. Acad. Sci. USA 88: 7987-82 (1991)) or assembled together by PCR and then inserted into a vector (Clackson, et al., Nature 352:624-28 (1991)). Repertoires of heavy chains can be also be combined with a single light chain or vice versa. Hoogenboom, et al., J. Mol. Biol. 227: 381-88 (1992).

Typically, segments encoding heavy and light antibody chains are subcloned from separate populations of heavy and light chains resulting in random association of a pair of heavy and light chains from the populations in each vector. Thus, modified vectors typically contain combinations of heavy and light chain variable region not found in naturally occurring antibodies. Some of these combinations typically survive the selection process and also exist in the polyclonal libraries described below.

Some exemplary vectors and procedures for cloning populations of heavy chain and light chain encoding sequences have been described by Huse, WO 92/06204. Diverse populations of sequences encoding Hc polypeptides are cloned into M131X30 and sequences encoding Lc polypeptides are cloned into M13IX11. The populations are inserted between the XhoI-SeeI or Stul restriction enzyme sites in M13IX30 and between the SacI-Xbal or EcoR V sites in M13IX11. Both vectors contain two pairs of MluI-HindIII restriction enzyme sites for joining together the Hc and Lc encoding sequences and their associated vector sequences. The two pairs are symmetrically orientated about the cloning site so that only the vector proteins containing the sequences to be expressed are exactly combined into a single vector.

This method of present invention to screen microbial organisms for surface peptide polypeptide or protein antigens which are immunogenic in human, induce an immune response or induce an immunological memory if the microbial organism invades the human body, is particularly suitable to identify new surface peptide polypeptide or protein antigens on human pathogenic microbial organisms such as microbial fungi, spirochetes, protozoa and bacteria. This method of present invention can further involves the methiod steps to isolate newly identified surface peptide polypeptide or protein antigens or the antibodies for furter use to manufacture a medicament of a diagnostic tool. The surface peptide polypeptide or protein antigens or antibodies produced by this process are part of present invention.

This method is particularly suitable to screen on infectious bacterial, fungal or protozoan microbials for surface peptide polypeptide or protein antigens which are immunogenic in human, induce an immune response or induce an immunological memory if the microbial organism invades the human body. Examples of bacterial infectious microbials include, but are not limited to, Actinomyces, Bacillus, Bacteroides, Bartonella, Bordetella, Borrelia, Brucella, Campylobacter, Capnocytophaga, Clostridium, Corynebacterium, Coxiella, Dermatophilus, Ehrlichia, Enterococcus, Escherichia, Francisella, Fusobacterium, Haemobartonella, Helicobacter, Klebsiella, L-form bacteria, Leptospira, Listeria, Mycobacteria, Mycoplasma, Neorickettsia, Nocardia, Pasteurella, Peptococcus, Peptostreptococcus, Proteus, Pseudomonas, Rickettsia, Rochalimaea, Salmonella, Shigella, Staphylococcus, Streptococcus, and Yersinia. Examples of fungal infectious microbials include, but are not limited to, Absidia, Acremonium, Altemaria, Aspergillus, Basidiobolus, Bipolaris, Blastomyces, Candida, Chlamydia, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Example of protozoan parasite infectious microbials include, but are not limited to, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium, Pneumocystis, Sarcocystis, Schistosoma, Theileria, Toxoplasma, and Trypanosoma.

This method is also particularly suitable to screen on infectious helminth parasite for surface peptide polypeptide or protein antigens which are immunogenic in human, induce an immune response or induce an immunological memory if the helminth parasite invades the human body. Examples of helminth parasite infectious agents include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca, Spirometra, Stephanofilaria, Strongyloides, Strongylus, Thelazia, Toxascaris, Toxocara, Trichinella, Trichostrongylus, Trichuris, Uncinaria, and Wuchereria.

The method for instance allows to screen on major pathogenic microbial fungi such as Candida spp (cause of candidiasis), Histoplasma capsulatum (cause of histoplasmosis) and Cryptococcus neoformans (cause of cryptococcosi), Pneumocystis jirovecii (cause of opportunistic pneumonia) and Tinea (cause of Dermatophytosis or Tinea ringworm) for surface peptide, polypeptide or protein antigens which are immunogenic in human, induce an immune response or induce an immunological memory if the microbial fungi invades the human body

The method also allows to screen on major pathogenic microbial protozoa such as Cryptosporidium (cause of cryptosporidiosis), Giardia lamblia (the cause of giardiasis), Plasmodium for instance Plasmodium falciparum (the cause of malaria) and Trypanosoma cruzi (the cause of chagas disease) for surface peptide, polypeptide or protein antigens which are immunogenic in human, induce an immune response or induce an immunological memory if the microbial protozoa invades the human body.

In yet another embodiment of present invention the method of present invention is used to screen on major pathogenic bacteria such as Escherichia coli (the cause of urinary tract infection, peritonitis, foodborne illness), Mycobacterium tuberculosis (the cause of tuberculosis), Bacillus anthracis (the cause of anthrax), Salmonella (the cause of foodborne illness), Staphylococcus aureus (the cause of toxic shock syndrome), Streptococcus pneumoniae (the cause of pneumonia), Streptococcus pyogenes (the cause of strep throat) and Helicobacter pylori (the cause of Stomach ulcers), Francisella tularensis (the cause of tularemia) for surface peptide, polypeptide or protein antigens which are immunogenic in human, induce an immune response or induce an immunological memory if the pathogenic bactery invades the human body.

The method of present is particularly suitable to screen on major pathogenic spirochetes bacteria of the genera Treponema, Borrelia, and Leptospira surface peptide, polypeptide or protein antigens which are immunogenic in human, induce an immune response or induce an immunological memory if the pathogenic spirochete invades the human body. The group of these spirochete bacteria have an outermost cell structure of a three-layered membrane called “outer sheath” or “outer cell envelope”corresponding to the “outer membrane” of gram-negative bacteria. In the genus Treponema common human human pathogenic Treponema are T. pallidum, T. pertenue, T. carateum, in the Borrelia genus Borrelia burgdorferi (the cause of Lyme disease) is well known and many other Borrelia spp are known to cause relapsing fever in humans and are transmitted by lice or ticks, while in the Leptospira genus host-associated leptospires can cause disease (leptospirosis) in humans by exposure to Leptospira which can be found in fresh water contaminated by animal urine. These microbials can be subjected to the method of present invention for the identification of new surface (poly)peptide or protein antigens.

The method even allows to screen on small parasitic worms such as the Schistosoma spp which cause schistosomiasis for surface peptide polypeptide or protein antigens which are immunogenic in human, induce an immune response or induce an immunological memory if the parasitic worms invades the human body. Such pathogenic Schistosoma spp are for instance Schistosoma haematobium, Schistosoma intercalatum, Schistosoma japonicum, Schistosoma mansoni or Schistosoma mekongi (the cause schistosomiasis), which can be subjected to the method of present invention for the identification of new surface (poly)peptide or protein antigens.

The method for instance allows to screen on major pathogenic microbial fungi for surface peptide, polypeptide or protein antigens which are immunogenic in human, induce an immune response or induce an immunological memory if the microbial fungi invades the human body whereby such microbial fungi can be of the group of pathogenic species consiting of Absidia spp., Acremonium spp., Actinomyces spp., Alternaria spp., Aspergillos spp., Aspergillus spp., Bipolaris spp., Bipolaris spp., Candida spp., Cladosporium spp., Curvularia spp., Erythematous Aspergillos spp., Exserohilum spp., Fusarium spp., Microsporum spp., Mucor spp., Paecilomyces spp., Rhizopus spp., Trichophyton spp. and Zygomycete spp.

In an other embodiment the method of present invention is used to screen on major pathogenic microbial fungi for surface peptide;:polypeptide or protein antigens which are immunogenic in human, induce an immune response or induce an immunological memory if the microbial fungi invades the human body whereby such microbial fungi can be of the group of consiting of Aspergillus flavus, Aspergillus fumigatus, Aspergillus fumigatus, Aspergillus terreus, Candida albicans, Candida albicans, Candida bantianum, Candida dubliniensis, Candida glabrata, Candida glabrata, Candida glabrata, Candida guilliermondii, Candida krusei, Candida krusei, Candida lusitaniae, Candida neoformans, Candida parapsilosis, Candida parapsilosis, Candida tropicales, Candida tropicales, Cladophialophora bantiana, Coccidioides immitis, Cryptococcus neoformans, Penicillium marneffei, Pseudallescheria boydii, Scedosporium apiospermum, Scedosporium prolificans, Scedosporium species, Tinea barbae, Tinea capitis, Tinea corporis, Tinea cruris, Tinea faciei, Tinea manuum, Tinea nigra, Tinea pedis, Tinea unguinum, and Tinea versicolor.

A particular embodiment is the method of present invention to screen on pathogenic bacteria for surface peptide, polypeptide or protein antigens, which are immunogenic in human, induce an immune response or induce an immunological memory if the pathogenic bactery invades the human body whereby the pathogenic bacteria are op the group consisting of Acinetobacter spp., Bacillus spp., Botulinum spp., Cholera spp., Entercoccus spp., Enterobacteriaceae spp. Streptococci spp, Klebsiella spp. and Meningococcal spp. or from the group consisting of Bordetella pertussis, Borrelia burgdorferi, Campylobacter jejuni, Chlamydia trachomatis, Clostridium difficile, Clostridium tetani, Corynebacterium diphtheria, Cryptococcus neoformans, Diphtheria tetanus, Enterococcus faecalis, Escherichia coli, Group A Streptococcal, Haemophilus influenzae (in particular Haemophilus influenzae type b (Hib)), Hib meningococcal, Klebsiella pneumoniae, Legionella pneumophila, Listeria monocytogenes, Helicobacter pylori, Mycobacterium avium, Mycobacterium smegmatis, Neisseria meningitidis, P. aeruginosa, Pertussis acellular, Propionibacterium acnes, Proteus mirabilis, Pseudomonas aeruginosa, S. aureus, S. pneumoniae, Serratia marcescens, Shigella flexneri, Shigella sonnei, Staphylococcus aureaus, Staphylococcus epidermidis, Streptococcus pneumoniae, Streptococcus pyogenes and Yersinia pestis.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

TABLE I

| Category | Hu-SCID Model | Virulence | Murine | Human |
|---|---|---|---|---|
| Histidine triad | Pneumococcal histidine protein A (PhpA, | C3 complement degradation activity (2) | b (25, 65) | e (2, 26) |
| proteins | BHV-11) | | | |
| Choline | Pneumococcal surface protein A | Interfere with complement deposition, Blocking recruitment of | b (66) | c (17), |
| binding | | ACP proteins, anti-bactericidal working by (apo)lactoferrin | | d (16, 67), |
| proteins | | binding (2) | | e (2, 18) |
| | Pneumococcal surface protein C | Interaction with complement component C3 and H, Interaction | b (68) | d (69) |
| | | with glycoconjugates and sialic acid and with pIgR (50) | | |
| Adhesins | Pneumococcal surface adhesin A | Adherence via E-cadherin (70), and N acetyl glycosamine and | b (71) | c (72, 73), |
| | | Metal ion transport (1) | | d (74), e (2) |
| | ORF SP_0082 | Fibronectin binding adhesion (32) | NA | d (33) |
| | Fructose biphosphate aldolase | Glycolysis, adhesion via flamingo cadherin receptor (36) | b (15) | c (15) |
| Degradation | Endo-β-N-acetylglucosaminidase | Degradation host polymers, facilitation colonization (38) | b (35) | d (39) |
| ECM | Zinc metalloprotease B | Proteolytic activity, zmpB mutant attenuated virulence (75) | a (33) | NA |
| | Zinc metalloprotease C | Proteolytic activation of human MMP9, zmpC mutant attenuated | NA | NA |
| | | virulence (14) | | |
| | IgA1 protease | Cleaving human IgA, facilitation of colonization (43) | NA | d (45) |
| | Serine protease PrtA | prtA mutant attenuated virulence (45) | b (35) | d (35) |
| | α-enolase | Glycolysis, plasminogen binding protein (46) | NA | d (49) |
| | Glyceraldehyde-3-phosphate | Glycolysis, plasminogen binding protein (46) | b (15) | c (15) |
| | dehydrogenase | | | |
| Transporters | ABC sugar transporter, sugar-binding | NA | NA | NA |
| | protein (SP_1683) | | | |
| | ABC transporter (Maltose/maltodextrin) | NA | NA | NA |
| | PTS system IIA component (mannose) | NA | NA | NA |
| | ABC transporters for glutamine (glnQ) | glnQ mutant attenuated virulence (12) | NA | NA |
| | ABC transporters for spermidine/putrescie | potD mutant attenuated virulence (12, 50) | b (51) | NA |
| | (potD) | | | |
| Stress proteins | DnaK | NA | a (53), b* | c, d, (15) |
| | GroEL | NA | (54) | NA |
| | ClpP protease | clp mutant attenuated virulence (57) | a (57, 58) | NA |
| Physiological | Pyruvate oxidase | H₂O₂ production (59, 60), spxB mutant attenuated virulence (61) | NA | c (15) |
| processes | Phosphoglycerate kinase | NA | NA | c (15) |
| | Pyruvate kinase | NA | NA | NA |
| Hypothetical | SP_1290, and SP_0562 | NA | NA | NA |
| proteins | | | | |

a Antigenic in mice models;
b Protective in mice models;
b* Not protective in mice models;
c Detected in sera obtained from children attending day care centra or/and healthy adults;
d Detected in sera obtained from infected individuals;
e proteins that are used in clinical trials;
NA not available.

1-38. (canceled)

39. A substantially pure or isolated antigen comprising the S. pneumoniae antigen SP_{—}1683, or a fragment thereof, for use in the treatment or prevention of an S. pneumoniae infection in a subject.

40. The use of S. pneumoniae antigen SP_{—}1683, or a fragment thereof, in combination with an antigen selected from the group consisting of the isolated, recombinant or synthetic S. pneumoniae human immunogenic antigens of SP_{—}0562, SP_{—}0965, SP_{—}0082, and SP_{—}1386, or fragments thereof, in the treatment or prevention of a S. pneumoniae infection in a subject in need thereof.

41. The substantially pure or isolated antigen of claim 39 in combination with at least one human immunogenic antigen of S. pneumoniae selected from the group consisting of Zinc metalloprotease B (zmpB), GroEL, and ABC transporter for spermidine/putrescie (potD), or a fragment thereof, or a substantially identical antigen for the treatment or prevention of a S. pneumoniae infection in a subject in need thereof.

42. The substantially pure or isolated antigen of claim 39 in combination with at least one human immunogenic antigen of the group consisting of Pneumococcal hisitidine antigen A (PhpA, BHV-11), Pneumococcal surface antigen A, Pneumococcal surface antigen C, Pneumococcal surface adhesin A, ORF SP_{—}0082, Frucose biphosphate aldolase, Endo-B-N-acetylglucosaminidase, IgAl protease, Serine protease PrtA, a-enolase, Glyceraldehyde-3-phosphate dehydrogenase, DnaK, Pyruvate oxidase, C1pP protease and Phosphoglycerate kinase, or a fragment thereof, or a substantially identical antigen for the treatment or prevention of a S. pneumoniae infection in a subject in need thereof.

43. The substantially pure or isolated antigen of claim 39 wherein the antigen is a peptide, protein, or polypeptide.

44. The substantially pure or isolated antigen of claim 39 wherein the antigen is a nucleotide.

45. The substantially pure or isolated antigen of claim 44 wherein the nucleotide is operably linked to a vector.

46. The substantially pure or isolated antigen of claim 39 in combination with an immunostimulant for use as a vaccination agent in a vaccination treatment of S. pneumoniae disorder in a human or against S. pneumoniae in a human.

47. An antibody that specifically binds to SP_{—}1683, or a fragment thereof, or a substantially identical antigen for the treatment of a S. pneumoniae infection in a subject in need thereof.

48. The use of substantially pure or isolated antibodies that specifically bind to SP_{—}1683, or a fragment thereof, for the treatment of a S. pneumoniae infection in a subject in need thereof.

49. The antibody of claim 47 in combination with one or more antibodies that specifically bind to at least one human immunogenic antigen of S. pneumoniae selected from the group consisting of Zinc metalloprotease B (zmpB), GroEL, and ABC transporter for spermidine/putrescie (potD), or a fragment thereof, or a substantially identical antigen for the treatment of a S. pneumoniae infection in a subject in need thereof.

50. The antibody of claim 47 in combination with one or more antibodies that specifically bind to at least one human immunogenic antigen of S. pneumoniae selected from the group consisting of Pneumococcal histidine antigen A (PhpA, BHV-11), Pneumococcal surface antigen A, Pneumococal surface antigen C, Pneumococcal surface adhesin A, ORF SP_{—}0082, Fructose bisphosphate aldolase, Endo-B-N-acetylglucosaminidase, IgAl protease, Serine protease PrtA, α-enolase, Glyceraldehyde-3-phosphate dehydrogenase, DnaK, Pyruvate oxidase, C1pP protease and Phosphoglycerate kinase, or a fragment thereof, or a substantially identical antigen for the treatment of a S. pneumoniae infection in a subject in need thereof.

51. The antibody of claim 47 wherein the antibody is a recombinant human antibody.

52. The antibody of claim 47 wherein the antibody is selected from the group consisting of a monoclonal antibody, a diclonal antibody, an oligoclonal antibody, a polyclonal antibody, a rearranged antibody or a heterohybrid antibody.

53. A substantially pure or isolated disease related antigen comprising a Periplasmic Binding Protein (PBP) selected from the group consisting of SP_{—}1683 and SP_{—}1386, or a fragment of said PBPs or substantially identical antigen for diagnosing a S. pneumoniae disorder in a human.

54. The substantially pure or isolated disease related antigen of claim 53 comprising SP_{—}1683, or a fragment thereof, or a substantially identical antigen for diagnosing a S. pneumoniae disorder in a human.

55. The substantially pure or isolated disease related antigen of claim 53 in combination with at least one human immunogenic antigen selected from the group consisting of Zinc metalloprotease B (zmpB), GroEL, and ABC transporter for spermidine/putrescie (potD), or a fragment thereof, or a substantially identical antigen for diagnosing a S. pneumoniae disorder in a human.

56. The substantially pure or isolated disease related antigen of claim 53 in combination with at least one human immunogenic antigen selected from the group consisting of Pneumococcal histidine antigen A (PhpA, BHV-11), Pneumococcal surface antigen A, Pneumococal surface antigen C, Pneumococcal surface adhesin A, ORF SP_{—}0082, Fructose bisphosphate aldolase, Endo-β-N-acetylglucosaminidase, IgA1 protease, Serine protease PrtA, α-enolase, Glyceraldehyde-3-phosphate dehydrogenase, DnaK, Pyruvate oxidase, C1pP protease and Phosphoglycerate kinase, or a fragment thereof, or a substantially identical antigen for diagnosing a S. pneumoniae disorder in a human.

57. The substantially pure or isolated disease related antigen of claim 53 wherein said antigen is a peptide, protein or polypeptide.

58. The substantially pure or isolated disease related antigen of claim 53 wherein said antigen is a nucleotide.
